(19) United States
(12) Patent Application Publication
(10) Pub. No.: US 2020/0338129 A1
(43) Pub. Date: Oct. 29, 2020

(54) COMPOSITIONS INCLUDING ENUCLEATED ERYTHROID CELLS

(71) Applicant: Rubius Therapeutics, Inc., Cambridge, MA (US)

(72) Inventors: Ho Ki Keith Wong, Newton, MA (US); Jie Li, Belmont, MA (US)

(21) Appl. No.: 16/857,712

(22) Filed: Apr. 24, 2020

Related U.S. Application Data
(60) Provisional application No. 62/839,506, filed on Apr. 26, 2019.

(51) Int. Cl. A61K 35/18; C12N 5/078; A61K 47/69

(52) U.S. Cl. CPC A61K 35/18; C12N 5/0641; A61K 47/6901; C12N 2500/16; C12N 2500/30; C12N 2500/42; C12N 2500/40; C12N 2500/34; C12N 2500/14

(57) ABSTRACT

Provided herein are compositions including enucleated erythroid cells and methods of making and using the same.

### CROSS-REFERENCE TO RELATED APPLICATIONS

This application claims priority to U.S. Provisional Patent Application Ser. No. 62/839,506, filed Apr. 26, 2019; the entire contents of which are herein incorporated by reference.

### TECHNICAL FIELD

The present invention relates generally to compositions containing enucleated erythroid cells.

### BACKGROUND

Red blood cells are transfused to patients who have experienced blood loss. In addition, engineered enucleated erythroid cells, including red blood cells, are in development as therapeutic agents which carry or present exogenous protein(s) to patients in need thereof.

### SUMMARY

The present invention is based on the discovery that compositions including (a) a population of enucleated erythroid cells and (b) a pharmaceutically acceptable aqueous buffered solution having a pH of about 6.5 to about 8.5 and an osmolarity of about 150 mOsm/L to about 400 mOsm/L including: about 5 mM to about 80 mM of a buffer, about 5 mM to about 35 mM phosphate ion, about 50 mM to about 160 mM sodium ion, about 5 mM to about 60 mM potassium ion, about 0.01 mM to about 10 mM calcium ion, about 1 mM to about 20 mM magnesium ion, and about 5 mM to about 60 mM of a non-ionic cell impermeant agent, and including less than 0.1 mM glucose, and optionally, not including one or more of sucrose, a colloid, and an antioxidant, have improved stability (e.g., a decrease in hemolysis and/or an increased cell count, after incubation at any of the exemplary temperatures described herein) as compared to other compositions including enucleated erythroid cells. In view of this discovery, provided herein are compositions that include (a) a population of enucleated erythroid cells; and (b) a pharmaceutically acceptable aqueous buffered solution having a pH of about 6.5 to about 8.5 and an osmolarity of about 150 mOsm/L to about 400 mOsm/L that includes: about 5 mM to about 80 mM of a buffer; about 5 mM to about 35 mM phosphate ion; about 50 mM to about 160 mM sodium ion; about 5 mM to about 60 mM potassium ion; about 0.01 mM to about 10 mM calcium ion; about 1 mM to about 20 mM magnesium ion; and about 5 mM to about 60 mM of a non-ionic cell impermeant agent, where: the pharmaceutically acceptable aqueous buffered solution includes less than 0.1 mM glucose; and optionally, the pharmaceutically acceptable aqueous buffered solution does not include one or more of: sucrose, a colloid, and an antioxidant. Also provided herein are kits that include any of these compositions, methods of making any of these compositions, and methods of treating a subject in need thereof that include administering any of these compositions.

In one aspect, provided here in are compositions that include: (a) a population of enucleated erythroid cells; and (b) a pharmaceutically acceptable aqueous buffered solution having a pH of 6.5 to 8.5 and an osmolarity of 150 mOsm/L to 400 mOsm/L comprising: about 5 mM to about 80 mM of a buffer; about 5 mM to about 35 mM phosphate ion; about 50 mM to about 160 mM sodium ion; about 5 mM to about 60 mM potassium ion; about 0.01 mM to about 10 mM calcium ion; about 1 mM to about 20 mM magnesium ion; and about 5 mM to about 60 mM of a non-ionic cell impermeant agent, where: the pharmaceutically acceptable aqueous buffered solution includes less than 5 mM glucose; and optionally, the pharmaceutically acceptable aqueous buffered solution does not include one or more of: sucrose, a colloid, and an antioxidant. In some embodiments, the pharmaceutically acceptable aqueous buffered solution includes about 10 mM to about 40 mM of the buffer. In some embodiments, the pharmaceutically acceptable aqueous buffered solution includes about 20 mM to about 30 mM of the buffer. In some embodiments, the buffer is a Good's buffer. In some embodiments, the Good's buffer is selected from the group consisting of: HEPES, MOPS, TES, MES, ADA, ACES, BES, Bicine, CAPS, CAPSO, CHES, PIPES, TAPS, and Tris. In some embodiments, the Good's buffer is HEPES. In some embodiments, the pharmaceutically acceptable aqueous buffered solution includes about 5 mM to about 25 mM phosphate ion. In some embodiments, the pharmaceutically acceptable aqueous buffered solution includes about 5 mM to about 15 mM phosphate ion. In some embodiments, the phosphate ion is present in the pharmaceutically acceptable aqueous buffered solution as monosodium phosphate and/or disodium phosphate. In some embodiments, the pharmaceutically acceptable aqueous buffered solution includes about 50 mM to about 140 mM sodium ion. In some embodiments, the pharmaceutically acceptable aqueous buffered solution includes about 70 mM to about 120 mM sodium ion. In some embodiments, the sodium ion is present in the pharmaceutically acceptable aqueous buffered solution as sodium chloride, monosodium phosphate, and/or disodium phosphate. In some embodiments, the pharmaceutically acceptable aqueous buffered solution includes about 10 mM to about 50 mM potassium ion. In some embodiments, the pharmaceutically acceptable aqueous buffered solution includes about 30 mM to about 50 mM potassium ion. In some embodiments, the potassium ion is present in the pharmaceutically acceptable aqueous buffered solution as potassium chloride. In some embodiments, the pharmaceutically acceptable aqueous buffered solution includes about 0.01 mM to about 5 mM calcium ion. In some embodiments, the pharmaceutically acceptable aqueous buffered solution includes about 0.01 mM to about 0.5 mM calcium ion. In some embodiments, the calcium ion is present in the pharmaceutically acceptable aqueous buffered solution as calcium chloride. In some embodiments, the pharmaceutically acceptable aqueous buffered solution includes about 1 mM to about 10 mM magnesium ion. In some embodiments, the pharmaceutically acceptable aqueous buffered solution includes about 3 mM to about 7 mM magnesium ion. In some embodiments, the magnesium ion is present in the pharmaceutically acceptable aqueous buffered solution as magnesium chloride. In some embodiments, the pharmaceutically acceptable aqueous buffered solution further includes about 20 mM to about 120 mM of an anionic cell impermeant agent. In some embodiments, the pharmaceutically acceptable aqueous buffered solution includes about 75 mM to about 120 mM of the anionic cell impermeant agent. In some embodiments, the pharmaceutically acceptable aqueous buffered solution includes about 90 mM to about 110 mM of the anionic cell impermeant agent. In some embodiments, the anionic cell impermeant agent is selected from the group of: lactobionate, citrate, and gluconate. In some embodiments, the anionic cell impermeant agent is lactobionate. In some embodiments, the pharmaceutically acceptable aqueous buffered solution includes about 20 mM to about 60 mM of the non-ionic cell impermeant agent. In some embodiments, the pharmaceutically acceptable aqueous buffered solution includes about 30 mM to about 50 mM of the non-ionic cell impermeant agent. In some embodiments, the non-ionic cell impermeant agent is selected from the group consisting of: mannitol, raffinose, and sucrose. In some embodiments, the non-ionic cell impermeant agent is mannitol. In some embodiments, the pharmaceutically acceptable aqueous buffered solution further includes about 1 mM to about 20 mM chloride ion. In some embodiments, the pharmaceutically acceptable aqueous buffered solution includes about 5 mM to about 15 mM chloride ion. In some embodiments, the pharmaceutically acceptable aqueous buffered solution further includes one or more of: about 0.01 mM to about 5 mM of a nucleobase, about 0.01 mM to about 5 mM of a nucleoside, and about 0.01 mM to about 5 mM of a nucleotide. In some embodiments, the pharmaceutically acceptable aqueous buffered solution further includes one or more of: about 0.01 mM to about 5 mM adenine, about 0.01 mM to about 5 mM adenosine, about 0.01 mM to about 5 mM adenosine monophosphate, about 0.01 mM to about 5 mM adenosine diphosphate, and about 0.01 mM to about 5 mM adenosine triphosphate. In some embodiments, the pharmaceutically acceptable aqueous buffered solution further includes about 3 mM to about 10 mM bicarbonate ion. In some embodiments, the pharmaceutically acceptable aqueous buffered solution further includes about 3 mM to about 7 mM bicarbonate ion. In some embodiments, the biocarbonate ion is present in the pharmaceutically acceptable aqueous buffered solution as sodium bicarbonate. In some embodiments, the pharmaceutically acceptable aqueous buffered solution further includes about 0.01 mM to about 5 mM pyruvate. In some embodiments, the pharmaceutically acceptable aqueous buffered solution further includes a poloxamer. In some embodiments, the poloxamer is poloxamer-188. In some embodiments, the pharmaceutically acceptable aqueous buffered solution includes about 0.01% w/v to about 2.0% w/v of the poloxamer. In some embodiments, the pharmaceutically acceptable aqueous buffered solution includes about 0.01% w/v to about 1.0% w/v of the poloxamer. In some embodiments, the pharmaceutically acceptable aqueous buffered solution includes about 0.3% w/v to about 0.7% w/v of the poloxamer. In some embodiments, the pharmaceutically acceptable aqueous buffered solution further includes human serum albumin. In some embodiments, the pharmaceutically acceptable aqueous buffered solution includes about 0.01% w/v to about 2.0% w/v human serum albumin. In some embodiments, the pharmaceutically acceptable aqueous buffered solution includes about 0.1% w/v to about 0.3% w/v human serum albumin. In some embodiments, the pharmaceutically acceptable aqueous buffered solution has a pH of about 7.0 to about 8.0. In some embodiments, the pharmaceutically acceptable aqueous buffered solution has a pH of about 7.2 to about 7.6. In some embodiments, the pharmaceutically acceptable aqueous buffered solution has an osmolarity of about 250 mOsm/L to about 400 mOsm/L. In some embodiments, the pharmaceutically acceptable aqueous buffered solution has an osmolarity of about 300 mOsm/L to about 400 mOsm/L. In some embodiments, the pharmaceutically acceptable aqueous buffered solution includes less than 0.01 mM glucose. In some embodiments, the pharmaceutically acceptable aqueous buffered solution includes less than about 0.001 mM glucose. In some embodiments, the pharmaceutically acceptable aqueous buffered solution includes no glucose. In some embodiments, the composition includes about 1.0×10⁹ to about 7.0×10⁹ enucleated erythroid cells/mL. In some embodiments, the composition includes about 2.0×10⁹ to about 4.0×10⁹ enucleated erythroid cells/mL. In some embodiments, the composition includes about 4.0×10⁹ to about 6.0×10⁹ enucleated erythroid cells/mL. In some embodiments, the enucleated erythroid cells are human enucleated erythroid cells. In some embodiments, the enucleated erythroid cells are donor human enucleated erythroid cells. In some embodiments, the enucleated erythroid cells are engineered human enucleated erythroid cells. In some embodiments, the engineered human enucleated erythroid cells include one or more exogenous protein(s). In some embodiments, the engineered human enucleated erythroid cells are click-conjugated human enucleated erythroid cells. In some embodiments, the engineered human enucleated erythroid cells have been hypotonically loaded. In some embodiments, the engineered human enucleated erythroid cells have been loaded by physical manipulation. In some embodiments, one of the one or more exogenous protein(s) is present in the cytosol of the engineered human enucleated erythroid cells. In some embodiments, one of the one or more exogenous protein is a protein present on the membrane of the engineered human enucleated erythroid cells. In some embodiments, one of the one or more exogenous protein(s) is phenylalanine ammonia lyase, wherein the phenylalanine ammonia lyase (PAL) is present in the cytosol of the engineered human enucleated erythroid cell. In some embodiments, storage of the composition at about 2° C. to about 10° C. for 30 days to about 100 days results in less than 10% hemolysis. In some embodiments, storage of the composition at about 2° C. to about 10° C. for 30 days to about 100 days results in less than 8% hemolysis. In some embodiments, storage of the composition at about 2° C. to about 10° C. for 30 days to about 100 days results in less than a 10% decrease in cell density. In some embodiments, storage of the composition at about 2° C. to about 10° C. for 30 days to about 100 days results in less than 8% hemolysis. In some embodiments, the pharmaceutically acceptable aqueous buffered solution does not include an antioxidant agent. In some embodiments, the pharmaceutically acceptable aqueous buffered solution does not include a colloid. In some embodiments, the colloid is a dextran. In some embodiments, the pharmaceutically acceptable aqueous buffered solution does not include an antioxidant agent and does not include a colloid.

In another aspect, provided is a method of treating a subject, the method includes: (i) providing a composition of any of the above embodiments, that has been stored at a temperature of about 2° C. to about 10° C. for a period of time; and (ii) administering the composition of step (i) to a subject in need thereof.

In some embodiments, provided is a method of treating a subject having phenylketonuria, then method includes (i) providing a composition of where the one or more exogenous protein(s) is phenylalanine ammonia lyase (PAL), where the phenylalanine ammonia lyase is present in the cytosol of the engineered human enucleated erythroid cell that has been stored at a temperature of about 2° C. to about 10° C. for a period of time; and (ii) administering the composition of step (i) to the subject in need thereof. In some embodiments, the method further includes between step (i) and step (ii) a step of warming the composition of step (i) to a temperature of about 15° C. to about 30° C. In some embodiments, the composition has been stored at a temperature of about 4° C. to about 6° C. In some embodiments, the period of time is about 30 days to about 100 days. In some embodiments, the period of time is about 35 days to about 60 days. In some embodiments, the period of time is about 45 days to about 60 days. In some embodiments, the composition is warmed to a temperature of about 20° C. to about 30° C. In some embodiments, the composition is warmed to a temperature of about 23° C. to about 27° C. In some embodiments, less than 10% hemolysis occurs following step (i) as compared to the composition prior to storage at a temperature of about 2° C. to about 10° C. for the period of time. In some embodiments, less than 8% hemolysis occurs following step (i) as compared to the composition prior to storage at a temperature of about 2° C. to about 10° C. for the period of time. In some embodiments, less than a 10% decrease in cell density occurs following step (i) as compared to the composition prior to storage at a temperature of about 2° C. to about 10° C. for the period of time. In some embodiments, less than a 8% decrease in cell density occurs following step (i) as compared to the composition prior to storage at a temperature of about 2° C. to about 10° C. for the period of time. In some embodiments, step (ii) includes intravenous administration to the subject.

In another aspect, provided is a method of treating a subject, the method includes administering the composition of any of the above embodiments to a subject in need thereof. In some embodiments, the composition was previously stored at a temperature of about 2° C. to about 10° C. for a period of time. In some embodiments, the composition was previously stored at a temperature of about 4° C. to about 6° C. In some embodiments, the period of time is about 30 days to about 100 days. In some embodiments, the period of time is about 35 days to about 60 days. In some embodiments, the period of time is about 45 days to about 60 days. In some embodiments, the method further includes prior to the administering step, a step of warming the composition to a temperature of about 15° C. to about 30° C. In some embodiments, the composition is warmed to a temperature of about 20° C. to about 30° C. In some embodiments, the composition is warmed to a temperature of about 23° C. to about 27° C. In some embodiments, prior to the administering, the composition has been stored at a temperature of about 2° C. to about 10° C. for a period of time, and less than 10% hemolysis occurs following storage at a temperature of about 2° C. to about 10° C. for the period of time as compared to the composition prior to storage at a temperature of about 2° C. to about 10° C. for the period of time. In some embodiments, less than 8% hemolysis occurs following storage at a temperature of about 2° C. to about 10° C. for the period of time as compared to the composition prior to storage at a temperature of about 2° C. to about 10° C. for the period of time. In some embodiments, prior to the administering, the composition has been stored at a temperature of about 2° C. to about 10° C. for a period of time, and wherein less than a 10% decrease in cell density occurs following storage at a temperature of about 2° C. to about 10° C. for the period of time as compared to the composition prior to storage at a temperature of about 2° C. to about 10° C. for the period of time. In some embodiments, less than a 8% decrease in cell density occurs following storage at a temperature of about 2° C. to about 10° C. for the period of time as compared to the composition prior to storage at a temperature of about 2° C. to about 10° C. for the period of time. In some embodiments, the administering step includes intravenous administration to the subject.

In another aspect, provided is a method of making a composition, the method includes: (i) providing a population of enucleated erythroid cells; and (ii) resuspending the population of enucleated erythroid cells in a pharmaceutically acceptable aqueous buffered solution having a pH of 6.5 to 8.5 and an osmolarity of 150 mOsm/L to 400 mOsm/L that includes: about 5 mM to about 80 mM of a buffer; about 5 mM to about 35 mM phosphate ion; about 50 mM to about 160 mM sodium ion; about 5 mM to about 60 mM potassium ion; about 0.01 mM to about 10 mM calcium ion; about 1 mM to about 20 mM magnesium ion; and about 5 mM to about 60 mM of a non-ionic cell impermeant agent, wherein: the pharmaceutically acceptable aqueous buffered solution includes less than 5 mM glucose; and optionally, the pharmaceutically acceptable aqueous buffered solution does not include one or more of: sucrose, a colloid, and an antioxidant. In some embodiments, the pharmaceutically acceptable aqueous buffered solution includes about 10 mM to about 40 mM of the buffer. In some embodiments, the pharmaceutically acceptable aqueous buffered solution includes about 20 mM to about 30 mM of the buffer. In some embodiments, the buffer is a Good's buffer. In some embodiments, the Good's buffer is selected from the group consisting of: HEPES, MOPS, TES, MES, ADA, ACES, BES, Bicine, CAPS, CAPSO, CHES, PIPES, TAPS, and Tris. In some embodiments, the Good's buffer is HEPES. In some embodiments, the pharmaceutically acceptable aqueous buffered solution includes about 5 mM to about 25 mM phosphate ion. In some embodiments, the pharmaceutically acceptable aqueous buffered solution includes about 5 mM to about 15 mM phosphate ion. In some embodiments, the phosphate ion is present in the pharmaceutically acceptable aqueous buffered solution as monosodium phosphate and/or disodium phosphate. In some embodiments, the pharmaceutically acceptable aqueous buffered solution includes about 50 mM to about 140 mM sodium ion. In some embodiments, the pharmaceutically acceptable aqueous buffered solution includes about 70 mM to about 120 mM sodium ion. In some embodiments, the sodium ion is present in the pharmaceutically acceptable aqueous buffered solution as sodium chloride, monosodium phosphate, and/or disodium phosphate. In some embodiments, the pharmaceutically acceptable aqueous buffered solution includes about 10 mM to about 50 mM potassium ion. In some embodiments, the pharmaceutically acceptable aqueous buffered solution includes about 30 mM to about 50 mM potassium ion. In some embodiments, the potassium ion is present in the pharmaceutically acceptable aqueous buffered solution as potassium chloride. In some embodiments, the pharmaceutically acceptable aqueous buffered solution includes about 0.01 mM to about 5 mM calcium ion. In some embodiments, the pharmaceutically acceptable aqueous buffered solution includes about 0.01 mM to about 0.5 mM calcium ion. In some embodiments, the calcium ion is present in the pharmaceutically acceptable aqueous buffered solution as calcium chloride. In some embodiments, the pharmaceutically acceptable aqueous buffered solution includes about 1 mM to about 10 mM magnesium ion. In some embodiments, the pharmaceutically acceptable aqueous buffered solution includes about 3 mM to about 7 mM magnesium ion. In some embodiments, the magnesium ion is present in the pharmaceutically acceptable aqueous buffered solution as magnesium chloride. In some embodiments, the pharmaceutically acceptable aqueous buffered solution further includes about 20 mM to about 120 mM of an anionic cell impermeant agent. In some embodiments, the pharmaceutically acceptable aqueous buffered solution includes about 75 mM to about 120 mM of the anionic cell impermeant agent. In some embodiments, the pharmaceutically acceptable aqueous buffered solution includes about 90 mM to about 110 mM of the anionic cell impermeant agent. In some embodiments, the anionic cell impermeant agent is selected from the group of: lactobionate, citrate, and gluconate. In some embodiments, the anionic cell impermeant agent is lactobionate. In some embodiments, the pharmaceutically acceptable aqueous buffered solution includes about 20 mM to about 60 mM of the non-ionic cell impermeant agent. In some embodiments, the pharmaceutically acceptable aqueous buffered solution includes about 30 mM to about 50 mM of the non-ionic cell impermeant agent. In some embodiments, the non-ionic cell impermeant agent is selected from the group consisting of: mannitol, raffinose, and sucrose. In some embodiments, the non-ionic cell impermeant agent is mannitol. In some embodiments, the pharmaceutically acceptable aqueous buffered solution further includes about 1 mM to about 20 mM chloride ion. In some embodiments, the pharmaceutically acceptable aqueous buffered solution includes about 5 mM to about 15 mM chloride ion. In some embodiments, the pharmaceutically acceptable aqueous buffered solution further includes one or more of: about 0.01 mM to about 5 mM of a nucleobase, about 0.01 mM to about 5 mM of a nucleoside, and about 0.01 mM to about 5 mM of a nucleotide. In some embodiments, the pharmaceutically acceptable aqueous buffered solution further includes one or more of: about 0.01 mM to about 5 mM adenine, about 0.01 mM to about 5 mM adenosine, about 0.01 mM to about 5 mM adenosine monophosphate, about 0.01 mM to about 5 mM adenosine diphosphate, and about 0.01 mM to about 5 mM adenosine triphosphate. In some embodiments, the pharmaceutically acceptable aqueous buffered solution further includes about 3 mM to about 10 mM bicarbonate ion. In some embodiments, the pharmaceutically acceptable aqueous buffered solution further includes about 3 mM to about 7 mM bicarbonate ion. In some embodiments, the biocarbonate ion is present in the pharmaceutically acceptable aqueous buffered solution as sodium bicarbonate. In some embodiments, the pharmaceutically acceptable aqueous buffered solution further includes about 0.01 mM to about 5 mM pyruvate. In some embodiments, the pharmaceutically acceptable aqueous buffered solution further includes a poloxamer. In some embodiments, the poloxamer is poloxamer-188. In some embodiments, the pharmaceutically acceptable aqueous buffered solution includes about 0.01% w/v to about 2.0% w/v of the poloxamer. In some embodiments, the pharmaceutically acceptable aqueous buffered solution includes about 0.01% w/v to about 1.0% w/v of the poloxamer. In some embodiments, the pharmaceutically acceptable aqueous buffered solution includes about 0.3% w/v to about 0.7% w/v of the poloxamer. In some embodiments, the pharmaceutically acceptable aqueous buffered solution further includes human serum albumin. In some embodiments, the pharmaceutically acceptable aqueous buffered solution includes about 0.01% w/v to about 2.0% w/v human serum albumin. In some embodiments, the pharmaceutically acceptable aqueous buffered solution includes about 0.1% w/v to about 0.3% w/v human serum albumin. In some embodiments, the pharmaceutically acceptable aqueous buffered solution has a pH of about 7.0 to about 8.0. In some embodiments, the pharmaceutically acceptable aqueous buffered solution has a pH of about 7.2 to about 7.6. In some embodiments, the pharmaceutically acceptable aqueous buffered solution has an osmolarity of about 250 mOsm/L to about 400 mOsm/L. In some embodiments, the pharmaceutically acceptable aqueous buffered solution has an osmolarity of about 300 mOsm/L to about 400 mOsm/L. In some embodiments, the pharmaceutically acceptable aqueous buffered solution includes less than 0.01 mM glucose. In some embodiments, the pharmaceutically acceptable aqueous buffered solution includes less than about 0.001 mM glucose. In some embodiments, the pharmaceutically acceptable aqueous buffered solution includes no glucose. In some embodiments, the composition includes about 1.0×10⁹ to about 7.0×10⁹ enucleated erythroid cells/mL. In some embodiments, the composition includes about 2.0×10⁹ to about 4.0×10⁹ enucleated erythroid cells/mL. In some embodiments, the composition includes about 4.0×10⁹ to about 6.0×10⁹ enucleated erythroid cells/mL. In some embodiments, the enucleated erythroid cells are human enucleated erythroid cells. In some embodiments, the enucleated erythroid cells are donor human enucleated erythroid cells. In some embodiments, the enucleated erythroid cells are engineered human enucleated erythroid cells. In some embodiments, the engineered human enucleated erythroid cells include one or more exogenous protein(s). In some embodiments, the engineered human enucleated erythroid cells are click-conjugated human enucleated erythroid cells. In some embodiments, the engineered human enucleated erythroid cells have been hypotonically loaded. In some embodiments, the engineered human enucleated erythroid cells have been loaded by physical manipulation. In some embodiments, one of the one or more exogenous protein(s) is present in the cytosol of the engineered human enucleated erythroid cells. In some embodiments, one of the one or more exogenous protein is a protein present on the membrane of the engineered human enucleated erythroid cells. In some embodiments, one of the one or more exogenous protein(s) is phenylalanine ammonia lyase, wherein the phenylalanine ammonia lyase is present in the cytosol of the engineered human enucleated erythroid cell. In some embodiments, storage of the composition at about 2° C. to about 10° C. for 30 days to about 100 days results in less than 10% hemolysis. In some embodiments, storage of the composition at about 2° C. to about 10° C. for 30 days to about 100 days results in less than 8% hemolysis. In some embodiments, storage of the composition at about 2° C. to about 10° C. for 30 days to about 100 days results in less than a 8% decrease in cell density. In some embodiments, the pharmaceutically acceptable aqueous buffered solution does not include an antioxidant agent. In some embodiments, the pharmaceutically acceptable aqueous buffered solution does not include a colloid. In some embodiments, the colloid is a dextran. In some embodiments, the pharmaceutically acceptable aqueous buffered solution does not include an antioxidant agent and does not include a colloid. In some embodiments, the method further includes culturing erythroid progenitor cells to provide the population of enucleated erythroid cells. In another aspect, provided is a composition provided by the methods described in any of the above embodiments.

The term “non-ionic cell impermeant agent” means a molecule that (i) does not have any cations and does not have any anions at a physiological pH (e.g., a pH of about 7.4), (ii) does not substantially cross the plasma membrane of an intact and a physically- and chemically-unaltered mammalian cell, and (iii) prevents water movement into an intact and a physically- and chemically-unaltered mammalian cell by passive biophysical osmotic effects. Non-limiting examples of non-ionic cell impermeant agents include mannitol, raffinose, sucrose, sorbitol, trehalose, gluconate, and a polyethylene glycol (PEG) (e.g., a PEG having a molecular weight of greater than 1 kDa, greater than 5 kDa, greater than 10 kDa, greater than 15 kDa, e.g., PEG 20 kDa). Additional examples of non-ionic cell impermeant agents are known in the art.

The term “anionic cell impermeant agent” means a molecule that (i) has one or more anion(s) at a physiological pH (e.g., a pH of about 7.4), (ii) does not substantially cross the plasma membrane of an intact and a physically- and chemically-unaltered mammalian cell, and (iii) prevents water movement into an intact and a physically- and chemically-unaltered mammalian cell by passive biophysical osmotic effects. Non-limiting examples of anionic cell impermeant agents include lactobionate, citrate, and gluconate. Additional examples of anionic cell impermeant agents are known in the art.

The term “population” means two or more of a given article (e.g., any of the exemplary enucleated erythroid cells described herein).

The term “engineered enucleated erythroid cell” means an enucleated erythroid cell (e.g., a human enucleated erythroid cell) that comprises one or more (e.g., two, three, four, five, or six) exogenous protein(s) (e.g., any combination of the exemplary exogenous proteins described herein or known in the art). For example, an engineered enucleated erythroid cell can have one or more exogenous protein(s) present in its cytosol. In some examples, an engineered enucleated erythroid cell can have one or more exogenous protein(s) present on its plasma membrane. In some examples, an engineered enucleated erythroid cell can have (i) one or more exogenous protein(s) present in its cytosol and (ii) one or more exogenous proteins present on its plasma membrane. Non-limiting examples of engineered enucleated erythroid cells include click-conjugated enucleated erythroid cells, enucleated erythroid cell that have been hypotonically loaded, and enucleated erythroid cells that have been loaded by physical manipulation (e.g., any of the exemplary types of physical manipulation described herein or known in the art). Additional non-limiting aspects of engineered enucleated erythroid cells are described herein.

The term “click-conjugated enucleated erythroid cell” means an engineered enucleated erythroid cell that has at least one exogenous protein conjugated to another protein (e.g., an endogenous protein of an enucleated red blood cell or different exogenous protein) present on the plasma membrane of an engineered enucleated erythroid cells through the catalytic activity of an enzyme(s) and/or peptide sequence(s), and/or a chemical reaction.

The term “hypotonically-loaded enucleated erythroid cell” means an engineered enucleated erythroid cell that was generated, at least in part, by exposing an enucleated erythroid cell or an erythroid progenitor cell to a low ionic strength buffer (e.g., any of the exemplary low ionic strength buffers described herein) comprising one or more exogenous protein(s). Non-limiting examples of methods that can be used to generate a hypotonically-loaded enucleated erythroid cell are described herein. Additional methods for generating a hypotonically-loaded enucleated erythroid cell are known in the art.

The term “enucleated erythroid cell loaded by physical manipulation” means an enucleated erythroid cell that was generated, at least in part, by physically manipulating an erythroid progenitor cell in a manner that results in the introduction of a nucleic acid encoding one or more exogenous protein(s) (e.g., any of the exemplary exogenous proteins described herein or known in the art) into the erythroid progenitor cell. Non-limiting examples of physical manipulation that can be used to introduce a nucleic acid encoding one or more exogenous protein(s) into an erythroid progenitor cell include electroporation and particle-mediated transfection. Additional examples of physical manipulation that can be used to introduce a nucleic acid encoding one or more exogenous protein(s) into an erythroid progenitor are known in the art.

The term “exogenous protein” refers to a protein that is introduced into or onto a cell, or is caused to be expressed by the cell by introducing an exogenous nucleic acid encoding the protein into the cell or into a progenitor of the cell. In some embodiments, an exogenous protein is a protein encoded by an exogenous nucleic acid that was introduced into the cell or a progenitor of the cell, which nucleic acid is optionally not retained by the cell. In some embodiments, an exogenous protein is a protein conjugated to the surface of the cell by chemical or enzymatic means. Non-limiting classes of exogenous proteins include enzymes, interleukins, cytokine receptors, Fc-binding molecules, T-cell activating ligands, T-cell receptors, immune inhibitory molecules, WIC molecules, APC-binding molecules, autoantigens, allergens, toxins, targeting agents, receptor ligands (e.g., receptor agonists or receptor antagonists), and antibodies or antibody fragments. Additional examples of exogenous proteins that can be present in an engineered enucleated erythroid cell are described herein (see, e.g., Tables A-D). Additional examples of exogenous proteins that can be present in engineered enucleated erythroid cells are known in the art.

The term “protein present on the membrane” means a (1) a protein that is physically attached to or at least partially embedded in the membrane of an enucleated erythroid cell (e.g., a transmembrane protein, a peripheral membrane protein, a lipid-anchored protein (e.g., a GPI-anchor, an N-myristolyated protein, or a S-palmitoylated protein)) or (2) a protein that is stably bound to its cognate receptor, where the cognate receptor is physically attached to the membrane of an enucleated erythroid cell (e.g., a ligand bound to its cognate receptor, where the cognate receptor is physically attached to the membrane of the enucleated erythroid cell). Non-limiting methods for determining the presence of protein on the membrane of a mammalian cell include fluorescence-activated cell sorting (FACS), immunohistochemistry, cell-fractionation assays and Western blotting.

The term “erythroid progenitor cells” means a mammalian cell that is capable of eventually differentiating/developing into an enucleated erythroid cell. In some embodiments, the erythroid progenitor cell is a cord blood stem cell, a CD34⁺ cell, a hematopoietic stem/progenitor cell (HSC, HSPC), a spleen colony forming (CFU-S) cell, a common myeloid progenitor (CMP) cell, a blastocyte colony-forming cell, a burst forming unit-erythroid/erythrocyte (BFU-E), a megakaryocyte-erythroid progenitor (MEP) cell, an erythroid colony-forming unit, or colony-forming unit erythrocyte (CFU-E), an induced pluripotent stem cell (iPSC), a mesenchymal stem cell (MSC), or a combination thereof.

The term “antioxidant agent” means agents that prevent chemical changes caused by exposure to oxygen and/or radical oxygen species, and includes both enzymatic and non-enzymatic agents. Non-limiting examples of enzymatic antioxidants include superoxide dismutase, glutathione peroxidase, and catalase. Non-limiting examples of nonenzymatic antioxidants include ascorbic acid (vitamin C), α-tocopherol (vitamin E), glutathione, N-acetyl cysteine, and (3-carotene (carotenoids). Additional examples of antioxidant agents are known in the art.

The term “subject” refers to any mammal. In some embodiments, the subject or “subject in need of treatment” can be a primate (e.g., a human, a simian (e.g., a monkey (e.g., marmoset or baboon), or an ape (e.g., a gorilla, chimpanzee, orangutan, or gibbon)), a rodent (e.g., a mouse, a guinea pig, a hamster, or a rat), a rabbit, a dog, a cat, a horse, a sheep, a cow, a pig, or a goat. In some embodiments, the subject or “subject suitable for treatment” may be a non-human mammal, especially mammals that are conventionally used as models for demonstrating therapeutic efficacy in humans (e.g., a mouse, a pig, a rat, or a non-human primate) may be employed. In some examples, a subject can be previously diagnosed or identified as being in need of treatment by a medical professional (e.g., a physician, a laboratory technician, a physician's assistant, a nurse, or a clinical laboratory technician).

As used herein, “treating” means a reduction in the number, severity, frequency, and/or duration of one or more symptoms of a medical disease or condition in a subject (e.g., any of the exemplary subjects described herein).

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A and 1B show the percent hemolysis and the percent change in cell count for enucleated erythroid cells stored in T1 series of formulations as compared to enucleated erythroid cells stored in HypoThermosol® (HTS; Sigma-Aldrich Cat. No. H4416). FIG. 1A shows the percent hemolysis for enucleated erythroid cells stored in T1 series of formulations as compared to those stored in HTS after 34, 40, and 68 days, respectively. FIG. 1B shows the percent change in cell count for enucleated erythroid cells stored in T1 series of formulations for 68 days as compared to those stored in HTS for 68 days.

FIG. 2 shows enucleated red blood cell concentration after storage in HTS or T1-1 for 32 days or 45 days, respectively.

FIGS. 3A and 3B show osmoscan curves of enucleated erythroidcells stored in the T1-1 formulation as compared to those stored in HTS. FIG. 3A shows the osmoscan curves of enucleated erythroid cells stored in the T1-1 formulation or in HTS after 34 days, 40 days, and 68 days, respectively. FIG. 3B shows the osmoscan curves of enucleated erythroid cells stored in the T1-1 formulation or in HTS after 32 days or 45 days, respectively.

FIG. 4A shows the cell concentration over time of engineered enucleated erythroid cells comprising a first exogenous protein comprising 4-1BBL and a second exogenous protein comprising IL-15 linked to an extracellular portion of IL-15 receptor alpha (IL-15Rα) on their cell surface, when stored at 2-8° C. in T1-1 (6 batches) or T1-1 further supplemented with 0.2% w/v human serum albumin (2 batches).

FIG. 4B shows the percentage of hemolysis over time of engineered enucleated erythroid cells comprising a first exogenous protein comprising 4-1BBL and a second exogenous protein comprising IL-15 linked to an extracellular portion of IL-15Rα on their surface, when stored at 2-8° C. in T1-1 (6 batches) or T1-1 further supplemented with 0.2% w/v human serum albumin (2 batches).

### DETAILED DESCRIPTION

Provided herein are compositions that include (a) a population of enucleated erythroid cells; and (b) a pharmaceutically acceptable aqueous buffered solution having a pH of about 6.5 to about 8.5 (e.g., any of the subranges of this range described herein) and an osmolarity of about 150 mOsm/L to about 400 mOsm/L (e.g., any of the subranges of this range described herein) including: about 5 mM to about 80 mM (e.g., any of the subranges of this range described herein) of a buffer (e.g., any of the exemplary buffers described herein or known in the art); about 5 mM to about 35 mM (e.g., any of the subranges of this range described herein) phosphate ion; about 50 mM to about 160 mM (e.g., any of the subranges of this range described herein) sodium ion; about 5 mM to about 60 mM potassium ion; about 0.01 mM to about 10 mM calcium ion; about 1 mM to about 20 mM (e.g., any of the subranges of this range described herein) magnesium ion; and about 5 mM to about 60 mM (e.g., any of the subranges of this range described herein) of a non-ionic cell impermeant agent (e.g., any of the exemplary non-ionic cell impermeant agents described herein or known in the art), where: the pharmaceutically acceptable aqueous buffered solution comprises less than 0.1 mM glucose (e.g., contains no detectable glucose); and optionally, the pharmaceutically acceptable aqueous buffered solution does not comprise one or more of: sucrose, a colloid, and an antioxidant. Some embodiments of these compositions include less than 0.005 mM glucose, less than 0.001 mM glucose, no glucose, or no detectable glucose. Some embodiments of these compositions do not include one or more (e.g., one, two, three, or four) of sucrose, a colloid (e.g., a dextran), and an antioxidant.

In some embodiments, storage of any of the compositions described herein at about 2° C. to about 10° C. (e.g., about 2° C. to about 9° C., about 2° C. to about 8° C., about 2° C. to about 7° C., about 2° C. to about 6° C., about 2° C. to about 5° C., about 2° C. to about 4° C., about 3° C. to about 10° C., about 3° C. to about 9° C., about 3° C. to about 8° C., about 3° C. to about 7° C., about 3° C. to about 6° C., about 3° C. to about 5° C., about 4° C. to about 10° C., about 4° C. to about 9° C., about 4° C. to about 8° C., about 4° C. to about 7° C., about 4° to about 6° C., about 5° C. to about 10° C., about 5° C. to about 9° C., about 5° C. to about 8° C., about 5° C. to about 7° C., about 6° C. to about 10° C., about 6° C. to about 9° C., about 6° C. to about 8° C., about 7° C. to about 10° C., about 7° C. to about 9° C., or about 8° C. to about 10° C.) for 30 days to about 100 days (e.g., about 30 days to about 95 days, about 30 days to about 90 days, about 30 days to about 85 days, about 30 days to about 80 days, about 30 days to about 75 days, about 30 days to about 70 days, about 30 days to about 65 days, about 30 days to about 60 days, about 30 days to about 55 days, about 30 days to about 50 days, about 30 days to about 45 days, about 30 days to about 40 days, about 30 days to about 35 days, about 35 days to about 100 days, about 35 days to about 95 days, about 35 days to about 90 days, about 35 days to about 85 days, about 35 days to about 80 days, about 35 days to about 75 days, about 35 days to about 70 days, about 35 days about to 65 days, about 35 days to about 60 days, about 35 days to about 55 days, about 35 days to about 50 days, about 35 days to about 45 days, about 35 days to about 40 days, about 40 days to about 100 days, about 40 days to about 95 days, about 40 days to about 90 days, about 40 days to about 85 days, about 40 days to about 80 days, about 40 days to about 75 days, about 40 days to about 70 days, about 40 days about to 65 days, about 40 days to about 60 days, about 40 days to about 55 days, about 40 days to about 50 days, about 40 days to about 45 days, about 45 days to about 100 days, about 45 days to about 95 days, about 45 days to about 90 days, about 45 days to about 85 days, about 45 days to about 80 days, about 45 days to about 75 days, about 45 days to about 70 days, about 45 days about to 65 days, about 45 days to about 60 days, about 45 days to about 55 days, about 45 days to about 50 days, about 50 days to about 100 days, about 50 days to about 95 days, about 50 days to about 90 days, about 50 days to about 85 days, about 50 days to about 80 days, about 50 days to about 75 days, about 50 days to about 70 days, about 50 days about to 65 days, about 50 days to about 60 days, about 50 days to about 55 days, about 55 days to about 100 days, about 55 days to about 95 days, about 55 days to about 90 days, about 55 days to about 85 days, about 55 days to about 80 days, about 55 days to about 75 days, about 55 days to about 70 days, about 55 days about to 65 days, about 55 days to about 60 days, about 60 days to about 100 days, about 60 days to about 95 days, about 60 days to about 90 days, about 60 days to about 85 days, about 60 days to about 80 days, about 60 days to about 75 days, about 60 days to about 70 days, about 60 days about to 65 days, about 65 days to about 100 days, about 65 days to about 95 days, about 65 days to about 90 days, about 65 days to about 85 days, about 65 days to about 80 days, about 65 days to about 75 days, about 65 days to about 70 days, about 70 days to about 100 days, about 70 days to about 95 days, about 70 days to about 90 days, about 70 days to about 85 days, about 70 days to about 80 days, about 70 days to about 75 days, about 75 days to about 100 days, about 75 days to about 95 days, about 75 days to about 90 days, about 75 days to about 85 days, about 75 days to about 80 days, about 80 days to about 100 days, about 80 days to about 95 days, about 80 days to about 90 days, about 80 days to about 85 days, about 85 days to about 100 days, about 85 days to about 95 days, about 85 days to about 90 days, about 90 days to about 100 days, about 90 days to about 95 days, about 95 days to about 100 days) results in less than 12% hemolysis, less than 10.0% hemolysis, less than 9.5% hemolysis, less than 9.0% hemolysis, less than 8.5% hemolysis, less than 8.0% hemolysis, less than 7.5% hemolysis, less than 7.0% hemolysis, less than 6.5% hemolysis, less than 6.0% hemolysis, less than 5.5% hemolysis, less than 5.0% hemolysis, less than 4.5% hemolysis, less than 4.0% hemolysis, less than 3.5% hemolysis, less than 3.0% hemolysis, less than 2.5% hemolysis, less than 2.0% hemolysis, less than 1.5% hemolysis, less than 1.0% hemolysis, less than 0.5% hemolysis, or less than 0.1% hemolysis (e.g., as compared to prior to storage).

Also provided herein are methods of treating a subject (e.g., any of the subjects described herein) that include (i) providing any composition described herein that has been stored at a temperature of about 2° C. to about 10° C. (e.g., any of the subranges of this range described herein) for a period of time (e.g., any of the exemplary periods of time described herein, e.g., about 30 days to about 100 days, or any of the subranges of this range described herein); and (ii) administering the composition of step (i) to a subject in need thereof. In some embodiments of these methods, less than 12% hemolysis (e.g., less than 10% hemolysis, less than 9.5% hemolysis, less than 9.0% hemolysis, less than 8.5% hemolysis, less than 8.0% hemolysis, less than 7.5% hemolysis, less than 7.0% hemolysis, less than 6.5% hemolysis, less than 6.0% hemolysis, less than 5.5% hemolysis, less than 5.0% hemolysis, less than 4.5% hemolysis, less than 4.0% hemolysis, less than 3.5% hemolysis, less than 3.0% hemolysis, less than 2.5% hemolysis, less than 2.0% hemolysis, less than 1.5% hemolysis, less than 1.0% hemolysis, less than 0.5% hemolysis, or less than 0.1% hemolysis) occurs following step (i) as compared to the composition prior to storage at a temperature of about 2° C. to about 10° C. (e.g., about 2° C. to about 9° C., about 2° C. to about 8° C., about 2° C. to about 7° C., about 2° C. to about 6° C., about 2° C. to about 5° C., about 2° C. to about 4° C., about 3° C. to about 10° C., about 3° C. to about 9° C., about 3° C. to about 8° C., about 3° C. to about 7° C., about 3° C. to about 6° C., about 3° C. to about 5° C., about 4° C. to about 10° C., about 4° C. to about 9° C., about 4° C. to about 8° C., about 4° C. to about 7° C., about 4° to about 6° C., about 5° C. to about 10° C., about 5° C. to about 9° C., about 5° C. to about 8° C., about 5° C. to about 7° C., about 6° C. to about 10° C., about 6° C. to about 9° C., about 6° C. to about 8° C., about 7° C. to about 10° C., about 7° C. to about 9° C., or about 8° C. to about 10° C.) for the period of time (e.g., any of the exemplary periods of time described herein, e.g., about 30 days to about 100 days, or any of the subranges of this range described herein). In some embodiments of these methods, less than a 12% decrease (e.g., less than a 10% decrease, less than a 9.5% decrease, less than a 9.0% decrease, less than a 8.5% decrease, less than a 8.0% decrease, less than a 7.5% decrease, less than a 7.0% decrease, less than a 6.5% decrease, less than a 6.0% decrease, less than a 5.5% decrease, less than a 5.0% decrease, less than a 4.5% decrease, less than a 4.0% decrease, less than a 3.5% decrease, less than a 3.0% decrease, less than a 2.5% decrease, less than a 2.0% decrease, less than a 1.5% decrease, less than a 1.0% decrease, less than a 0.5% decrease, or less than a 0.1% decrease) in cell density occurs following step (i) as compared to the composition prior to storage at a temperature of about 2° C. to about 10° C. (e.g., about 2° C. to about 9° C., about 2° C. to about 8° C., about 2° C. to about 7° C., about 2° C. to about 6° C., about 2° C. to about 5° C., about 2° C. to about 4° C., about 3° C. to about 10° C., about 3° C. to about 9° C., about 3° C. to about 8° C., about 3° C. to about 7° C., about 3° C. to about 6° C., about 3° C. to about 5° C., about 4° C. to about 10° C., about 4° C. to about 9° C., about 4° C. to about 8° C., about 4° C. to about 7° C., about 4° to about 6° C., about 5° C. to about 10° C., about 5° C. to about 9° C., about 5° C. to about 8° C., about 5° C. to about 7° C., about 6° C. to about 10° C., about 6° C. to about 9° C., about 6° C. to about 8° C., about 7° C. to about 10° C., about 7° C. to about 9° C., or about 8° C. to about 10° C.) for the period of time (e.g., any of the exemplary periods of time described herein, e.g., about 30 days to about 100 days, or any of the subranges of this range described herein).

Also provided herein are methods of treating a subject (e.g., any of the subjects described herein) that include administering any composition described herein to a subject in need thereof. In some embodiments of these methods, prior to the administering, the composition has been stored at a temperature of about 2° C. to about 10° C. (e.g., about 2° C. to about 9° C., about 2° C. to about 8° C., about 2° C. to about 7° C., about 2° C. to about 6° C., about 2° C. to about 5° C., about 2° C. to about 4° C., about 3° C. to about 10° C., about 3° C. to about 9° C., about 3° C. to about 8° C., about 3° C. to about 7° C., about 3° C. to about 6° C., about 3° C. to about 5° C., about 4° C. to about 10° C., about 4° C. to about 9° C., about 4° C. to about 8° C., about 4° C. to about 7° C., about 4° to about 6° C., about 5° C. to about 10° C., about 5° C. to about 9° C., about 5° C. to about 8° C., about 5° C. to about 7° C., about 6° C. to about 10° C., about 6° C. to about 9° C., about 6° C. to about 8° C., about 7° C. to about 10° C., about 7° C. to about 9° C., or about 8° C. to about 10° C.) for a period of time (e.g., any of the exemplary periods of time described herein, e.g., about 30 days to about 100 days, or any of the subranges of this range described herein), and less than 12% hemolysis (e.g., less than about 10% hemolysis, less than about 9.5% hemolysis, less than 9.0% hemolysis, less than 8.5% hemolysis, less than 8.0% hemolysis, less than 7.5% hemolysis, less than 7.0% hemolysis, less than 6.5% hemolysis, less than 6.0% hemolysis, less than 5.5% hemolysis, less than 5.0% hemolysis, less than 4.5% hemolysis, less than 4.0% hemolysis, less than 3.5% hemolysis, less than 3.0% hemolysis, less than 2.5% hemolysis, less than 2.0% hemolysis, less than 1.5% hemolysis, less than 1.0% hemolysis, less than 0.5% hemolysis, or less than 0.1% hemolysis) occurs following storage at a temperature of about 2° C. to about 10° C. for the period of time as compared to the composition prior to storage at a temperature of about 2° C. to about 10° C. for the period of time. In some embodiments of these methods, prior to the administering, the composition has been stored at a temperature of about 2° C. to about 10° C. (e.g., about 2° C. to about 9° C., about 2° C. to about 8° C., about 2° C. to about 7° C., about 2° C. to about 6° C., about 2° C. to about 5° C., about 2° C. to about 4° C., about 3° C. to about 10° C., about 3° C. to about 9° C., about 3° C. to about 8° C., about 3° C. to about 7° C., about 3° C. to about 6° C., about 3° C. to about 5° C., about 4° C. to about 10° C., about 4° C. to about 9° C., about 4° C. to about 8° C., about 4° C. to about 7° C., about 4° to about 6° C., about 5° C. to about 10° C., about 5° C. to about 9° C., about 5° C. to about 8° C., about 5° C. to about 7° C., about 6° C. to about 10° C., about 6° C. to about 9° C., about 6° C. to about 8° C., about 7° C. to about 10° C., about 7° C. to about 9° C., or about 8° C. to about 10° C.) for a period of time (e.g., any of the exemplary periods of time described herein, e.g., about 30 days to about 100 days, or any of the subranges of this range described herein), and less than a 12% decrease (e.g., less than a 10% decrease, less than a 9.5% decrease, less than a 9.0% decrease, less than a 8.5.% decrease, less than a 8.0% decrease, less than a 7.5% decrease, less than a 7.0% decrease, less than a 6.5% decrease, less than a 6.0% decrease, less than a 5.5% decrease, less than a 5.0% decrease, less than a 4.5% decrease, less than a 4.0% decrease, less than a 3.5% decrease, less than a 3.0% decrease, less than a 2.5% decrease, less than a 2.0% decrease, less than a 1.5% decrease, less than a 1.0% decrease, less than a 0.5% decrease, or less than a 0.1% decrease) in cell density occurs following storage at a temperature of about 2° C. to about 10° C. for the period of time as compared to the composition prior to storage at a temperature of about 2° C. to about 10° C. for the period of time.

Also provided are methods of making a composition, the methods include: (i) providing a population of enucleated erythroid cells; and (ii) resuspending the population of enucleated erythroid cells in a pharmaceutically acceptable aqueous buffered solution having a pH of about 6.5 to about 8.5 (e.g., any of the subranges of this range described herein) and an osmolarity of about 150 mOsm/L to about 400 mOsm/L (e.g., any of the subranges of this range described herein) that includes: about 5 mM to about 80 mM (e.g., any of the subranges of this range described herein) of a buffer (e.g., any of the exemplary buffers described herein or known in the art); about 5 mM to about 35 mM (e.g., any of the subranges of this range described herein) phosphate ion; about 50 mM to about 160 mM (e.g., any of the subranges of this range described herein) sodium ion; about 5 mM to about 60 mM (e.g., any of the subranges of this range described herein) potassium ion; about 0.01 mM to about 10 mM (e.g., any of the subranges of this range described herein) calcium ion; about 1 mM to about 20 mM (e.g., any of the subranges of this range described herein) magnesium ion; and about 5 mM to about 60 mM (e.g., any of the subranges of this range described herein) of a non-ionic cell impermeant agent (e.g., any of the exemplary non-ionic cell impermeant agents described herein or known in the art), where: the pharmaceutically acceptable aqueous buffered solution comprises less than 0.1 mM glucose (e.g., no detectable glucose); and optionally, the pharmaceutically acceptable aqueous buffered solution does not include one or more of: sucrose, a colloid, and an antioxidant. In some embodiments of these methods, the pharmaceutically acceptable aqueous buffered solution comprises less than 0.005 mM glucose, less than 0.001 mM glucose, no glucose, or no detectable glucose. In some embodiments of these methods, the pharmaceutically acceptable aqueous buffered solution does not include one or more (e.g., one, two, three, or four) of sucrose, a colloid (e.g., a dextran), and an antioxidant. In some embodiments of these methods, the storage of the composition at about 2° C. to about 10° C. (e.g., about 2° C. to about 9° C., about 2° C. to about 8° C., about 2° C. to about 7° C., about 2° C. to about 6° C., about 2° C. to about 5° C., about 2° C. to about 4° C., about 3° C. to about 10° C., about 3° C. to about 9° C., about 3° C. to about 8° C., about 3° C. to about 7° C., about 3° C. to about 6° C., about 3° C. to about 5° C., about 4° C. to about 10° C., about 4° C. to about 9° C., about 4° C. to about 8° C., about 4° C. to about 7° C., about 4° to about 6° C., about 5° C. to about 10° C., about 5° C. to about 9° C., about 5° C. to about 8° C., about 5° C. to about 7° C., about 6° C. to about 10° C., about 6° C. to about 9° C., about 6° C. to about 8° C., about 7° C. to about 10° C., about 7° C. to about 9° C., or about 8° C. to about 10° C.) for about 30 days to about 100 days (e.g., any of the subranges of this range described herein) results in less than 12% hemolysis (e.g., less than about 10% hemolysis, less than about 9.5% hemolysis, less than about 9.0% hemolysis, less than about 8.5% hemolysis, less than about 8.0% hemolysis, less than about 7.5% hemolysis, less than about 7.0% hemolysis, less than about 6.5% hemolysis, less than about 5.0% hemolysis, less than about 4.5% hemolysis, less than about 4.0% hemolysis, less than about 3.5% hemolysis, less than about 3.0% hemolysis, less than about 2.5% hemolysis, less than about 2.0% hemolysis, less than about 1.5% hemolysis, less than about 1.0% hemolysis, less than 0.5% hemolysis, or less than 0.1% hemolysis), e.g., as compared to prior to storage of the composition at about 2° C. to about 10° C. for about 30 days to about 100 days. In some embodiments of these methods, the storage of the composition at about 2° C. to about 10° C. (e.g., about 2° C. to about 9° C., about 2° C. to about 8° C., about 2° C. to about 7° C., about 2° C. to about 6° C., about 2° C. to about 5° C., about 2° C. to about 4° C., about 3° C. to about 10° C., about 3° C. to about 9° C., about 3° C. to about 8° C., about 3° C. to about 7° C., about 3° C. to about 6° C., about 3° C. to about 5° C., about 4° C. to about 10° C., about 4° C. to about 9° C., about 4° C. to about 8° C., about 4° C. to about 7° C., about 4° to about 6° C., about 5° C. to about 10° C., about 5° C. to about 9° C., about 5° C. to about 8° C., about 5° C. to about 7° C., about 6° C. to about 10° C., about 6° C. to about 9° C., about 6° C. to about 8° C., about 7° C. to about 10° C., about 7° C. to about 9° C., or about 8° C. to about 10° C.) for about 30 days to about 100 days (e.g., any of the subranges of this range described herein) results in less than a 12% in cell density (e.g., less than a 10% decrease, less than a 9.5% decrease, less than 9.0% hemolysis, less than 8.5% hemolysis, less than 8.0% hemolysis, less than 7.5% hemolysis, less than 7.0% hemolysis, less than 6.5% hemolysis, less than 6.0% hemolysis, less than 5.5% hemolysis, less than 5.0% hemolysis, less than 4.5% hemolysis, less than 4.0% hemolysis, less than 3.5% hemolysis, less than 3.0% hemolysis, less than 2.5% hemolysis, less than 2.0% hemolysis, less than 1.5% hemolysis, less than 1.0% hemolysis, less than 0.5% hemolysis, or less than 0.1% hemolysis) in cell density. Some embodiments of these methods further include culturing erythroid progenitor cells (e.g., any of the erythroid progenitor cells described herein) to provide the population of enucleated erythroid cells. An exemplary assay for measuring hemolysis is described herein. Also provided are compositions produced by any of the methods described herein.

Non-limiting aspects of these compositions and methods are described below. As can be appreciated by those in the field, the exemplary aspects listed below can be used in any combination, and can be combined with other aspects known in the field.

### Enucleated Erythroid Cells

In some embodiments of any of the compositions described herein, the composition comprises about 0.5×10⁸ to about 7.0×10⁹ enucleated erythroid cells/mL, e.g., about 0.5×10⁸ to about 6.0×10⁹, about 0.5×10⁸ to about 5.0×10⁹, about 0.5×10⁸ to about 4.0×10⁹, about 0.5×10⁸ to about 3.0×10⁹, about 0.5×10⁸ to about 2.0×10⁹, about 0.5×10⁸ to about 1.0×10⁹, about 0.5×10⁸ to about 0.5×10⁹, about 0.5×10⁸ to about 1.0×10⁸, about 1.0×10⁸ to about 7.0×10⁹, about 1.0×10⁸ to about 6.0×10⁹, about 1.0×10⁸ to about 5.0×10⁹, about 1.0×10⁸ to about 4.0×10⁹, about 1.0×10⁸ to about 3.0×10⁹, about 1.0×10⁸ to about 2.0×10⁹, about 1.0×10⁸ to about 1.0×10⁹, about 1.0×10⁸ to about 0.5×10⁹, about 0.5×10⁹ to about 7.0×10⁹, about 0.5×10⁹ to about 6.0×10⁹, about 0.5×10⁹ to about 5.0×10⁹, about 0.5×10⁹ to about 4.0×10⁹, about 0.5×10⁹ to about 3.0×10⁹, about 0.5×10⁹ to about 2.0×10⁹, about 0.5×10⁹ to about 1.0×10⁹, about 1.0×10⁹ to about 7.0×10⁹, about 1.0×10⁹ to about 6.0×10⁹, about 1.0×10⁹ to about 5.0×10⁹, about 1.0×10⁹ to about 4.0×10⁹, about 1.0×10⁹ to about 3.0×10⁹, about 1.0×10⁹ to about 2.0×10⁹, about 2.0×10⁹ to about 7.0×10⁹, about 2.0×10⁹ to about 6.0×10⁹, about 2.0×10⁹ to about 5.0×10⁹, about 2.0×10⁹ to about 4.0×10⁹, about 2.0×10⁹ to about 3.0×10⁹, about 3.0×10⁹ to about 7.0×10⁹, about 3.0×10⁹ to about 6.0×10⁹, about 3.0×10⁹ to about 5.0×10⁹, about 3.0×10⁹ to about 4.0×10⁹, about 4.0×10⁹ to about 7.0×10⁹, about 4.0×10⁹ to about 6.0×10⁹, about 4.0×10⁹ to about 5.0×10⁹, about 5.0×10⁹ to about 7.0×10⁹, about 5.0×10⁹ to about 6.0×10⁹, or about 6.0×10⁹ to about 7.0×10⁹ enucleated erythroid cells/mL.

In some embodiments, the enucleated erythroid cells (e.g., human enucleated erythroid cells) described herein are negative for (i.e., do not include) one or more minor blood group antigens, e.g., Le(a⁻b⁻) (for Lewis antigen system), Fy(a⁻b⁻) (for Duffy system), Jk(a⁻b⁻) (for Kidd system), M⁻N⁻ (for MNS system), K⁻k⁻ (for Kell system), Lu(a⁻b⁻) (for Lutheran system), and H-antigen negative (Bombay phenotype), or any combination thereof. In some embodiments, the enucleated erythroid cells are also Type O and/or Rh⁻. Minor blood groups are described, e.g., in Agarwal et al., “Blood group phenotype frequencies in blood donors from a tertiary care hospital in north India,” Blood Res. 48(1):51-54, 2013, and Mitra et al., “Blood groups systems,” Indian J. Anaesth. 58(5):524-528, 2014, the description of which is incorporated herein by reference.

In some embodiments, the enucleated erythroid cells (e.g., human enucleated erythroid cells) described herein exhibit substantially the same osmotic membrane fragility as an isolated, uncultured enucleated erythroid cell that does not comprise an exogenous protein (e.g., any of the exogenous proteins described herein or known in the art). In some embodiments, the population of enucleated erythroid cells has an osmotic fragility of less than 50% cell lysis at 0.3%, 0.35%, 0.4%, 0.45%, or 0.5% NaCl. Osmotic fragility is determined, in some embodiments, using the method described in Example 59 of WO 2015/073587 (the description of which is incorporated herein by reference).

In some embodiments, the enucleated erythroid cells (e.g., human enucleated erythroid cells) have approximately the same diameter or volume as a wild-type, untreated enucleated erythroid cell. In some embodiments, the population of enucleated erythroid cells (e.g., human enucleated erythroid cells) have an average diameter of about 4, 5, 6, 7, 8, 9, 10, 11 or 12 microns, or about 4.0 to about 12.0 microns, about 4.0 to about 11.5 microns, about 4.0 to about 11.0 microns, about 4.0 to about 10.5 microns, about 4.0 to about 10 microns, about 4.0 to about 9.5 microns, about 4.0 to about 9.0 microns, about 4.0 to about 8.5 microns, about 4.0 to about 8.0 microns, about 4.0 to about 7.5 microns, about 4.0 to about 7.0 microns, about 4.0 to about 6.5 microns, about 4.0 to about 6.0 microns, about 4.0 to about 5.5 microns, about 4.0 to about 5.0 microns, about 4.0 to about 4.5 microns, about 4.5 to about 12.0 microns, about 4.5 to about 11.5 microns, about 4.5 to about 11.0 microns, about 4.5 to about 10.5 microns, about 4.5 to about 10.0 microns, about 4.5 to about 9.5 microns, about 4.5 to about 9.0 microns, about 4.5 to about 8.5 microns, about 4.5 to about 8.0 microns, about 4.5 to about 7.5 microns, about 4.5 to about 7.0 microns, about 4.5 to about 6.5 microns, about 4.5 to about 6.0 microns, about 4.5 to about 5.5 microns, about 4.5 to about 5.0 microns, about 5.0 to about 12.0 microns, about 5.0 to about 11.5 microns, about 5.0 to about 11.0 microns, about 5.0 to about 10.5 microns, about 5.0 to about 10.0 microns, about 5.0 to about 9.5 microns, about 5.0 to about 9.0 microns, about 5.0 to about 8.5 microns, about 5.0 to about 8.0 microns, about 5.0 to about 7.5 microns, about 5.0 to about 7.0 microns, about 5.0 to about 6.5 microns, about 5.0 to about 6.0 microns, about 5.0 to about 5.5 microns, about 5.5 to about 12.0 microns, about 5.5 to about 11.5 microns, about 5.5 to about 11.0 microns, about 5.5 to about 10.5 microns, about 5.5 to about 10.0 microns, about 5.5 to about 9.5 microns, about 5.5 to about 9.0 microns, about 5.5 to about 8.5 microns, about 5.5 to about 8.0 microns, about 5.5 to about 7.5 microns, about 5.5 to about 7.0 microns, about 5.5 to about 6.5 microns, about 5.5 to about 6.0 microns, about 6.0 to about 12.0 microns, about 6.0 to about 11.5 microns, about 6.0 to about 11.0 microns, about 6.0 to about 10.5 microns, about 6.0 to about 10.0 microns, about 6.0 to about 9.5 microns, about 6.0 to about 9.0 microns, about 6.0 to about 8.5 microns, about 6.0 to about 8.0 microns, about 6.0 to about 7.5 microns, about 6.0 to about 7.0 microns, about 6.0 to about 6.5 microns, about 6.5 to about 12.0 microns, about 6.5 to about 11.5 microns, about 6.5 to about 11.0 microns, about 6.5 to about 10.5 microns, about 6.5 to about 10.0 microns, about 6.5 to about 9.5 microns, about 6.5 to about 9.0 microns, about 6.5 to about 8.5 microns, about 6.5 to about 8.0 microns, about 6.5 to about 7.5 microns, about 6.5 to about 7.0 microns, about 7.0 to about 12.0 microns, about 7.0 to about 11.5 microns, about 7.0 to about 11.0 microns, about 7.0 to about 10.5 microns, about 7.0 to about 10.0 microns, about 7.0 to about 9.5 microns, about 7.0 to about 9.0 microns, about 7.0 to about 8.5 microns, about 7.0 to about 8.0 microns, about 7.0 to about 7.5 microns, about 7.5 to about 12.0 microns, about 7.5 to about 11.5 microns, about 7.5 to about 11.0 microns, about 7.5 to about 10.5 microns, about 7.5 to about 10.0 microns, about 7.5 to about 9.5 microns, about 7.5 to about 9.0 microns, about 7.5 to about 8.5 microns, about 7.5 to about 8.0 microns, about 8.0 to about 12.0 microns, about 8.0 to about 11.5 microns, about 8.0 to about 11.0 microns, about 8.0 to about 10.5 microns, about 8.0 to about 10.0 microns, about 8.0 to about 9.5 microns, about 8.0 to about 9.0 microns, about 8.0 to about 8.5 microns, about 8.5 to about 12.0 microns, about 8.5 to about 11.5 microns, about 8.5 to about 11.0 microns, about 8.5 to about 10.5 microns, about 8.5 to about 10.0 microns, about 8.5 to about 9.5 microns, about 8.5 to about 9.0 microns, about 9.0 to about 12.0 microns, about 9.0 to about 11.5 microns, about 9.0 to about 11.0 microns, about 9.0 to about 10.5 microns, about 9.0 to about 10.0 microns, about 9.0 to about 9.5 microns, about 9.5 to about 12.0 microns, about 9.5 to about 11.5 microns, about 9.5 to about 11.0 microns, about 9.5 to about 10.5 microns, about 9.5 to about 10.0 microns, about 10.0 to about 12.0 microns, about 10.0 to about 11.5 microns, about 10.0 to about 11.0 microns, about 10.0 to about 10.5 microns, about 10.5 to about 12.0 microns, about 10.5 to about 11.5 microns, about 10.5 to about 11.0 microns, about 11.0 to about 12.0 microns, about 11.0 to about 11.5 microns, or about 11.5 to about 12.0 microns, and optionally the standard deviation of the population is less than 1, 2, or 3 microns. Enucleated erythroid cell diameter can be measured, e.g., using an Advia 120 hematology system, or a Moxi Z cell counter (Orflo).

In some embodiment the volume of the mean corpuscular volume of the enucleated erythroid cell is about 10 fL to about 175 fL, about 10 fL to about 160 fL, about 10 fL to about 140 fL, about 10 fL to about 120 fL, about 10 fL to about 100 fL, about 10 fL to about 95 fL, about 10 fL to about 90 fL, about 10 fL to about 85 fL, about 10 fL to about 80 fL, about 10 fL to about 75 fL, about 10 fL to about 70 fL, about 10 fL to about 65 fL, about 10 fL to about 60 fL, about 10 fL to about 55 fL, about 10 fL to about 50 fL, about 10 fL to about 45 fL, about 10 fL to about 40 fL, about 10 fL to about 35 fL, about 10 fL to about 30 fL, about 10 fL to about 25 fL, about 10 fL to about 20 fL, about 10 fL to about 15 fL, about 15 fL to about 175 fL, about 15 fL to about 160 fL, about 15 fL to about 140 fL, about 15 fL to about 120 fL, about 15 fL to about 100 fL, about 15 fL to about 95 fL, about 15 fL to about 90 fL, about 15 fL to about 85 fL, about 15 fL to about 80 fL, about 15 fL to about 75 fL, about 15 fL to about 70 fL, about 15 fL to about 65 fL, about 15 fL to about 60 fL, about 15 fL to about 55 fL, about 15 fL to about 50 fL, about 15 fL to about 45 fL, about 15 fL to about 40 fL, about 15 fL to about 35 fL, about 15 fL to about 30 fL, about 15 fL to about 25 fL, about 15 fL to about 20 fL, about 20 fL to about 175 fL, about 20 fL to about 160 fL, about 20 fL to about 140 fL, about 20 fL to about 120 fL, about 20 fL to about 100 fL, about 20 fL to about 95 fL, about 20 fL to about 90 fL, about 20 fL to about 85 fL, about 20 fL to about 80 fL, about 20 fL to about 75 fL, about 20 fL to about 70 fL, about 20 fL to about 65 fL, about 20 fL to about 60 fL, about 20 fL to about 55 fL, about 20 fL to about 50 fL, about 20 fL to about 45 fL, about 20 fL to about 40 fL, about 20 fL to about 35 fL, about 20 fL to about 30 fL, about 20 fL to about 25 fL, about 25 fL to about 175 fL, about 25 fL to about 160 fL, about 25 fL to about 140 fL, about 25 fL to about 120 fL, about 25 fL to about 100 fL, about 25 fL to about 95 fL, about 25 fL to about 90 fL, about 25 fL to about 85 fL, about 25 fL to about 80 fL, about 25 fL to about 75 fL, about 25 fL to about 70 fL, about 25 fL to about 65 fL, about 25 fL to about 60 fL, about 25 fL to about 55 fL, about 25 fL to about 50 fL, about 25 fL to about 45 fL, about 25 fL to about 40 fL, about 25 fL to about 35 fL, about 25 fL to about 30 fL, about 30 fL to about 175 fL, about 30 fL to about 160 fL, about 30 fL to about 140 fL, about 30 fL to about 120 fL, about 30 fL to about 100 fL, about 30 fL to about 95 fL, about 30 fL to about 90 fL, about 30 fL to about 85 fL, about 30 fL to about 80 fL, about 30 fL to about 75 fL, about 30 fL to about 70 fL, about 30 fL to about 65 fL, about 30 fL to about 60 fL, about 30 fL to about 55 fL, about 30 fL to about 50 fL, about 30 fL to about 45 fL, about 30 fL to about 40 fL, about 30 fL to about 35 fL, about 35 fL to about 175 fL, about 35 fL to about 160 fL, about 35 fL to about 140 fL, about 35 fL to about 120 fL, about 35 fL to about 100 fL, about 35 fL to about 95 fL, about 35 fL to about 90 fL, about 35 fL to about 85 fL, about 35 fL to about 80 fL, about 35 fL to about 75 fL, about 35 fL to about 70 fL, about 35 fL to about 65 fL, about 35 fL to about 60 fL, about 35 fL to about 55 fL, about 35 fL to about 50 fL, about 35 fL to about 45 fL, about 35 fL to about 40 fL, about 40 fL to about 175 fL, about 40 fL to about 160 fL, about 40 fL to about 140 fL, about 40 fL to about 120 fL, about 40 fL to about 100 fL, about 40 fL to about 95 fL, about 40 fL to about 90 fL, about 40 fL to about 85 fL, about 40 fL to about 80 fL, about 40 fL to about 75 fL, about 40 fL to about 70 fL, about 40 fL to about 65 fL, about 40 fL to about 60 fL, about 40 fL to about 55 fL, about 40 fL to about 50 fL, about 40 fL to about 45 fL, about 45 fL to about 175 fL, about 45 fL to about 160 fL, about 45 fL to about 140 fL, about 45 fL to about 120 fL, about 45 fL to about 100 fL, about 45 fL to about 95 fL, about 45 fL to about 90 fL, about 45 fL to about 85 fL, about 45 fL to about 80 fL, about 45 fL to about 75 fL, about 45 fL to about 70 fL, about 45 fL to about 65 fL, about 45 fL to about 60 fL, about 45 fL to about 55 fL, about 45 fL to about 50 fL, about 50 fL to about 175 fL, about 50 fL to about 160 fL, about 50 fL to about 140 fL, about 50 fL to about 120 fL, about 50 fL to about 100 fL, about 50 fL to about 95 fL, about 50 fL to about 90 fL, about 50 fL to about 85 fL, about 50 fL to about 80 fL, about 50 fL to about 75 fL, about 50 fL to about 70 fL, about 50 fL to about 65 fL, about 50 fL to about 60 fL, about 50 fL to about 55 fL, about 60 fL to about 175 fL, about 60 fL to about 160 fL, about 60 fL to about 140 fL, about 60 fL to about 120 fL, about 60 fL to about 100 fL, about 60 fL to about 95 fL, about 60 fL to about 90 fL, about 60 fL to about 85 fL, about 60 fL to about 80 fL, about 60 fL to about 75 fL, about 60 fL to about 70 fL, about 60 fL to about 65 fL, about 70 fL to about 175 fL, about 70 fL to about 160 fL, about 70 fL to about 140 fL, about 70 fL to about 120 fL, about 70 fL to about 100 fL, about 70 fL to about 95 fL, about 70 fL to about 90 fL, about 70 fL to about 85 fL, about 70 fL to about 80 fL, about 70 fL to about 75 fL, about 80 fL to about 175 fL, about 80 fL to about 160 fL, about 80 fL to about 140 fL, about 80 fL to about 120 fL, about 80 fL to about 100 fL, about 80 fL to about 95 fL, about 80 fL to about 90 fL, about 80 fL to about 85 fL, about 100 fL to about 175 fL, about 100 fL to about 160 fL, about 100 fL to about 140 fL, about 100 fL to about 120 fL, about 120 fL to about 175 fL, about 120 fL to about 160 fL, about 120 fL to about 140 fL, about 140 fL to about 175 fL, about 140 fL to about 160 fL, or about 160 fL to about 175 fL, and optionally, the standard deviation of the population is less than 50, 40, 30, 20, 10, 5, or 2 fL. The mean corpuscular volume can be measured, e.g., using a hematological analysis instrument, e.g., a Coulter counter, a Moxi Z cell counter (Orflo), or a Sysmex Hematology analyzer.

In some embodiments of any of the compositions described herein, the enucleated erythroid cells are human (e.g., derived from a human donor erythroid progenitor cell) enucleated erythroid cells.

In some embodiments of any of the compositions described herein, the enucleated erythroid cells are engineered human enucleated erythroid cells. In some examples, the engineered enucleated erythroid cells comprise a single exogenous protein (e.g., an exogenous protein present in the cytosol or present on the membrane of the engineered enucleated erythroid cell) (e.g., any of the exemplary exogenous proteins described herein or known in the art).

In other examples, the engineered enucleated erythroid cells comprise two or more exogenous proteins (e.g., any of the exemplary exogenous proteins described herein). In some examples, at least one of the two or more exogenous proteins can be present in the cytosol of the engineered enucleated erythroid cell (e.g., an enzyme, e.g., phenylalanine ammonia lyase). In some examples, at least one of the two or more exogenous proteins can be present on the membrane of the engineered enucleated erythroid cell (e.g., an Fc-binding molecule, a cytokine receptor, T-cell activating ligands, T-cell receptors, immune inhibitory molecules, WIC molecules, APC-binding molecules, autoantigens, allergens, toxins, targeting agents, receptor ligands (e.g., receptor agonists or receptor antagonists), or antibodies or antibody fragments).

Non-limiting examples of the one or more exogenous proteins that any of the engineered erythroid cells described herein can comprise are listed below in Tables A-D, in addition to the corresponding disease or condition that an engineered erythroid cell comprising the exogenous protein can be used to treat. Additional examples of exogenous proteins that can be comprised by any of the erythroid cells described herein are known in the art.

TABLE A
Exemplary Exogenous Proteins

| Genus | Exogenous Protein | Disease or treatment |
|---|---|---|
| Enzymes | Phenylalanine ammonia | Phenylketonuria (PKU); method |
| | lyase (PAL) (e.g., | of reducing phenylalanine in the |
| | Anabaena variabilis PAL) | blood of a subject |
| | Phenylalanine | Phenylketonuria (PKU); method |
| | hydroxylase (PAH) | of reducing phenylalanine in the |
| | | blood of a subject |
| | Asparaginase | Cancer |
| | Glutaminase | Cancer |
| | Cystathionine gamma | Homocystinuria; method of |
| | lyase (CGL) | reducing homocysteine levels in |
| | | the blood of a subject |
| | Uricase | Hyperuricemia, rheumatoid |
| | | arthritis, osteoarthritis, cerebral |
| | | stroke, ischemic heart disease, |
| | | arrhythmia, and chronic renal |
| | | disease |
| | Cystathionine beta | Homocystinuria; method of |
| | synthase (CBS) | reducing homocysteine levels in |
| | | the blood of a subject |
| | Oxalate oxidase (OxOx) | hyperoxaluria; method of |
| | | reducing the level of oxalate |
| | Oxalate decarboxylase | (OxDC) hyperoxaluria; method |
| | | of reducing the level of oxalate |

TABLE B
Exemplary Exogenous Proteins

| Genus | Exogenous Protein |
|---|---|
| Antigens | CD19, CD20, CD123, CD33, CD133, CD138, CD5, CD7, |
| | CD22, CD30, myelin basic protein, myelin proteolipid |
| | protein, myelin oligodendrocyte glycoprotein (MOG), |
| | phospholipase A2 receptor, beta-2 glycoprotein 1, a tumor |
| | antigen or neoantigen (e.g., a melanoma antigen genes-A |
| | (MAGE-A) antigen or a p53 peptide) an autoimmune |
| | disease antigen, a viral antigen (e.g., an Epstein barr |
| | virus (EBV) antigen, a human papilloma virus (HPV) |
| | antigen, and a hepatitis B virus (HBV) antigen), a |
| | bacterial antigen, or a parasite antigen; a neutrophil |
| | granule protease antigen, a NY-ESO-1/LAGE-2 antigen, |
| | a telomerase antigen, a glycoprotein 100 (gp100) antigen |

TABLE C
Exemplary Exogenous Proteins

| Genus | Exogenous Protein | Disease or treatment |
|---|---|---|
| Immuno- | cytokines, interleukins, cytokine | Cancer, |
| modulatory | receptors, Fc-binding | autoimmune |
| Molecules | molecules, T-cell activating | disorders |
| | ligands, T cell receptors, | |
| | immune inhibitory molecules, | |
| | costimulatory molecule, | |
| | coinhibitory molecules (e.g., IL- | |
| | 35, IL-10, VSIG-3 or a LAG3 | |
| | agonist), MHC molecules, APC- | |
| | binding molecules, TRAIL | |
| | receptor ligands. | |
| | Exemplary immunomodulatory | |
| | molecules include, 4-1BBL, | |
| | LIGHT, anti CD28, CD80, CD86, | |
| | CD70, OX40L, GITRL, TIM4, | |
| | SLAM, CD48, CD58, CD83, | |
| | CD155, CD112, IL-15, IL-15Rα | |
| | fused to IL-15, IL-21, ICAM-1, a | |
| | ligand for LFA-1, anti-CD3, IL2, | |
| | IL15, 15Rα fused to IL-15, IL7, | |
| | IL12, IL18, IL21, IL4, IL6, IL23, | |
| | IL27, IL17, ILID, TGF-beta, IFN- | |
| | gamma, IL-1 beta, GM-CSF, and | |
| | IL-25. | |
| | Exemplary combination: IL- | |
| | 15Rα fused to IL-15 and 4-1BBL | |
| | Exemplary Combination: IL-12 | |
| | and 4-1BBL | |

TABLE D
Exemplary Exogenous Proteins

| | |
|---|---|
| Antigen Presenting | an MHC class I polypeptide, an MHC class I single chain fusion |
| Molecule | protein, an MHC class II polypeptide, or an MHC class II single |
| | chain fusion protein |
| | Either unbound or bound (e.g., covalently or as a fusion protein) to |
| | an antigen |
| preproinsulin, proinsulin, | Diabetes |
| insulin | |

In some embodiments, an exogenous protein present on the membrane of the engineered enucleated erythroid cell can be a product of a click chemistry reaction (e.g., the exogenous protein may be conjugated to a protein present on the membrane of the cell (e.g., a second exogenous protein or an endogenous protein) using any of the methods described herein). In some embodiments, an exogenous protein present on the membrane of the engineered enucleated erythroid cell can the a product of a conjugation reaction using a sortase enzyme (e.g., the exogenous protein may be conjugated to a protein present on the membrane of the cell (e.g., a second exogenous protein or an endogenous protein) using any of the methods described herein). Non-limiting examples of a conjugation reaction using a sortase enzyme can be found in U.S. Pat. No. 10,260,038 and U.S. Pat. Pub. No. 2016/0082046 A1. In some embodiments, an exogenous protein present on the membrane of the engineered enucleated erythroid cell can be a lipid-anchored protein, e.g., a GPI-anchor, an N-myristolyated protein, or a S-palmitoylated protein. In some embodiments an exogenous protein present on the membrane of the engineered enucleated erythroid cell can be a transmembrane protein (e.g., a single-pass or multi-pass transmembrane protein) or a peripheral membrane protein. In some embodiments, an exogenous protein present on the membrane of the engineered enucleated erythroid cell can be a fusion protein comprising a transmembrane domain (e.g., a fusion protein comprising the transmembrane domain of small integral membrane protein 1 (SMIM1) or glycophorin A (GPA)). In some embodiments, an exogenous protein present on the membrane of the engineered enucleated erythroid cell does not have any amino acids protruding into the extracellular space. In some embodiments, an exogenous protein present on the membrane of the engineered enucleated erythroid cell does not have any amino acids protruding into the cytosol of the engineered enucleated erythroid cell. In some embodiments, an exogenous protein present on the membrane of the engineered enucleated erythroid cell has amino acids protruding into the extracellular space and amino acids protruding into the cytosol of the engineered enucleated erythroid cells.

The engineered enucleated erythroid cells can be produced by introducing one or more nucleic acids (e.g., DNA expression vectors or mRNA) encoding one or more exogenous proteins (e.g., any of the exogenous proteins described herein or known in the art) into an erythroid progenitor cell (e.g., any of the erythroid progenitor cells described herein or known in the art). Exemplary methods for introducing DNA expression vectors into erthyroid progenitor cells include, but are not limited to, liposome-mediated transfer, transformation, gene guns, transfection, and transduction, e.g., viral-mediated gene transfer (e.g., performed using viral vectors including adenovirus vectors, adeno-associated viral vectors, lentiviral vectors, herpes viral vectors, and retroviral-based vectors). Additional exemplary methods for introducing DNA expression vectors into erythroid progenitor cells include the use of, e.g., naked DNA, CaPO₄ precipitation, DEAE dextran, electroporation, protoplast fusion, lipofection, and cell microinjection.

An erythroid progenitor cell can optionally be cultured, e.g., before and/or after introduction of one or more nucleic acids encoding one or more exogenous proteins, under suitable conditions allowing for differentiation into engineered enucleated erythroid cells. In some embodiments, the resulting engineered enucleated erythroid cells comprise proteins associated with mature erythrocytes, e.g., hemoglobin (e.g., adult hemoglobin and/or fetal hemoglobin), glycophorin A, and exogenous proteins which can be validated and quantified by standard methods (e.g. Western blotting or FACS analysis).

In some examples, enucleated erythroid cells or erythroid progenitor cells can be transfected with mRNA encoding an exogenous protein to generate engineered enucleated erythroid cells. Messenger RNA can be derived from in vitro transcription of a cDNA plasmid construct containing a sequence encoding an exogenous protein. For example, the cDNA sequence encoding an exogenous protein may be inserted into a cloning vector containing a promoter sequence compatible with specific RNA polymerases. For example, the cloning vector ZAP Express® pBK-CMV (Stratagene, La Jolla, Calif., USA) contains T3 and T7 promoter sequences compatible with the T3 and T7 RNA polymerases, respectively. For in vitro transcription of sense mRNA, the plasmid is linearized at a restriction site downstream of the stop codon(s) corresponding to the end of the sequence encoding the exogenous protein. The mRNA is transcribed from the linear DNA template using a commercially available kit such as, for example, the RNAMaxx® High Yield Transcription Kit (from Stratagene, La Jolla, Calif., USA). In some instances, it may be desirable to generate 5′-m7GpppG-capped mRNA. As such, transcription of a linearized cDNA template may be carried out using, for example, the mMESSAGE mMACHINE High Yield Capped RNA Transcription Kit from Ambion (Austin, Tex., USA). Transcription may be carried out in a reaction volume of 20-100 μl at 37° C. for 30 min to 4 h. The transcribed mRNA is purified from the reaction mix by a brief treatment with DNase I to eliminate the linearized DNA template followed by precipitation in 70% ethanol in the presence of lithium chloride, sodium acetate, or ammonium acetate. The integrity of the transcribed mRNA may be assessed using electrophoresis with an agarose-formaldehyde gel or commercially available Novex pre-cast TBE gels (Novex, Invitrogen, Carlsbad, Calif., USA).

Messenger RNA encoding an exogenous protein may be introduced into enucleated erythroid cells or erythroid progenitor cells using a variety of approaches including, for example, lipofection and electroporation (van Tandeloo et al., Blood 98:49-56, 2001). For lipofection, for example, 5 μg of in vitro transcribed mRNA in Opti-MEM (Invitrogen, Carlsbad, Calif., USA) is incubated for 5-15 min at a 1:4 ratio with the cationic lipid DMRIE-C (Invitrogen).

Alternatively, a variety of other cationic lipids or cationic polymers may be used to transfect erythroid progenitor cells with mRNA including, for example, DOTAP, various forms of polyethylenimine, and polyL-lysine (Sigma-Aldrich, Saint Louis, Mo., USA), and Superfect (Qiagen, Inc., Valencia, Calif., USA; See, e.g., Bettinger et al., Nucleic Acids Res. 29:3882-3891, 2001). The resulting mRNA/lipid complexes are incubated with cells (1-2×10⁶ cells/mL) for 2 hours at 37° C., washed, and returned to culture. For electroporation, for example, about 5 to 20×10⁶ cells in 500 μL of Opti-MEM (Invitrogen, Carlsbad, Calif., USA) are mixed with about 20 μg of in vitro transcribed mRNA and electroporated in a 0.4-cm cuvette using, for example, an Easyject Plus device (EquiBio, Kent, United Kingdom). In some instances, it may be necessary to test various voltages, capacitances, and electroporation volumes to determine the useful conditions for transfection of a particular mRNA into an erythroid progenitor cell. In general, the electroporation parameters required to efficiently transfect cells with mRNA appear to be less detrimental to cells than those required for electroporation of DNA (van Tandeloo et al., Blood 98:49-56, 2001).

Alternatively, mRNA may be transfected into enucleated erythroid cells or erythroid progenitor cells using a peptide-mediated RNA delivery strategy (See, e.g., Bettinger et al., Nucleic Acids Res. 29:3882-3891, 2001). For example, the cationic lipid polyethylenimine 2 kDA (Sigma-Aldrich, Saint Louis, Mo., USA) may be combined with the melittin peptide (Alta Biosciences, Birmingham, UK) to increase the efficiency of mRNA transfection, particularly in postmitotic primary cells. The mellitin peptide may be conjugated to the PEI using a disulfide cross-linker such as, for example, the hetero-bifunctional cross-linker succinimidyl 3-(2-pyridyldithio) propionate. In vitro transcribed mRNA is preincubated for 5 to 15 min with the mellitin-PEI to form an RNA/peptide/lipid complex. This complex is then added to cells in serum-free culture medium for 2 to 4 h at 37° C. in a 5% CO₂ humidified environment, then removed, and the transfected cells further cultured.

In some embodiments, the engineered enucleated erythroid cells are generated by introducing a nucleic acid (e.g., any of the exemplary nucleic acids described herein) encoding one or more exogenous protein(s) (e.g., any exogenous protein or any combination of exogenous proteins described herein) into an erythroid progenitor cell. In some embodiments, the exogenous protein is encoded by a DNA, which is introduced into an erythroid progenitor cell. In some embodiments, the exogenous protein is encoded by an RNA, which is introduced into an erythroid progenitor cell.

Nucleic acid encoding one or more exogenous protein(s) may be introduced into an erythroid progenitor cell prior to terminal differentiation into an enucleated erythroid cell using a variety of DNA techniques, including, e.g., transient or stable transfections and gene therapy approaches.

Viral gene transfer may be used to transfect the cells with a nucleic acid encoding one or more exogenous protein(s). A number of viruses may be used as gene transfer vehicles including Moloney murine leukemia virus (MMLV), adenovirus, adeno-associated virus (AAV), herpes simplex virus (HSV), lentiviruses such as human immunodeficiency virus 1 (HIV1), and spumaviruses such as foamy viruses (see, e.g., Osten et al., HEP 178:177-202, 2007). Retroviruses, for example, efficiently transduce mammalian cells including human cells and integrate into chromosomes, conferring stable gene transfer.

A nucleic acid encoding one or more exogenous protein(s) can be transfected into an erythroid progenitor cell. A suitable vector is the Moloney murine leukemia virus (MMLV) vector (Malik et al., Blood 91:2664-2671, 1998). Vectors based on MMLV, an oncogenic retrovirus, are currently used in gene therapy clinical trials (Hassle et al., News Physiol. Sci. 17:87-92, 2002). For example, a DNA construct containing the cDNA encoding an exogenous protein can be generated in the MMLV vector backbone using standard molecular biology techniques. The construct is transfected into a packaging cell line such as, for example, PA317 cells and the viral supernatant is used to transfect producer cells such as, for example, PG13 cells. The PG13 viral supernatant is incubated with an erythroid progenitor cell. The expression of the exogenous protein may be monitored using FACS analysis (fluorescence-activated cell sorting), for example, with a fluorescently labeled antibody directed against the exogenous protein, if it is present on the membrane of the engineered human enucleated erythroid cell. Similar methods may be used such that an exogenous protein is present in the cytosol of an engineered human enucleated erythroid cell.

Optionally, a nucleic acid encoding a fluorescent tracking molecule such as, for example, green fluorescent protein (GFP), can be transfected into an erythroid progenitor cell using a viral-based approach (Tao et al., Stem Cells 25:670-678, 2007). Ecotopic retroviral vectors containing DNA encoding the enhanced green fluorescent protein (EGFP) or a red fluorescent protein (e.g., DsRed-Express) are packaged using a packaging cell such as, for example, the Phoenix-Eco cell line (distributed by Orbigen, San Diego, Calif.). Packaging cell lines stably express viral proteins needed for proper viral packaging including, for example, gag, pol, and env. Supernatants from the Phoenix-Eco cells into which viral particles have been shed are used to transduce erythroid progenitor cells. In some instances, transduction may be performed on a specially coated surface such as, for example, fragments of recombinant fibronectin to improve the efficiency of retroviral mediated gene transfer (e.g., RetroNectin, Takara Bio USA, Madison, Wis.). Cells are incubated in RetroNectin-coated plates with retroviral Phoenix-Eco supernatants plus suitable co-factors. Transduction may be repeated the next day. In this instance, the percentage of erythroid progenitor cells expressing EGFP or DsRed-Express may be assessed by FACS. Other reporter genes that may be used to assess transduction efficiency include, for example, beta-galactosidase, chloramphenicol acetyltransferase, and luciferase, as well as low-affinity nerve growth factor receptor (LNGFR), and the human cell surface CD24 antigen (Bierhuizen et al., Leukemia 13:605-613, 1999).

Nonviral vectors may be used to introduce a nucleic acid encoding one or more exogenous protein(s) into an erythroid progenitor cell to generate engineered enucleated erythroid cells. A number of delivery methods can be used to introduce nonviral vectors into erythroid progenitor cells including chemical and physical methods.

A nonviral vector encoding an exogenous protein may be introduced into an erythroid progenitor cell using synthetic macromolecules, such as cationic lipids and polymers (Papapetrou et al., Gene Therapy 12:S118-S130, 2005). Cationic liposomes, for example form complexes with DNA through charge interactions. The positively charged DNA/lipid complexes bind to the negative cell surface and are taken up by the cell by endocytosis. This approach may be used, for example, to transfect hematopoietic cells (see, e.g., Keller et al., Gene Therapy 6:931-938, 1999). For erythroid progenitor cells, the plasmid DNA (in a serum-free medium, such as, for example, OptiMEM (Invitrogen, Carlsbad, Calif.)) is mixed with a cationic liposome (in serum free medium), such as the commercially available transfection reagent Lipofectamine™ (Invitrogen, Carlsbad, Calif.), and allowed to incubate for at least 20 minutes to form complexes. The DNA/liposome complex is added to erythroid progenitor cells and allowed to incubate for 5-24 h, after which time transgene expression of the exogenous protein(s) may be assayed. Alternatively, other commercially available liposome tranfection agents may be used (e.g., In vivo GeneSHUTTLE™, Qbiogene, Carlsbad, Calif.).

Optionally, a cationic polymer such as, for example, polyethylenimine (PEI) may be used to efficiently transfect erythroid progenitor cells, for example hematopoietic and umbilical cord blood-derived CD34⁺ cells (see, e.g., Shin et al., Biochim. Biophys. Acta 1725:377-384, 2005). Human CD34⁺ cells are isolated from human umbilical cord blood and cultured in Iscove's modified Dulbecco's medium supplemented with 200 ng/ml stem cell factor and 20% heat-inactivated serum. Plasmid DNA encoding the exogenous protein(s) is incubated with branched or linear PEIs varying in size from 0.8 K to 750 K (Sigma Aldrich, Saint Louis, Mo., USA; Fermetas, Hanover, Md., USA). PEI is prepared as a stock solution at 4.2 mg/mL distilled water and slightly acidified to pH 5.0 using HCl. The DNA may be combined with the PEI for 30 min at room temperature at various nitrogen/phosphate ratios based on the calculation that 1 μg of DNA contains 3 nmol phosphate and 1 μL of PEI stock solution contains 10 nmol amine nitrogen. The isolated CD34⁺ cells are seeded with the DNA/cationic complex, centrifuged at 280×g for 5 minutes and incubated in culture medium for 4 or more hours until expression of the exogenous protein(s) is/are assessed.

A plasmid vector may be introduced into suitable erythroid progenitor cells using a physical method such as particle-mediated transfection, “gene gun,” biolistics, or particle bombardment technology (Papapetrou, et al., Gene Therapy 12:S118-S130, 2005). In this instance, DNA encoding the exogenous protein is absorbed onto gold particles and administered to cells by a particle gun. This approach may be used, for example, to transfect erythroid progenitor cells, e.g., hematopoietic stem cells derived from umbilical cord blood (See, e.g., Verma et al., Gene Therapy 5:692-699, 1998). As such, umbilical cord blood is isolated and diluted three-fold in phosphate buffered saline. CD34⁺ cells are purified using an anti-CD34 monoclonal antibody in combination with magnetic microbeads coated with a secondary antibody and a magnetic isolation system (e.g., Miltenyi MiniMac System, Auburn, Calif., USA). The CD34⁺ enriched cells may be cultured as described herein. For transfection, plasmid DNA encoding the exogenous protein(s) is precipitated onto a particle, e.g., gold beads, by treatment with calcium chloride and spermidine. Following washing of the DNA-coated beads with ethanol, the beads may be delivered into the cultured cells using, for example, a Biolistic PDS-1000/He System (Bio-Rad, Hercules, Calif., USA). A reporter gene such as, for example, beta-galactosidase, chloramphenicol acetyltransferase, luciferase, or green fluorescent protein may be used to assess efficiency of transfection.

Optionally, electroporation methods may be used to introduce a plasmid vector into erythroid progenitor cells. Electroporation creates transient pores in the cell membrane, allowing for the introduction of various molecules into the cells including, for example, DNA and RNA. As such, CD34⁺ cells are isolated and cultured as described herein. Immediately prior to electroporation, the cells are isolated by centrifugation for 10 min at 250×g at room temperature and resuspended at 0.2-10×10⁶ viable cells/ml in an electroporation buffer such as, for example, X-VIVO 10 supplemented with 1.0% human serum albumin (HSA). The plasmid DNA (1-50 μg) is added to an appropriate electroporation cuvette along with 500 μL of cell suspension.

Electroporation may be done using, for example, an ECM 600 electroporator (Genetronics, San Diego, Calif., USA) with voltages ranging from 200 V to 280 V and pulse lengths ranging from 25 to 70 milliseconds. A number of alternative electroporation instruments are commercially available and may be used for this purpose (e.g., Gene Pulser Xcell™, BioRad, Hercules, Calif.; Cellject Duo, Thermo Science, Milford, Mass.). Alternatively, efficient electroporation of isolated CD34⁺ cells may be performed using the following parameters: 4 mm cuvette, 1600 μE, 550 V/cm, and 10 μg of DNA per 500 μL of cells at 1×10⁵ cells/mL (Oldak et al., Acta Biochim. Polonica 49:625-632, 2002).

Nucleofection, a form of electroporation, may also be used to transfect erythroid progenitor cells. In this instance, transfection is performed using electrical parameters in cell-type specific solutions that enable DNA (or other reagents) to be directly transported to the nucleus, thus reducing the risk of possible degradation in the cytoplasm. For example, a Human CD34 Cell Nucleofector™ Kit (from Amaxa Inc.) may be used to transfect erythroid progenitor cells. In this instance, 1-5×10⁶ cells in Human CD34 Cell Nucleofector™ Solution are mixed with 1-5 μg of DNA and transfected in the Nucleofector™ instrument using preprogrammed settings as determined by the manufacturer.

Erythroid progenitor cells may be non-virally transfected with a conventional expression vector which is unable to self-replicate in mammalian cells unless it is integrated in the genome. Alternatively, erythroid progenitor cells may be transfected with an episomal vector which may persist in the host nucleus as autonomously replicating genetic units without integration into chromosomes (Papapetrou et al., Gene Therapy 12:S118-S130, 2005). These vectors exploit genetic elements derived from viruses that are normally extrachromosomally replicating in cells upon latent infection such as, for example, EBV, human polyomavirus BK, bovine papilloma virus-1 (BPV-1), herpes simplex virus-1 (HSV), and Simian virus 40 (SV40). Mammalian artificial chromosomes may also be used for nonviral gene transfer (Vanderbyl et al., Exp. Hematol. 33:1470-1476, 2005).

Exogenous nucleic acid encoding one or more exogenous protein(s) can be assembled into expression vectors by standard molecular biology methods known in the art, e.g., restriction digestion, overlap-extension PCR, and Gibson assembly.

Exogenous nucleic acids can comprise a gene encoding an exogenous protein that is not normally present on the cell surface, e.g., of an enucleated erythroid cell, fused to a gene that encodes an endogenous or native membrane protein, such that the exogenous protein is expressed on the cell surface. For example, an exogenous gene encoding an exogenous protein can be cloned at the N terminus following the leader sequence of a type 1 membrane protein, at the C terminus of a type 2 membrane protein, or upstream of the GPI attachment site of a GPI-linked membrane protein.

Standard cloning methods can be used to introduce flexible amino acid linkers between two fused genes. For example, the flexible linker is a poly-glycine poly-serine linker such as [Gly₄Ser]₃ (SEQ ID NO: 1) commonly used in generating single-chain antibody fragments from full-length antibodies (Antibody Engineering: Methods & Protocols, B. Lo, ed., Humana Press, 2004, 576 pp.), or Ala-Gly-Ser-Thr polypeptides such as those used to generate single-chain Arc repressors (Robinson & Sauer, Proc. Nat'l. Acad. Sci. U.S.A. 95: 5929-34, 1998). In some embodiments, the flexible linker provides the exogenous protein with more flexibility and steric freedom than the equivalent construct without the flexible linker. This added flexibility is useful in applications that require binding to a target, e.g., an antibody or protein, or an enzymatic reaction of the protein for which the active site must be accessible to the substrate (e.g., the target).

In some embodiments, the methods provided include the delivery of large nucleic acids (specifically RNAs, such as mRNA) into erythroid progenitor cells by contacting the erythroid progenitor cell with the nucleic acid and introducing the nucleic acid by electroporation under conditions effective for delivery of the nucleic acid to the cell, such as those described herein. Suitable electroporators include, but are not limited to, the Bio-Rad GENE PULSER and GENE PULSER II; the Life Technologies NEON; BTX GEMINI system; and MAXCYTE electroporator. These methods do not require viral delivery or the use of viral vectors. Suitable nucleic acids include RNAs, such as mRNAs. Suitable nucleic acids also include DNAs, including transposable elements, stable episomes, plasmid DNA, or linear DNA.

Conditions for the electroporation of cell lines have been described in the literature, e.g. by Van Tendeloo et al., Blood 98(1):49-56, 2001. Suitable electroporation conditions for the methods described herein include for a Life Technologies Neon Transfection System: a pulse voltage ranging from about 500 to about 2000 V, from about 800 to about 1800 V, or from about 850 to about 1700 V; a pulse width ranging from about 5 to about 50 msec, or from about 10 to about 40 msec; and a pulse number ranging from 1 to 2 pulses, 1 to 3 pulses, 1 to 4 pulses, or 1 to 5 pulses.

Particularly suitable conditions for electroporation of erythroid progenitor cells include, e.g., for 4 days: a) pulse voltage 1300-1400, pulse width: 10-20 msec, number of pulses: 1-3; b) pulse voltage 1400, pulse width: 10 msec, number of pulses: 3; c) pulse voltage 1400, pulse width: 20 msec, number of pulses: 1; and d) pulse voltage 1300, pulse width: 10 msec, number of pulses: 3.

Particularly suitable conditions for electroporation of erythroid progenitor cells include, e.g., for 8-9 days: a) pulse voltage: 1400-1600, pulse width: 20, number of pulses: 1; b) pulse voltage: 1100-1300, pulse width: 30, number of pulses: 1; c) pulse voltage: 1000-1200, pulse width: 40, number of pulses: 1; d) pulse voltage: 1100-1400, pulse width: 20, number of pulses: 2; e) pulse voltage: 950-1150, pulse width: 30, number of pulses: 2; f) pulse voltage: 1300-1600, pulse width: 10, number of pulses: 3. These conditions generally lead to transfections efficiencies of at least about 60% or more (e.g. at least about 65%, 70%, 75%, 80%, 85%, 90%, 95% or at least about 97%, or more), and cell viability of at least about 70% or more (e.g. at least about 75%, 80%, 85%, 90%, 95% or at least about 97%, or more).

Particularly suitable conditions for electroporation of erythroid progenitor cells in culture under differentiation conditions include, e.g. for 12-13 days: a) pulse voltage: 1500-1700, pulse width: 20, number of pulses: 1; and b) pulse voltage: 1500-1600, pulse width: 10, number of pulses: 3. These conditions generally lead to transfections efficiencies of at least about 50% or more (e.g. at least about 55%, 60%, 65%, 70%, 75%, 80%, 85%, 90%, 95% or at least about 97%, or more), and cell viability of at least about 70% or more (e.g. at least about 75%, 80%, 85%, 90%, 95% or at least about 97%, or more).

The conditions disclosed herein with reference to the Life Technologies Neon system can easily be adjusted by one of ordinary skill in the art to fit a different electroporator and/or different electroporation set-ups with only routine experimentation and the specific electroporator described herein is not limiting for the methods disclosed.

In some embodiments, using the electroporation conditions described herein cultured erythroid progenitor cells are electroporated for a first time, then cultured for a desired period of time (optionally under differentiation conditions) and then re-electroporated a second time. In some embodiments, cultured erythroid progenitor cells are electroporated for a first time, then cultured for a desired period of time (optionally under differentiation conditions) and then re-electroporated a second, third, fourth, fifth, or sixth time. Optionally, the culturing period in between the first and second, the second and third, etc. electroporation can be varied. For example, the period in between electroporations may be adjusted as desired, e.g. the period may be 30 minutes, 1 hour, 6 hours, 12, hours, 18 hours, 24 hours, 30 hours, 36 hours, 48 hours, 3 days, 4 days, 5 days, 6 days, 7 days, 8 days, 9 days, 10 days, 11 days 12 days, 13 days 14 days, or 21 days. For example, erythroid progenitor cells may be electroporated on day 1 and 2, 1 and 3, 1 and 4, 1 and 5, 1 and 6, 1 and 7, 1 and 8, 1 and 9, 1 and 10, 1 and 11, 1 and 12, 1 and 13, 1 and 14, 1 and 15, or 1 and 16. In another example, cells may be electroporated on day 2 and 3, 2 and 4, 2 and 5, 2 and 6, 2 and 7, 2 and 8, 2 and 9, 2 and 10, 2 and 11, 2 and 12, 2 and 13, 2 and 14, 2 and 15, or 2 and 16. In yet another example, erythroid progenitor cells may be electroporated on day 3 and 4, 3 and 5, 3 and 6, 3 and 7, 3 and 8, 3 and 9, 3 and 10, 3 and 11, 3 and 12, 3 and 13, 3 and 14, 3 and 15, or 3 and 16. In yet another example, cells may be electroporated on day 4 and 5, 4 and 6, 4 and 7, 4 and 8, 4 and 9, 4 and 10, 4 and 11, 4 and 12, 4 and 13, 4 and 14, 4 and 15, or 4 and 16. In yet another example, cells may be electroporated on day 5 and 6, 5 and 7, 5 and 8, 5 and 9, 5 and 10, 5 and 11, 5 and 12, 5 and 13, 5 and 14, 5 and 15, or 5 and 16. In yet another example, erythroid progenitor cells may be electroporated on day 6 and 7, 6 and 8, 6 and 9, 6 and 10, 6 and 11, 6 and 12, 6 and 13, 6 and 14, 6 and 15, or 6 and 16. In yet another example, erythroid progenitor cells may be electroporated on day 7 and 8, 7 and 9, 7 and 10, 7 and 11, 7 and 12, 7 and 13, 7 and 14, 7 and 15, or 7 and 16. In yet another example, erythroid progenitor cells may be electroporated on day 8 and 9, 8 and 10, 8 and 11, 8 and 12, 8 and 13, 8 and 14, 8 and 15, or 8 and 16. In yet another example, erythroid progenitor cells may be electroporated on day 9 10, 9 and 11, 9 and 12, 9 and 13, 9 and 14, 9 and 15, or 9 and 16. In yet another example, erythroid progenitor cells may be electroporated on day 10 and 11, 10 and 12, 10 and 13, 10 and 14, 10 and 15, or 10 and 16. In yet another example, erythroid progenitor cells may be electroporated on day 11 and 12, 11 and 13, 11 and 14, 11 and 15, or 11 and 16. In yet another example, erythroid progenitor cells may be electroporated on day 12 and 13, 12 and 14, 12 and 15, or 12 and 16. In yet another example, erythroid progenitor cells may be electroporated on day 13 and 14, 13 and 15, or 13 and 16. In yet another example, erythroid progenitor cells may be electroporated on day 14 and 15, or 14 and 16. Optionally, the erythroid progenitor cells may be electroporated more than twice, e.g., three times, four times, five times, or six times and the interval may be selected as desired at any points of the differentiation process of the cells.

In some embodiments, using the electroporation conditions described herein, cultured erythroid progenitor cells are electroporated on day 1, 2, 3, 4, 5, 6, 7, 8, 9, 10, 11, 12, 13, 14, 15, or 16 of differentiation.

In some embodiments, the engineered enucleated erythroid cells can be click-conjugated engineered enucleated erythroid cells. A catalytic bond-forming polypeptide domain can be expressed on or in, e.g., an erythroid progenitor cell, present in the cytosol or present on the membrane. Many catalytic bond-forming polypeptides exist, including transpeptidases, sortases, and isopeptidases, including those derived from Spy0128, a protein isolated from Streptococcus pyogenes. It has been demonstrated that splitting the autocatalytic isopeptide bond-forming subunit (CnaB2 domain) of Spy0128 results in two distinct polypeptides that retain catalytic activity with specificity for each other. The polypeptides in this system are termed SpyTag and SpyCatcher. Upon mixing, SpyTag and SpyCatcher undergo isopeptide bond formation between Aspl 17 on SpyTag and Lys31 on SpyCatcher (Zakeri and Howarth, JACS 132:4526, 2010). The reaction is compatible with the cellular environment and highly specific for protein/peptide conjugation (Zakeri et al., Proc. Natl. Acad. Sci. U.S.A. 109:E690-E697, 2012). SpyTag and SpyCatcher have been shown to direct post-translational topological modification in elastin-like protein. For example, placement of SpyTag at the N-terminus and SpyCatcher at the C-terminus directs formation of circular elastin-like proteins (Zhang et al, J. Am. Chem. Soc. 2013).

The components SpyTag and SpyCatcher can be interchanged such that a system in which molecule A is fused to SpyTag and molecule B is fused to SpyCatcher is functionally equivalent to a system in which molecule A is fused to SpyCatcher and molecule B is fused to SpyTag. For the purposes of this disclosure, when SpyTag and SpyCatcher are used, it is to be understood that the complementary molecule could be substituted in its place.

A catalytic bond-forming polypeptide, such as a SpyTag/SpyCatcher system, can be used to attach the exogenous protein to the surface of, e.g., an erythroid progenitor cell or an enucleated erythroid cell. The SpyTag polypeptide sequence can be expressed on the extracellular surface of the erytroid progenitor cell or the enucleated erythroid cell. The SpyTag polypeptide can be, for example, fused to the N terminus of a type-1 or type-3 transmembrane protein, e.g., glycophorin A, fused to the C terminus of a type-2 transmembrane protein, e.g., Kell, inserted in-frame at the extracellular terminus or in an extracellular loop of a multi-pass transmembrane protein, e.g., Band 3, fused to a GPI-acceptor polypeptide, e.g., CD55 or CD59, fused to a lipid-chain-anchored polypeptide, or fused to a peripheral membrane protein. An exogenous protein can be fused to SpyCatcher. The nucleic acid encoding the SpyCatcher fusion can be expressed and secreted from the same erythroid progenitor cell or enucleated erythroid cell that expresses the SpyTag fusion. Alternatively, the nucleic acid sequence encoding the SpyCatcher fusion can be produced exogenously, for example in a bacterial, fungal, insect, mammalian, or cell-free production system. Upon reaction of the SpyTag and SpyCatcher polypeptides, a covalent bond will be formed that attaches the exogenous protein to the surface of the erythroid progenitor cell or the enucleated erythroid cell.

In one embodiment, the SpyTag polypeptide may be expressed as a fusion to the N terminus of glycophorin A under the control of the Gatal promoter in an erythroid cell. An exogenous protein fused to the SpyCatcher polypeptide sequence can be expressed under the control of the Gatal promoter in the same erythroid cell. Upon expression of both fusion polypeptides, an isopeptide bond will be formed between the SpyTag and SpyCatcher polypeptides, forming a covalent bond between the erythroid cell surface and the exogenous protein.

In another embodiment, the SpyTag polypeptide may be expressed as a fusion to the N terminus of glycophorin A under the control of the Gatal promoter in an erythroid progenitor cell or an enucleated erythroid cell. An exogenous protein fused to the SpyCatcher polypeptide sequence can be expressed in a suitable mammalian cell expression system, for example HEK293 cells. Upon expression of the SpyTag fusion polypeptide on the erythroid progenitor cell or enucleated erythroid cell, the SpyCatcher fusion polypeptide can be brought in contact with the cell. Under suitable reaction conditions, an isopeptide bond will be formed between the SpyTag and SpyCatcher polypeptides, forming a covalent bond between the erythroid progenitor cell surface or enucleated erythroid cell surface and the exogenous protein.

A catalytic bond-forming polypeptide, such as a SpyTag/SpyCatcher system, can be used to anchor an exogenous protein to the intracellular space of an erythroid progenitor cell or enucleated erythroid cell. The SpyTag polypeptide sequence can be expressed in the intracellular space of the erythroid progenitor cell or enucleated erythroid cell by a number of methods, including direct expression of the transgene, fusion to an endogenous intracellular protein such as, e.g., hemoglobin, fusion to the intracellular domain of endogenous cell surface proteins such as, e.g., Band 3, glycophorin A, Kell, or fusion to a structural component of the cytoskeleton. The SpyTag sequence is not limited to a polypeptide terminus and may be integrated within the interior sequence of an endogenous polypeptide such that polypeptide translation and localization is not perturbed. An exogenous protein can be fused to SpyCatcher. The nucleic acid sequence encoding the SpyCatcher fusion can be expressed within the same erythroid progenitor cell or enucleated erythroid cell that expresses the SpyTag fusion. Upon reaction of the SpyTag and SpyCatcher polypeptides, a covalent bond will be formed that acts to anchor the exogenous protein in the intracellular space of the erythroid progenitor cell or enucleated erythroid cell.

In one embodiment, an erythroid progenitor cell or an enucleated erythroid cell may express SpyTag fused to hemoglobin beta intracellularly. The erythroid progenitor cell or enucleated erythroid cell may be genetically modified with a gene sequence that includes a hemoglobin promoter, beta globin gene, and a SpyTag sequence such that upon translation, intracellular beta globin is fused to SpyTag at is C terminus. In addition, the erythroid progenitor cell or enucleated erythroid cell expresses a Gatal promoter-led gene that codes for SpyCatcher driving protein expression (e.g., phenylalanine hydroxylase (PAH) expression) such that upon translation, intracellular protein (e.g., PAH) is fused to SpyCatcher at its N terminus. Upon expression of both fusion proteins the SpyTag bound beta globin is linked through an isopeptide bond to the SpyCatcher bound protein (e.g., PAH) in the intracellular space, allowing the protein (e.g., PAH) to be anchored to beta globin and retained during maturation.

In another embodiment, the SpyTag polypeptide can be expressed as a fusion to the exogenous protein within an erythroid progenitor cell or an enucleated erythroid cell. The SpyCatcher polypeptide can be expressed as a fusion to the C terminus (intracellular) of glycophorin A within the same erythroid progenitor cell or enucleated erythroid cell. Upon expression of both fusion polypeptides, an isopeptide bond will be formed between the SpyTag and SpyCatcher polypeptides, forming a covalent bond between the membrane-anchored endogenous erythroid polypeptide and the exogenous protein.

Other molecular fusions may be formed between polypeptides and include direct or indirect conjugation. The polypeptides may be directly conjugated to each other or indirectly through a linker. The linker may be a peptide, a polymer, an aptamer, or a nucleic acid. The polymer may be, e.g., natural, synthetic, linear, or branched. Exogenous proteins can comprise a heterologous fusion protein that comprises a first polypeptide and a second polypeptide with the fusion protein comprising the polypeptides directly joined to each other or with intervening linker sequences and/or further sequences at one or both ends. The conjugation to the linker may be through covalent bonds or ionic bonds.

In some embodiments, the engineered enucleated erythroid cells are human enucleated erythroid cells that have been hypotonically loaded. For hypotonic loading/lysis, erythroid progenitor cells or enucleated erythroid cells are exposed to low ionic strength buffer, causing them to burst. The exogenous protein distributes within the cells. Enucleated erythroid cells or erythroid progenitor cells may be hypotonically lysed by adding 30-50 fold volume excess of 5 mM phosphate buffer (pH 8) to a pellet of isolated enucleated erythroid cells. The resulting lysed cell membranes are isolated by centrifugation. The pellet of lysed cell membranes is resuspended and incubated in the presence of the exogenous protein in a low ionic strength buffer, e.g., for 30 min. Alternatively, the lysed cell membranes may be incubated with the exogenous protein for as little as one minute or as long as several days, depending upon the best conditions determined to efficiently load the enucleated erythroid cells or erythroid progenitor cells. For hypotonic loading of a nucleic acid encoding one or more exogenous protein(s) (e.g., any of the exemplary exogenous proteins described herein or known in the art), a nucleic acid can be suspended in a hypotonic Tris-HCl solution (pH 7.0) and injected into erythroid progenitor cells. The concentration of Tris-HCl can be from about 20 mmol/1 to about 150 mmol/1, depending upon the best conditions determined to efficiently load the enucleated erythroid cells.

Alternatively, erythroid progenitor cells or enucleated erythroid cells may be loaded with an exogenous protein using controlled dialysis against a hypotonic solution to swell the cells and create pores in the cell membrane (See, e.g., U.S. Pat. Nos. 4,327,710; 5,753,221; 6,495,351, and 10,046,009). For example, a pellet of cells is resuspended in 10 mM HEPES, 140 mM NaCl, 5 mM glucose pH 7.4 and dialyzed against a low ionic strength buffer containing 10 mM NaH₂P0₄, 10 mM NaHCO₃, 20 mM glucose, and 4 mM MgCl₂, pH 7.4. After 30-60 min, the cells are further dialyzed against 16 mM NaH₂P0₄, pH 7.4 solution containing the exogenous protein for an additional 30-60 min. All of these procedures may be advantageously performed at a temperature of 4° C. In some instances, it may be beneficial to load a large quantity of erythroid progenitor cells or enucleated erythroid cells by a dialysis approach and a specific apparatus designed for this purpose may be used (See, e.g., U.S. Pat. Nos. 4,327,710, 6,139,836 and 6,495,351).

### Buffers

The formulations described herein include a buffer (e.g., one or more buffers) (e.g., any of the exemplary buffers described herein or known in the art).

Non-limiting examples of a buffer (e.g., one or more buffers) that can be present in any of the formulations described herein can be a Good's buffer. Non-limiting examples of Good's buffers include: 4-(2-hydroxyethyl)-1-piperazineethanesulfonic acid (HEPES), 3-(N-morpholino)propanesulfonic acid (MOPS), 2-[[1,3-dihydoxy-2-(hydroxymethyl)propan-2-yl]amino]ethanesulfonic acid (TES), 2-(N-morpholino)ethanesulfonic acid (MES), 2-[(2-amino-2-oxoethyl)-(carboxymethyl)amino]acetic acid (ADA), N-(2-acetamido)-2-aminoethanesulfonic acid (ACES), N,N-bis(2-hydroxyethyl)-2-aminoethanesulfonic acid (BES), 2-(bis(2-hydroxyethyl)amino)acetic acid (Bicine), N-cyclohexyl-3-aminopropanesulfonic acid (CAPS), N-cyclohexyl-2-hydroxyl-3-aminopropanesulfonic acid (CAPSO), N-cyclohexyl-2-aminoethanesulfonic acid (CHES), piperazine-N,N″-bis(2-ethanesulfonic acid) (PIPES), [tris(hydroxymethyl)methylamino]propanesulfonic acid (TAPS), and 2-amino-2-(hydroxymethyl)propane-1,3-diol (Tris). Additional examples of buffers that can be present in any of the formulations described herein are known in the art.

The final concentration of a buffer (or a final total concentration of one or more buffers) in any of the pharmaceutically acceptable aqueous buffered solutions described herein can be, e.g., about 0.1 mM to about 100 mM, about 0.1 mM to about 95 mM, about 0.1 mM to about 90 mM, about 0.1 mM to about 85 mM, about 0.1 mM to about 80 mM, about 0.1 mM to about 75 mM, about 0.1 mM to about 70 mM, about 0.1 mM to about 65 mM, about 0.1 mM to about 60 mM, 0.1 mM to about 55 mM, about 0.1 mM to about 50 mM, about 0.1 mM to about 45 mM, about 0.1 mM to about 40 mM, about 0.1 mM to about 35 mM, about 0.1 mM to about 30 mM, about 0.1 mM to about 25 mM, about 0.1 mM to about 20 mM, about 0.1 mM to about 15 mM, about 0.1 mM to about 10 mM, about 0.1 mM to about 5.0 mM, about 0.1 mM to about 2.0 mM, about 0.1 mM to about 1.0 mM, about 1.0 mM to about 100 mM, about 1.0 mM to about 95 mM, about 1.0 mM to about 90 mM, about 1.0 mM to about 85 mM, about 1.0 mM to about 80 mM, about 1.0 mM to about 75 mM, about 1.0 mM to about 70 mM, about 1.0 mM to about 65 mM, about 1.0 mM to about 60 mM, 1.0 mM to about 55 mM, about 1.0 mM to about 50 mM, about 1.0 mM to about 45 mM, about 1.0 mM to about 40 mM, about 1.0 mM to about 35 mM, about 1.0 mM to about 30 mM, about 1.0 mM to about 25 mM, about 1.0 mM to about 20 mM, about 1.0 mM to about 15 mM, about 1.0 mM to about 10 mM, about 1.0 mM to about 5.0 mM, about 1.0 mM to about 2.0 mM, about 5.0 mM to about 100 mM, about 5.0 mM to about 95 mM, about 5.0 mM to about 90 mM, about 5.0 mM to about 85 mM, about 5.0 mM to about 80 mM, about 5.0 mM to about 75 mM, about 5.0 mM to about 70 mM, about 5.0 mM to about 65 mM, about 5.0 mM to about 60 mM, about 5.0 mM to about 55 mM, about 5.0 mM to about 50 mM, about 5.0 mM to about 45 mM, about 5.0 mM to about 40 mM, about 5.0 mM to about 35 mM, about 5.0 mM to about 30 mM, about 5.0 mM to about 25 mM, about 5.0 mM to about 20 mM, about 5.0 mM to about 15 mM, about 5.0 mM to about 10 mM, about 10 mM to about 100 mM, about 10 mM to about 95 mM, about 10 mM to about 90 mM, about 10 mM to about 85 mM, about 10 mM to about 80 mM, about 10 mM to about 75 mM, about 10 mM to about 70 mM, about 10 mM to about 65 mM, about 10 mM to about 60 mM, about 10 mM to about 55 mM, about 10 mM to about 50 mM, about 10 mM to about 45 mM, about 10 mM to about 40 mM, about 10 mM to about 35 mM, about 10 mM to about 30 mM, about 10 mM to about 25 mM, about 10 mM to about 20 mM, about 10 mM to about 15 mM, about 15 mM to about 100 mM, about 15 mM to about 95 mM, about 15 mM to about 90 mM, about 15 mM to about 85 mM, about 15 mM to about 80 mM, about 15 mM to about 75 mM, about 15 mM to about 70 mM, about 15 mM to about 65 mM, about 15 mM to about 60 mM, about 15 mM to about 55 mM, about 15 mM to about 50 mM, about 15 mM to about 45 mM, about 15 mM to about 40 mM, about 15 mM to about 35 mM, about 15 mM to about 30 mM, about 15 mM to about 25 mM, about 15 mM to about 20 mM, about 20 mM to about 100 mM, about 20 mM to about 95 mM, about 20 mM to about 90 mM, about 20 mM to about 85 mM, about 20 mM to about 80 mM, about 20 mM to about 75 mM, about 20 mM to about 70 mM, about 20 mM to about 65 mM, about 20 mM to about 60 mM, about 20 mM to about 55 mM, about 20 mM to about 50 mM, about 20 mM to about 45 mM, about 20 mM to about 40 mM, about 20 mM to about 35 mM, about 20 mM to about 30 mM, or about 20 mM to about 25 mM.

### Phosphate Ion

The final concentration of a phosphate ion in any of the pharmaceutically acceptable aqueous buffered solutions described herein can be, e.g., about 0.1 mM to about 50 mM, about 0.1 mM to about 45 mM, about 0.1 mM to about 40 mM, about 0.1 mM to about 35 mM, about 0.1 mM to about 30 mM, about 0.1 mM to about 25 mM, about 0.1 mM to about 20 mM, about 0.1 mM to about 15 mM, about 0.1 mM to about 10 mM, about 0.1 mM to about 5.0 mM, about 0.1 mM to about 2.0 mM, about 0.1 mM to about 1.0 mM, about 1.0 mM to about 50 mM, about 1.0 mM to about 45 mM, about 1.0 mM to about 40 mM, about 1.0 mM to about 35 mM, about 1.0 mM to about 30 mM, about 1.0 mM to about 25 mM, about 1.0 mM to about 20 mM, about 1.0 mM to about 15 mM, about 1.0 mM to about 10 mM, about 1.0 mM to about 5.0 mM, about 1.0 mM to about 2.0 mM, about 5.0 mM to about 50 mM, about 5.0 mM to about 45 mM, about 5.0 mM to about 40 mM, about 5.0 mM to about 35 mM, about 5.0 mM to about 30 mM, about 5.0 mM to about 25 mM, about 5.0 mM to about 20 mM, about 5.0 mM to about 15 mM, about 5.0 mM to about 10 mM, about 10 mM to about 50 mM, about 10 mM to about 45 mM, about 10 mM to about 40 mM, about 10 mM to about 35 mM, about 10 mM to about 30 mM, about 10 mM to about 25 mM, about 10 mM to about 20 mM, about 10 mM to about 15 mM, about 15 mM to about 50 mM, about 15 mM to about 45 mM, about 15 mM to about 40 mM, about 15 mM to about 35 mM, about 15 mM to about 30 mM, about 15 mM to about 25 mM, about 15 mM to about 20 mM, about 20 mM to about 50 mM, about 20 mM to about 45 mM, about 20 mM to about 40 mM, about 20 mM to about 35 mM, about 20 mM to about 30 mM, about 20 mM to about 25 mM, about 25 mM to about 50 mM, about 25 mM to about 45 mM, about 25 mM to about 40 mM, about 25 mM to about 35 mM, about 25 mM to about 30 mM, about 30 mM to about 50 mM, about 30 mM to about 45 mM, about 30 mM to about 40 mM, about 30 mM to about 35 mM, about 35 mM to about 50 mM, about 35 mM to about 45 mM, about 35 mM to about 40 mM, about 40 mM to about 50 mM, about 40 mM to about 45 mM, or about 45 mM to about 50 mM.

In some embodiments, the phosphate ion is present in the pharmaceutically acceptable aqueous buffered solution as monosodium phosphate, disodium phosphate, monocalcium phosphate, dicalcium phosphate, pentapotassium triphosphate, pentasodium triphosphate, magnesium phosphate, potassium phosphate, or ammonium phosphate. Additional pharmaceutically acceptable sources of phosphate ion are known in the art.

### Sodium Ion

The final concentration of a sodium ion in any of the pharmaceutically acceptable aqueous buffered solutions described herein can be, e.g., about 20 mM to about 200 mM, about 20 mM to about 190 mM, about 20 mM to about 180 mM, about 20 mM to about 170 mM, about 20 mM to about 160 mM, about 20 mM to about 150 mM, about 20 mM to about 140 mM, about 20 mM to about 130 mM, about 20 mM to about 120 mM, about 20 mM to about 110 mM, about 20 mM to about 100 mM, about 20 mM to about 90 mM, about 20 mM to about 80 mM, about 20 mM to about 70 mM, about 20 mM to about 60 mM, about 20 mM to about 50 mM, about 20 mM to about 40 mM, about 20 mM to about 30 mM, about 30 mM to about 200 mM, about 30 mM to about 190 mM, about 30 mM to about 180 mM, about 30 mM to about 170 mM, about 30 mM to about 160 mM, about 30 mM to about 150 mM, about 30 mM to about 140 mM, about 30 mM to about 130 mM, about 30 mM to about 120 mM, about 30 mM to about 110 mM, about 30 mM to about 100 mM, about 30 mM to about 90 mM, about 30 mM to about 80 mM, about 30 mM to about 70 mM, about 30 mM to about 60 mM, about 30 mM to about 50 mM, about 30 mM to about 40 mM, about 40 mM to about 200 mM, about 40 mM to about 190 mM, about 40 mM to about 180 mM, about 40 mM to about 170 mM, about 40 mM to about 160 mM, about 40 mM to about 150 mM, about 40 mM to about 140 mM, about 40 mM to about 130 mM, about 40 mM to about 120 mM, about 40 mM to about 110 mM, about 40 mM to about 100 mM, about 40 mM to about 90 mM, about 40 mM to about 80 mM, about 40 mM to about 70 mM, about 40 mM to about 60 mM, about 40 mM to about 50 mM, about 50 mM to about 200 mM, about 50 mM to about 190 mM, about 50 mM to about 180 mM, about 50 mM to about 170 mM, about 50 mM to about 160 mM, about 50 mM to about 150 mM, about 50 mM to about 140 mM, about 50 mM to about 130 mM, about 50 mM to about 120 mM, about 50 mM to about 110 mM, about 50 mM to about 100 mM, about 50 mM to about 90 mM, about 50 mM to about 80 mM, about 50 mM to about 70 mM, about 50 mM to about 60 mM, about 60 mM to about 200 mM, about 60 mM to about 190 mM, about 60 mM to about 180 mM, about 60 mM to about 170 mM, about 60 mM to about 160 mM, about 60 mM to about 150 mM, about 60 mM to about 140 mM, about 60 mM to about 130 mM, about 60 mM to about 120 mM, about 60 mM to about 110 mM, about 60 mM to about 100 mM, about 60 mM to about 90 mM, about 60 mM to about 80 mM, about 60 mM to about 70 mM, about 70 mM to about 200 mM, about 70 mM to about 190 mM, about 70 mM to about 180 mM, about 70 mM to about 170 mM, about 70 mM to about 160 mM, about 70 mM to about 150 mM, about 70 mM to about 140 mM, about 70 mM to about 130 mM, about 70 mM to about 120 mM, about 70 mM to about 110 mM, about 70 mM to about 100 mM, about 70 mM to about 90 mM, about 70 mM to about 80 mM, about 80 mM to about 200 mM, about 80 mM to about 190 mM, about 80 mM to about 180 mM, about 80 mM to about 170 mM, about 80 mM to about 160 mM, about 80 mM to about 150 mM, about 80 mM to about 140 mM, about 80 mM to about 130 mM, about 80 mM to about 120 mM, about 80 mM to about 110 mM, about 80 mM to about 100 mM, about 80 mM to about 90 mM, about 90 mM to about 200 mM, about 90 mM to about 190 mM, about 90 mM to about 180 mM, about 90 mM to about 170 mM, about 90 mM to about 160 mM, about 90 mM to about 150 mM, about 90 mM to about 140 mM, about 90 mM to about 130 mM, about 90 mM to about 120 mM, about 90 mM to about 110 mM, about 90 mM to about 100 mM, about 100 mM to about 200 mM, about 100 mM to about 190 mM, about 100 mM to about 180 mM, about 100 mM to about 170 mM, about 100 mM to about 160 mM, about 100 mM to about 150 mM, about 100 mM to about 140 mM, about 100 mM to about 130 mM, about 100 mM to about 120 mM, about 100 mM to about 110 mM, about 110 mM to about 200 mM, about 110 mM to about 190 mM, about 110 mM to about 180 mM, about 110 mM to about 170 mM, about 110 mM to about 160 mM, about 110 mM to about 150 mM, about 110 mM to about 140 mM, about 110 mM to about 130 mM, about 110 mM to about 120 mM, about 120 mM to about 200 mM, about 120 mM to about 190 mM, about 120 mM to about 180 mM, about 120 mM to about 170 mM, about 120 mM to about 160 mM, about 120 mM to about 150 mM, about 120 mM to about 140 mM, about 120 mM to about 130 mM, about 130 mM to about 200 mM, about 130 mM to about 190 mM, about 130 mM to about 180 mM, about 130 mM to about 170 mM, about 130 mM to about 160 mM, about 130 mM to about 150 mM, about 130 mM to about 140 mM, about 140 mM to about 200 mM, about 140 mM to about 190 mM, about 140 mM to about 180 mM, about 140 mM to about 170 mM, about 140 mM to about 160 mM, about 140 mM to about 150 mM, about 150 mM to about 200 mM, about 150 mM to about 190 mM, about 150 mM to about 180 mM, about 150 mM to about 170 mM, about 150 mM to about 160 mM, about 160 mM to about 200 mM, about 160 mM to about 190 mM, about 160 mM to about 180 mM, about 160 mM to about 170 mM, about 170 mM to about 200 mM, about 170 mM to about 190 mM, about 170 mM to about 180 mM, about 180 mM to about 200 mM, about 180 mM to about 190 mM, or about 190 mM to about 200 mM.

In some embodiments, the sodium ion is present in the pharmaceutically acceptable aqueous buffered solutions described herein as sodium chloride, monosodium phosphate, disodium phosphate, sodium fluoride, sodium bromide, sodium iodide, sodium sulfate, sodium bicarbonate, sodium carbonate, or sodium amide. Additional pharmaceutical acceptable sources of sodium ion are known in the art. In some embodiments, the sodium ion can be provided as the counterion for one or more anions that are present in the composition.

### Potassium Ion

The final concentration of a potassium ion in any of the pharmaceutically acceptable aqueous buffered solutions described herein can be, e.g., about 0.1 mM to about 100 mM, about 0.1 mM to about 95 mM, about 0.1 mM to about 90 mM, about 0.1 mM to about 85 mM, about 0.1 mM to about 80 mM, about 0.1 mM to about 75 mM, about 0.1 mM to about 70 mM, about 0.1 mM to about 65 mM, about 0.1 mM to about 60 mM, about 0.1 mM to about 55 mM, about 0.1 mM to about 50 mM, about 0.1 mM to about 45 mM, about 0.1 mM to about 40 mM, about 0.1 mM to about 35 mM, about 0.1 mM to about 30 mM, about 0.1 mM to about 25 mM, about 0.1 mM to about 20 mM, about 0.1 mM to about 15 mM, about 0.1 mM to about 10 mM, about 0.1 mM to about 5 mM, about 5 mM to about 100 mM, about 5 mM to about 95 mM, about 5 mM to about 90 mM, about 5 mM to about 85 mM, about 5 mM to about 80 mM, about 5 mM to about 75 mM, about 5 mM to about 70 mM, about 5 mM to about 65 mM, about 5 mM to about 60 mM, about 5 mM to about 55 mM, about 5 mM to about 50 mM, about 5 mM to about 45 mM, about 5 mM to about 40 mM, about 5 mM to about 35 mM, about 5 mM to about 30 mM, about 5 mM to about 25 mM, about 5 mM to about 20 mM, about 5 mM to about 15 mM, about 5 mM to about 10 mM, about 10 mM to about 100 mM, about 10 mM to about 95 mM, about 10 mM to about 90 mM, about 10 mM to about 85 mM, about 10 mM to about 80 mM, about 10 mM to about 75 mM, about 10 mM to about 70 mM, about 10 mM to about 65 mM, about 10 mM to about 60 mM, about 10 mM to about 55 mM, about 10 mM to about 50 mM, about 10 mM to about 45 mM, about 10 mM to about 40 mM, about 10 mM to about 35 mM, about 10 mM to about 30 mM, about 10 mM to about 25 mM, about 10 mM to about 20 mM, about 10 mM to about 15 mM, about 15 mM to about 100 mM, about 15 mM to about 95 mM, about 15 mM to about 90 mM, about 15 mM to about 85 mM, about 15 mM to about 80 mM, about 15 mM to about 75 mM, about 15 mM to about 70 mM, about 15 mM to about 65 mM, about 15 mM to about 60 mM, about 15 mM to about 55 mM, about 15 mM to about 50 mM, about 15 mM to about 45 mM, about 15 mM to about 40 mM, about 15 mM to about 35 mM, about 15 mM to about 30 mM, about 15 mM to about 25 mM, about 15 mM to about 20 mM, about 20 mM to about 100 mM, about 20 mM to about 95 mM, about 20 mM to about 90 mM, about 20 mM to about 85 mM, about 20 mM to about 80 mM, about 20 mM to about 75 mM, about 20 mM to about 70 mM, about 20 mM to about 65 mM, about 20 mM to about 60 mM, about 20 mM to about 55 mM, about 20 mM to about 50 mM, about 20 mM to about 45 mM, about 20 mM to about 40 mM, about 20 mM to about 35 mM, about 20 mM to about 30 mM, about 20 mM to about 25 mM, about 30 mM to about 100 mM, about 30 mM to about 95 mM, about 30 mM to about 90 mM, about 30 mM to about 85 mM, about 30 mM to about 80 mM, about 30 mM to about 75 mM, about 30 mM to about 70 mM, about 30 mM to about 65 mM, about 30 mM to about 60 mM, about 30 mM to about 55 mM, about 30 mM to about 50 mM, about 30 mM to about 45 mM, about 30 mM to about 40 mM, about 30 mM to about 35 mM, about 40 mM to about 100 mM, about 40 mM to about 95 mM, about 40 mM to about 90 mM, about 40 mM to about 85 mM, about 40 mM to about 80 mM, about 40 mM to about 75 mM, about 40 mM to about 70 mM, about 40 mM to about 65 mM, about 40 mM to about 60 mM, about 40 mM to about 55 mM, about 40 mM to about 50 mM, about 40 mM to about 45 mM, about 50 mM to about 100 mM, about 50 mM to about 95 mM, about 50 mM to about 90 mM, about 50 mM to about 85 mM, about 50 mM to about 80 mM, about 50 mM to about 75 mM, about 50 mM to about 70 mM, about 50 mM to about 65 mM, about 50 mM to about 60 mM, about 50 mM to about 55 mM, about 60 mM to about 100 mM, about 60 mM to about 95 mM, about 60 mM to about 90 mM, about 60 mM to about 85 mM, about 60 mM to about 80 mM, about 60 mM to about 75 mM, about 60 mM to about 70 mM, about 60 mM to about 65 mM, about 70 mM to about 100 mM, about 70 mM to about 95 mM, about 70 mM to about 90 mM, about 70 mM to about 85 mM, about 70 mM to about 80 mM, about 70 mM to about 75 mM, about 80 mM to about 100 mM, about 80 mM to about 95 mM, about 80 mM to about 90 mM, about 80 mM to about 85 mM, about 90 mM to about 100 mM, or about 90 mM to about 95 mM.

In some embodiments, the potassium ion is present in the pharmaceutically acceptable solutions described herein as potassium chloride, potassium bisulfate, potassium carbonate, potassium fluoride, potassium idodide, potassium nitrate, potassium phosphate, or potassium sulfate. Additional pharmaceutically acceptable sources of potassium ion are known in the art.

### Calcium Ion

The final concentration of a calcium ion in any of the pharmaceutically acceptable aqueous buffered solutions described herein can be, e.g., about 0.01 mM to about 20 mM, about 0.01 mM to about 19 mM, about 0.01 mM to about 18 mM, about 0.01 mM to about 17 mM, about 0.01 mM to about 16 mM, about 0.01 mM to about 15 mM, about 0.01 mM to about 14 mM, about 0.01 mM to about 13 mM, about 0.01 mM to about 12 mM, about 0.01 mM to about 11 mM, about 0.01 mM to about 10 mM, about 0.01 mM to about 9 mM, about 0.01 mM to about 8 mM, about 0.01 mM to about 7 mM, about 0.01 mM to about 6 mM, about 0.01 mM to about 5 mM, about 0.01 mM to about 4 mM, about 0.01 mM to about 3 mM, about 0.01 mM to about 2 mM, about 0.01 mM to about 1 mM, about 0.01 mM to about 0.1 mM, about 0.01 mM to about 0.05 mM, about 0.05 mM to about 20 mM, about 0.05 mM to about 19 mM, about 0.05 mM to about 18 mM, about 0.05 mM to about 17 mM, about 0.05 mM to about 16 mM, about 0.05 mM to about 15 mM, about 0.05 mM to about 14 mM, about 0.05 mM to about 13 mM, about 0.05 mM to about 12 mM, about 0.05 mM to about 11 mM, about 0.05 mM to about 10 mM, about 0.05 mM to about 9 mM, about 0.05 mM to about 8 mM, about 0.05 mM to about 7 mM, about 0.05 mM to about 6 mM, about 0.05 mM to about 5 mM, about 0.05 mM to about 4 mM, about 0.05 mM to about 3 mM, about 0.05 mM to about 2 mM, about 0.05 mM to about 1 mM, about 0.05 mM to about 0.5 mM, about 0.05 mM to about 0.1 mM, about 0.1 mM to about 20 mM, about 0.1 mM to about 19 mM, about 0.1 mM to about 18 mM, about 0.1 mM to about 17 mM, about 0.1 mM to about 16 mM, about 0.1 mM to about 15 mM, about 0.1 mM to about 14 mM, about 0.1 mM to about 13 mM, about 0.1 mM to about 12 mM, about 0.1 mM to about 11 mM, about 0.1 mM to about 10 mM, about 0.1 mM to about 9 mM, about 0.1 mM to about 8 mM, about 0.1 mM to about 7 mM, about 0.1 mM to about 6 mM, about 0.1 mM to about 5 mM, about 0.1 mM to about 4 mM, about 0.1 mM to about 3 mM, about 0.1 mM to about 2 mM, about 0.1 mM to about 1 mM, about 0.1 mM to about 0.5 mM, about 0.5 mM to about 20 mM, about 0.5 mM to about 19 mM, about 0.5 mM to about 18 mM, about 0.5 mM to about 17 mM, about 0.5 mM to about 16 mM, about 0.5 mM to about 15 mM, about 0.5 mM to about 14 mM, about 0.5 mM to about 13 mM, about 0.5 mM to about 12 mM, about 0.5 mM to about 11 mM, about 0.5 mM to about 10 mM, about 0.5 mM to about 9 mM, about 0.5 mM to about 8 mM, about 0.5 mM to about 7 mM, about 0.5 mM to about 6 mM, about 0.5 mM to about 5 mM, about 0.5 mM to about 4 mM, about 0.5 mM to about 3 mM, about 0.5 mM to about 2 mM, about 0.5 mM to about 1.0 mM, about 1 mM to about 20 mM, about 1 mM to about 19 mM, about 1 mM to about 18 mM, about 1 mM to about 17 mM, about 1 mM to about 16 mM, about 1 mM to about 15 mM, about 1 mM to about 14 mM, about 1 mM to about 13 mM, about 1 mM to about 12 mM, about 1 mM to about 11 mM, about 1 mM to about 10 mM, about 1 mM to about 9 mM, about 1 mM to about 8 mM, about 1 mM to about 7 mM, about 1 mM to about 6 mM, about 1 mM to about 5 mM, about 1 mM to about 4 mM, about 1 mM to about 3 mM, about 1 mM to about 2 mM, about 2 mM to about 20 mM, about 2 mM to about 19 mM, about 2 mM to about 18 mM, about 2 mM to about 17 mM, about 2 mM to about 16 mM, about 2 mM to about 15 mM, about 2 mM to about 14 mM, about 2 mM to about 13 mM, about 2 mM to about 12 mM, about 2 mM to about 11 mM, about 2 mM to about 10 mM, about 2 mM to about 9 mM, about 2 mM to about 8 mM, about 2 mM to about 7 mM, about 2 mM to about 6 mM, about 2 mM to about 5 mM, about 2 mM to about 4 mM, about 2 mM to about 3 mM, about 3 mM to about 20 mM, about 3 mM to about 19 mM, about 3 mM to about 18 mM, about 3 mM to about 17 mM, about 3 mM to about 16 mM, about 3 mM to about 15 mM, about 3 mM to about 14 mM, about 3 mM to about 13 mM, about 3 mM to about 12 mM, about 3 mM to about 11 mM, about 3 mM to about 10 mM, about 3 mM to about 9 mM, about 3 mM to about 8 mM, about 3 mM to about 7 mM, about 3 mM to about 6 mM, about 3 mM to about 5 mM, about 3 mM to about 4 mM, about 4 mM to about 20 mM, about 4 mM to about 19 mM, about 4 mM to about 18 mM, about 4 mM to about 17 mM, about 4 mM to about 16 mM, about 4 mM to about 15 mM, about 4 mM to about 14 mM, about 4 mM to about 13 mM, about 4 mM to about 12 mM, about 4 mM to about 11 mM, about 4 mM to about 10 mM, about 4 mM to about 9 mM, about 4 mM to about 8 mM, about 4 mM to about 7 mM, about 4 mM to about 6 mM, about 4 mM to about 5 mM, about 5 mM to about 20 mM, about 5 mM to about 19 mM, about 5 mM to about 18 mM, about 5 mM to about 17 mM, about 5 mM to about 16 mM, about 5 mM to about 15 mM, about 5 mM to about 14 mM, about 5 mM to about 13 mM, about 5 mM to about 12 mM, about 5 mM to about 11 mM, about 5 mM to about 10 mM, about 5 mM to about 9 mM, about 5 mM to about 8 mM, about 5 mM to about 7 mM, about 5 mM to about 6 mM, about 6 mM to about 20 mM, about 6 mM to about 19 mM, about 6 mM to about 18 mM, about 6 mM to about 17 mM, about 6 mM to about 16 mM, about 6 mM to about 15 mM, about 6 mM to about 14 mM, about 6 mM to about 13 mM, about 6 mM to about 12 mM, about 6 mM to about 11 mM, about 6 mM to about 10 mM, about 6 mM to about 9 mM, about 6 mM to about 8 mM, about 6 mM to about 7 mM, about 7 mM to about 20 mM, about 7 mM to about 19 mM, about 7 mM to about 18 mM, about 7 mM to about 17 mM, about 7 mM to about 16 mM, about 7 mM to about 15 mM, about 7 mM to about 14 mM, about 7 mM to about 13 mM, about 7 mM to about 12 mM, about 7 mM to about 11 mM, about 7 mM to about 10 mM, about 7 mM to about 9 mM, about 7 mM to about 8 mM, about 8 mM to about 20 mM, about 8 mM to about 19 mM, about 8 mM to about 18 mM, about 8 mM to about 18 mM, about 8 mM to about 16 mM, about 8 mM to about 15 mM, about 8 mM to about 14 mM, about 8 mM to about 13 mM, about 8 mM to about 12 mM, about 8 mM to about 11 mM, about 8 mM to about 10 mM, about 8 mM to about 9 mM, about 9 mM to about 20 mM, about 9 mM to about 19 mM, about 9 mM to about 19 mM, about 9 mM to about 19 mM, about 9 mM to about 16 mM, about 9 mM to about 15 mM, about 9 mM to about 14 mM, about 9 mM to about 13 mM, about 9 mM to about 12 mM, about 9 mM to about 11 mM, about 9 mM to about 10 mM, about 10 mM to about 20 mM, about 10 mM to about 19 mM, about 10 mM to about 18 mM, about 10 mM to about 17 mM, about 10 mM to about 16 mM, about 10 mM to about 15 mM, about 10 mM to about 14 mM, about 10 mM to about 13 mM, about 10 mM to about 12 mM, about 10 mM to about 11 mM, about 11 mM to about 20 mM, about 11 mM to about 19 mM, about 11 mM to about 18 mM, about 11 mM to about 17 mM, about 11 mM to about 16 mM, about 11 mM to about 15 mM, about 11 mM to about 14 mM, about 11 mM to about 13 mM, about 11 mM to about 12 mM, about 12 mM to about 20 mM, about 12 mM to about 19 mM, about 12 mM to about 18 mM, about 12 mM to about 17 mM, about 12 mM to about 16 mM, about 12 mM to about 15 mM, about 12 mM to about 14 mM, about 12 mM to about 13 mM, about 13 mM to about 20 mM, about 13 mM to about 19 mM, about 13 mM to about 18 mM, about 13 mM to about 17 mM, about 13 mM to about 16 mM, about 13 mM to about 15 mM, about 13 mM to about 14 mM, about 14 mM to about 20 mM, about 14 mM to about 19 mM, about 14 mM to about 18 mM, about 14 mM to about 17 mM, about 14 mM to about 16 mM, about 14 mM to about 15 mM, about 15 mM to about 20 mM, about 15 mM to about 19 mM, about 15 mM to about 18 mM, about 15 mM to about 17 mM, about 15 mM to about 16 mM, about 16 mM to about 20 mM, about 16 mM to about 19 mM, about 16 mM to about 18 mM, about 16 mM to about 17 mM, about 17 mM to about 20 mM, about 17 mM to about 19 mM, about 17 mM to about 18 mM, about 18 mM to about 20 mM, about 18 mM to about 19 mM, or about 19 mM to about 20 mM.

In some embodiments, the calcium ion is present in the pharmaceutically acceptable solutions described herein as calcium chloride, calcium carbonate, calcium iodide, calcium sulfate, calcium phosphate, or calcium nitrite. Additional pharmaceutically acceptable sources of calcium ion are known in the art.

### Magnesium Ion

The final concentration of a magnesium ion in any of the pharmaceutically acceptable aqueous buffered solutions described herein can be, e.g., about 0.1 mM to about 50 mM, about 0.1 mM to about 45 mM, about 0.1 mM to about 40 mM, about 0.1 mM to about 35 mM, about 0.1 mM to about 30 mM, about 0.1 mM to about 25 mM, about 0.1 mM to about 20 mM, about 0.1 mM to about 15 mM, about 0.1 mM to about 10 mM, about 0.1 mM to about 5 mM, about 0.1 mM to about 1 mM, about 0.1 mM to about 0.5 mM, about 0.5 mM to about 50 mM, about 0.5 mM to about 45 mM, about 0.5 mM to about 40 mM, about 0.5 mM to about 35 mM, about 0.5 mM to about 30 mM, about 0.5 mM to about 25 mM, about 0.5 mM to about 20 mM, about 0.5 mM to about 15 mM, about 0.5 mM to about 10 mM, about 0.5 mM to about 5 mM, about 0.5 mM to about 1 mM, about 1 mM to about 50 mM, about 1 mM to about 45 mM, about 1 mM to about 40 mM, about 1 mM to about 35 mM, about 1 mM to about 30 mM, about 1 mM to about 25 mM, about 1 mM to about 20 mM, about 1 mM to about 15 mM, about 1 mM to about 10 mM, about 1 mM to about 5 mM, about 5 mM to about 50 mM, about 5 mM to about 45 mM, about 5 mM to about 40 mM, about 5 mM to about 35 mM, about 5 mM to about 30 mM, about 5 mM to about 25 mM, about 5 mM to about 20 mM, about 5 mM to about 15 mM, about 5 mM to about 10 mM, about 10 mM to about 50 mM, about 10 mM to about 45 mM, about 10 mM to about 40 mM, about 10 mM to about 35 mM, about 10 mM to about 30 mM, about 10 mM to about 25 mM, about 10 mM to about 20 mM, about 10 mM to about 15 mM, about 15 mM to about 50 mM, about 15 mM to about 45 mM, about 15 mM to about 40 mM, about 15 mM to about 35 mM, about 15 mM to about 30 mM, about 15 mM to about 25 mM, about 15 mM to about 20 mM, about 20 mM to about 50 mM, about 20 mM to about 45 mM, about 20 mM to about 40 mM, about 20 mM to about 35 mM, about 20 mM to about 30 mM, about 20 mM to about 25 mM, about 25 mM to about 50 mM, about 25 mM to about 45 mM, about 25 mM to about 40 mM, about 25 mM to about 35 mM, about 25 mM to about 30 mM, about 30 mM to about 50 mM, about 30 mM to about 45 mM, about 30 mM to about 40 mM, about 30 mM to about 35 mM, about 35 mM to about 50 mM, about 35 mM to about 45 mM, about 35 mM to about 40 mM, about 40 mM to about 50 mM, about 40 mM to about 45 mM, or about 45 mM to about 50 mM.

In some embodiments, the magnesium ion is present in the pharmaceutically acceptable solutions described herein as magnesium chloride, magnesium bromide, magnesium fluoride, magnesium iodide, or magnesium sulfate. Additional examples of pharmaceutically acceptable sources of magnesium ion are known in the art.

### Non-Ionic Cell Impermeant Agents

The final concentration of a non-ionic cell impermeant agent in any of the pharmaceutically acceptable aqueous buffered solutions described herein can be, e.g., about 5 mM to about 100 mM, about 5 mM to about 90 mM, about 5 mM to about 80 mM, about 5 mM to about 70 mM, about 5 mM to about 60 mM, about 5 mM to about 50 mM, about 5 mM to about 40 mM, about 5 mM to about 30 mM, about 5 mM to about 20 mM, about 5 mM to about 10 mM, about 10 mM to about 100 mM, about 10 mM to about 90 mM, about 10 mM to about 80 mM, about 10 mM to about 70 mM, about 10 mM to about 60 mM, about 10 mM to about 50 mM, about 10 mM to about 40 mM, about 10 mM to about 30 mM, about 10 mM to about 20 mM, about 20 mM to about 100 mM, about 20 mM to about 90 mM, about 20 mM to about 80 mM, about 20 mM to about 70 mM, about 20 mM to about 60 mM, about 20 mM to about 50 mM, about 20 mM to about 40 mM, about 20 mM to about 30 mM, about 30 mM to about 100 mM, about 30 mM to about 90 mM, about 30 mM to about 80 mM, about 30 mM to about 70 mM, about 30 mM to about 60 mM, about 30 mM to about 50 mM, about 30 mM to about 40 mM, about 40 mM to about 100 mM, about 40 mM to about 90 mM, about 40 mM to about 80 mM, about 40 mM to about 70 mM, about 40 mM to about 60 mM, about 40 mM to about 50 mM, about 50 mM to about 100 mM, about 50 mM to about 90 mM, about 50 mM to about 80 mM, about 50 mM to about 70 mM, about 50 mM to about 60 mM, about 60 mM to about 100 mM, about 60 mM to about 90 mM, about 60 mM to about 80 mM, about 60 mM to about 70 mM, about 70 mM to about 100 mM, about 70 mM to about 90 mM, about 70 mM to about 80 mM, about 80 mM to about 100 mM, about 80 mM to about 90 mM, or about 90 mM to about 100 mM.

Non-limiting examples of non-ionic cell impermeant agents include mannitol, raffinose, sucrose, sorbitol, trehalose, gluconate, and a PEG (e.g., a PEG having a molecular weight of greater than 1 kDa, greater than 5 kDa, or greater than 15 kDa, e.g., PEG 20 kDa). Additional examples of non-ionic cell impermeant agents are known in the art.

### Glucose

In some embodiments, the pharmaceutically acceptable aqueous buffered solutions described herein includes less than 0.1 mM glucose, e.g. less than 0.09 mM, less than 0.08 mM, less than 0.07 mM, less than 0.06 mM, less than 0.05 mM, less than 0.04 mM, less than 0.03 mM, less than 0.02 mM, less than 0.01 mM, less than 0.009 mM, less than 0.008 mM, less than 0.007 mM, less than 0.006 mM, less than 0.005 mM, less than 0.004 mM, less than 0.003 mM, less than 0.002 mM, or less than 0.001 mM. In some embodiments, the pharmaceutically acceptable aqueous buffered solutions described herein includes no glucose. In some embodiments, the pharmaceutically acceptable aqueous buffered solutions described herein includes no detectable glucose.

### Anionic Cell Impermeant Agent

In some embodiments, the pharmaceutically acceptable aqueous buffered solutions described herein can further include an anionic cell impermeant agent. The final concentration of an anionic cell impermeant agent in any of the pharmaceutically acceptable aqueous buffered solutions described herein can be, e.g., about 1 mM to about 150 mM, about 1 mM to about 140 mM, about 1 mM to about 130 mM, about 1 mM to about 120 mM, about 1 mM to about 110 mM, about 1 mM to about 100 mM, about 1 mM to about 90 mM, about 1 mM to about 80 mM, about 1 mM to about 70 mM, about 1 mM to about 60 mM, about 1 mM to about 50 mM, about 1 mM to about 40 mM, about 1 mM to about 30 mM, about 1 mM to about 20 mM, about 1 mM to about 10 mM, about 1 mM to about 5 mM, about 5 mM to about 150 mM, about 5 mM to about 140 mM, about 5 mM to about 130 mM, about 5 mM to about 120 mM, about 5 mM to about 110 mM, about 5 mM to about 100 mM, about 5 mM to about 90 mM, about 5 mM to about 80 mM, about 5 mM to about 70 mM, about 5 mM to about 60 mM, about 5 mM to about 50 mM, about 5 mM to about 40 mM, about 5 mM to about 30 mM, about 5 mM to about 20 mM, about 5 mM to about 10 mM, about 10 mM to about 150 mM, about 10 mM to about 140 mM, about 10 mM to about 130 mM, about 10 mM to about 120 mM, about 10 mM to about 110 mM, about 10 mM to about 100 mM, about 10 mM to about 90 mM, about 10 mM to about 80 mM, about 10 mM to about 70 mM, about 10 mM to about 60 mM, about 10 mM to about 50 mM, about 10 mM to about 40 mM, about 10 mM to about 30 mM, about 10 mM to about 20 mM, about 20 mM to about 150 mM, about 20 mM to about 140 mM, about 20 mM to about 130 mM, about 20 mM to about 120 mM, about 20 mM to about 110 mM, about 20 mM to about 100 mM, about 20 mM to about 90 mM, about 20 mM to about 80 mM, about 20 mM to about 70 mM, about 20 mM to about 60 mM, about 20 mM to about 50 mM, about 20 mM to about 40 mM, about 20 mM to about 30 mM, about 30 mM to about 150 mM, about 30 mM to about 140 mM, about 30 mM to about 130 mM, about 30 mM to about 120 mM, about 30 mM to about 110 mM, about 30 mM to about 100 mM, about 30 mM to about 90 mM, about 30 mM to about 80 mM, about 30 mM to about 70 mM, about 30 mM to about 60 mM, about 30 mM to about 50 mM, about 30 mM to about 40 mM, about 40 mM to about 150 mM, about 40 mM to about 140 mM, about 40 mM to about 130 mM, about 40 mM to about 120 mM, about 40 mM to about 110 mM, about 40 mM to about 100 mM, about 40 mM to about 90 mM, about 40 mM to about 80 mM, about 40 mM to about 70 mM, about 40 mM to about 60 mM, about 40 mM to about 50 mM, about 50 mM to about 150 mM, about 50 mM to about 140 mM, about 50 mM to about 130 mM, about 50 mM to about 120 mM, about 50 mM to about 110 mM, about 50 mM to about 100 mM, about 50 mM to about 90 mM, about 50 mM to about 80 mM, about 50 mM to about 70 mM, about 50 mM to about 60 mM, about 60 mM to about 150 mM, about 60 mM to about 140 mM, about 60 mM to about 130 mM, about 60 mM to about 120 mM, about 60 mM to about 110 mM, about 60 mM to about 100 mM, about 60 mM to about 90 mM, about 60 mM to about 80 mM, about 60 mM to about 70 mM, about 70 mM to about 150 mM, about 70 mM to about 140 mM, about 70 mM to about 130 mM, about 70 mM to about 120 mM, about 70 mM to about 110 mM, about 70 mM to about 100 mM, about 70 mM to about 90 mM, about 70 mM to about 80 mM, about 80 mM to about 150 mM, about 80 mM to about 140 mM, about 80 mM to about 130 mM, about 80 mM to about 120 mM, about 80 mM to about 110 mM, about 80 mM to about 100 mM, about 80 mM to about 90 mM, about 90 mM to about 150 mM, about 90 mM to about 140 mM, about 90 mM to about 130 mM, about 90 mM to about 120 mM, about 90 mM to about 110 mM, about 90 mM to about 100 mM, about 100 mM to about 150 mM, about 100 mM to about 140 mM, about 100 mM to about 130 mM, about 100 mM to about 120 mM, about 100 mM to about 110 mM, about 110 mM to about 150 mM, about 110 mM to about 140 mM, about 110 mM to about 130 mM, about 110 mM to about 120 mM, about 120 mM to about 150 mM, about 120 mM to about 140 mM, about 120 mM to about 130 mM, about 130 mM to about 150 mM, about 130 mM to about 140 mM, or about 140 mM to about 150 mM.

Non-limiting examples of anionic cell impermeant agents include lactobionate, citrate, and gluconate. Additional examples of anionic cell impermeant agents are known in the art.

### Chloride Ion

The final concentration of chloride ion in any of the pharmaceutically acceptable aqueous buffered solutions described herein can be, e.g., about 0.5 mM to about 60 mM, about 0.5 mM to about 55 mM, about 0.5 mM to about 50 mM, about 0.5 mM to about 45 mM, about 0.5 mM to about 40 mM, about 0.5 mM to about 35 mM, about 0.5 mM to about 30 mM, about 0.5 mM to about 25 mM, about 0.5 mM to about 20 mM, about 0.5 mM to about 15 mM, about 0.5 mM to about 10 mM, about 0.5 mM to about 5 mM, about 0.5 mM to about 1 mM, about 1 mM to about 60 mM, about 1 mM to about 55 mM, about 1 mM to about 50 mM, about 1 mM to about 45 mM, about 1 mM to about 40 mM, about 1 mM to about 35 mM, about 1 mM to about 30 mM, about 1 mM to about 25 mM, about 1 mM to about 20 mM, about 1 mM to about 15 mM, about 1 mM to about 10 mM, about 1 mM to about 5 mM, about 5 mM to about 60 mM, about 5 mM to about 55 mM, about 5 mM to about 50 mM, about 5 mM to about 45 mM, about 5 mM to about 40 mM, about 5 mM to about 35 mM, about 5 mM to about 30 mM, about 5 mM to about 25 mM, about 5 mM to about 20 mM, about 5 mM to about 15 mM, about 5 mM to about 10 mM, about 10 mM to about 60 mM, about 10 mM to about 55 mM, about 10 mM to about 50 mM, about 10 mM to about 45 mM, about 10 mM to about 40 mM, about 10 mM to about 35 mM, about 10 mM to about 30 mM, about 10 mM to about 25 mM, about 10 mM to about 20 mM, about 10 mM to about 15 mM, about 15 mM to about 60 mM, about 15 mM to about 55 mM, about 15 mM to about 50 mM, about 15 mM to about 45 mM, about 15 mM to about 40 mM, about 15 mM to about 35 mM, about 15 mM to about 30 mM, about 15 mM to about 25 mM, about 15 mM to about 20 mM, about 20 mM to about 60 mM, about 20 mM to about 55 mM, about 20 mM to about 50 mM, about 20 mM to about 45 mM, about 20 mM to about 40 mM, about 20 mM to about 35 mM, about 20 mM to about 30 mM, about 20 mM to about 25 mM, about 25 mM to about 60 mM, about 25 mM to about 55 mM, about 25 mM to about 50 mM, about 25 mM to about 45 mM, about 25 mM to about 40 mM, about 25 mM to about 35 mM, about 25 mM to about 30 mM, about 30 mM to about 60 mM, about 30 mM to about 55 mM, about 30 mM to about 50 mM, about 30 mM to about 45 mM, about 30 mM to about 40 mM, about 30 mM to about 35 mM, about 35 mM to about 60 mM, about 35 mM to about 55 mM, about 35 mM to about 50 mM, about 35 mM to about 45 mM, about 35 mM to about 40 mM, about 40 mM to about 60 mM, about 40 mM to about 55 mM, about 40 mM to about 50 mM, about 40 mM to about 45 mM, about 45 mM to about 60 mM, about 45 mM to about 55 mM, about 45 mM to about 50 mM, about 50 mM to about 60 mM, about 50 mM to about 55 mM, or about 55 mM to about 60 mM. Non-limiting sources of sodium ion include different sodium salts, e.g., sodium chloride, potassium chloride, calcium chloride, and magnesium chloride. Additional pharmaceutically acceptable sources of sodium ion are known in the art. In some examples, the sodium ion can be present in the composition as a counterion for one or more of the other anions present in the composition.

### Nucleobase

In some embodiments, the pharmaceutically acceptable aqueous buffered solutions described herein can further include about 0.01 mM to about 10 mM (e.g., about 0.01 mM to about 9 mM, about 0.01 mM to about 8 mM, about 0.01 mM to about 7 mM, about 0.01 mM to about 6 mM, about 0.01 mM to about 5 mM, about 0.01 mM to about 4 mM, about 0.01 mM to about 3 mM, about 0.01 mM to about 2 mM, about 0.01 mM to about 1 mM, about 0.01 mM to about 0.1 mM, about 0.1 mM to about 10 mM, about 0.1 mM to about 9 mM, about 0.1 mM to about 8 mM, about 0.1 mM to about 7 mM, about 0.1 mM to about 6 mM, about 0.1 mM to about 5 mM, about 0.1 mM to about 4 mM, about 0.1 mM to about 3 mM, about 0.1 mM to about 2 mM, about 0.1 mM to about 1 mM, about 1 mM to about 10 mM, about 1 mM to about 9 mM, about 1 mM to about 8 mM, about 1 mM to about 7 mM, about 1 mM to about 6 mM, about 1 mM to about 5 mM, about 1 mM to about 4 mM, about 1 mM to about 3 mM, about 1 mM to about 2 mM, about 2 mM to about 10 mM, about 2 mM to about 9 mM, about 2 mM to about 8 mM, about 2 mM to about 7 mM, about 2 mM to about 6 mM, about 2 mM to about 5 mM, about 2 mM to about 4 mM, about 2 mM to about 3 mM, about 3 mM to about 10 mM, about 3 mM to about 9 mM, about 3 mM to about 8 mM, about 3 mM to about 7 mM, about 3 mM to about 6 mM, about 3 mM to about 5 mM, about 3 mM to about 4 mM, about 4 mM to about 10 mM, about 4 mM to about 9 mM, about 4 mM to about 8 mM, about 4 mM to about 7 mM, about 4 mM to about 6 mM, about 4 mM to about 5 mM, about 5 mM to about 10 mM, about 5 mM to about 9 mM, about 5 mM to about 8 mM, about 5 mM to about 7 mM, about 5 mM to about 6 mM, about 6 mM to about 10 mM, about 6 mM to about 9 mM, about 6 mM to about 8 mM, about 6 mM to about 7 mM, about 7 mM to about 10 mM, about 7 mM to about 9 mM, about 7 mM to about 8 mM, about 8 mM to about 10 mM, about 8 mM to about 9 mM, or about 9 mM to about 10 mM) of a nucleobase. Non-limiting examples of nucleobases include adenine, cytosine, guanine, thymine, hypoxanthine, and uracil.

### Nucleoside

In some embodiments, the pharmaceutically acceptable aqueous buffered solutions described herein can further include about 0.01 mM to about 10 mM (e.g., about 0.01 mM to about 9 mM, about 0.01 mM to about 8 mM, about 0.01 mM to about 7 mM, about 0.01 mM to about 6 mM, about 0.01 mM to about 5 mM, about 0.01 mM to about 4 mM, about 0.01 mM to about 3 mM, about 0.01 mM to about 2 mM, about 0.01 mM to about 1 mM, about 0.01 mM to about 0.1 mM, about 0.1 mM to about 10 mM, about 0.1 mM to about 9 mM, about 0.1 mM to about 8 mM, about 0.1 mM to about 7 mM, about 0.1 mM to about 6 mM, about 0.1 mM to about 5 mM, about 0.1 mM to about 4 mM, about 0.1 mM to about 3 mM, about 0.1 mM to about 2 mM, about 0.1 mM to about 1 mM, about 1 mM to about 10 mM, about 1 mM to about 9 mM, about 1 mM to about 8 mM, about 1 mM to about 7 mM, about 1 mM to about 6 mM, about 1 mM to about 5 mM, about 1 mM to about 4 mM, about 1 mM to about 3 mM, about 1 mM to about 2 mM, about 2 mM to about 10 mM, about 2 mM to about 9 mM, about 2 mM to about 8 mM, about 2 mM to about 7 mM, about 2 mM to about 6 mM, about 2 mM to about 5 mM, about 2 mM to about 4 mM, about 2 mM to about 3 mM, about 3 mM to about 10 mM, about 3 mM to about 9 mM, about 3 mM to about 8 mM, about 3 mM to about 7 mM, about 3 mM to about 6 mM, about 3 mM to about 5 mM, about 3 mM to about 4 mM, about 4 mM to about 10 mM, about 4 mM to about 9 mM, about 4 mM to about 8 mM, about 4 mM to about 7 mM, about 4 mM to about 6 mM, about 4 mM to about 5 mM, about 5 mM to about 10 mM, about 5 mM to about 9 mM, about 5 mM to about 8 mM, about 5 mM to about 7 mM, about 5 mM to about 6 mM, about 6 mM to about 10 mM, about 6 mM to about 9 mM, about 6 mM to about 8 mM, about 6 mM to about 7 mM, about 7 mM to about 10 mM, about 7 mM to about 9 mM, about 7 mM to about 8 mM, about 8 mM to about 10 mM, about 8 mM to about 9 mM, or about 9 mM to about 10 mM) of a nucleoside. Non-limiting examples of nucleosides include adenosine, inosine, cytidine, uridine, guanosine, and thymidine.

### Nucleotide

In some embodiments, the pharmaceutically acceptable aqueous buffered solutions described herein can further include about 0.01 mM to about 10 mM (e.g., about 0.01 mM to about 9 mM, about 0.01 mM to about 8 mM, about 0.01 mM to about 7 mM, about 0.01 mM to about 6 mM, about 0.01 mM to about 5 mM, about 0.01 mM to about 4 mM, about 0.01 mM to about 3 mM, about 0.01 mM to about 2 mM, about 0.01 mM to about 1 mM, about 0.01 mM to about 0.1 mM, about 0.1 mM to about 10 mM, about 0.1 mM to about 9 mM, about 0.1 mM to about 8 mM, about 0.1 mM to about 7 mM, about 0.1 mM to about 6 mM, about 0.1 mM to about 5 mM, about 0.1 mM to about 4 mM, about 0.1 mM to about 3 mM, about 0.1 mM to about 2 mM, about 0.1 mM to about 1 mM, about 1 mM to about 10 mM, about 1 mM to about 9 mM, about 1 mM to about 8 mM, about 1 mM to about 7 mM, about 1 mM to about 6 mM, about 1 mM to about 5 mM, about 1 mM to about 4 mM, about 1 mM to about 3 mM, about 1 mM to about 2 mM, about 2 mM to about 10 mM, about 2 mM to about 9 mM, about 2 mM to about 8 mM, about 2 mM to about 7 mM, about 2 mM to about 6 mM, about 2 mM to about 5 mM, about 2 mM to about 4 mM, about 2 mM to about 3 mM, about 3 mM to about 10 mM, about 3 mM to about 9 mM, about 3 mM to about 8 mM, about 3 mM to about 7 mM, about 3 mM to about 6 mM, about 3 mM to about 5 mM, about 3 mM to about 4 mM, about 4 mM to about 10 mM, about 4 mM to about 9 mM, about 4 mM to about 8 mM, about 4 mM to about 7 mM, about 4 mM to about 6 mM, about 4 mM to about 5 mM, about 5 mM to about 10 mM, about 5 mM to about 9 mM, about 5 mM to about 8 mM, about 5 mM to about 7 mM, about 5 mM to about 6 mM, about 6 mM to about 10 mM, about 6 mM to about 9 mM, about 6 mM to about 8 mM, about 6 mM to about 7 mM, about 7 mM to about 10 mM, about 7 mM to about 9 mM, about 7 mM to about 8 mM, about 8 mM to about 10 mM, about 8 mM to about 9 mM, or about 9 mM to about 10 mM) of a nucleotide. Non-limiting examples of nucleotides include adenosine monophosphate, adenosine diphosphate, adenosine triphosphate, guanosine monophosphate, guanosine diphosphate, guanosine triphosphate, cytidine monophosphate, cytidine diphosphate, cytidine triphosphate, thymidine monophosphate, thymidine diphosphate, thymidine triphosphate, uridine monophosphate, uridine diphosphate, and uridine triphosphate.

### Bicarbonate Ion

In some embodiments, the pharmaceutically acceptable aqueous buffered solutions described herein can further include about 0.01 mM to about 20 mM (e.g., about 0.01 mM to about 18 mM, about 0.01 mM to about 16 mM, about 0.01 mM to about 14 mM, about 0.01 mM to about 12 mM, about 0.01 mM to about 10 mM, about 0.01 mM to about 9 mM, about 0.01 mM to about 8 mM, about 0.01 mM to about 7 mM, about 0.01 mM to about 6 mM, about 0.01 mM to about 5 mM, about 0.01 mM to about 4 mM, about 0.01 mM to about 3 mM, about 0.01 mM to about 2 mM, about 0.01 mM to about 1 mM, about 1 mM to about 20 mM, about 1 mM to about 18 mM, about 1 mM to about 16 mM, about 1 mM to about 14 mM, about 1 mM to about 12 mM, about 1 mM to about 10 mM, about 1 mM to about 9 mM, about 1 mM to about 8 mM, about 1 mM to about 7 mM, about 1 mM to about 6 mM, about 1 mM to about 5 mM, about 1 mM to about 4 mM, about 1 mM to about 3 mM, about 1 mM to about 2 mM, about 2 mM to about 20 mM, about 2 mM to about 18 mM, about 2 mM to about 16 mM, about 2 mM to about 14 mM, about 2 mM to about 12 mM, about 2 mM to about 10 mM, about 2 mM to about 9 mM, about 2 mM to about 8 mM, about 2 mM to about 7 mM, about 2 mM to about 6 mM, about 2 mM to about 5 mM, about 2 mM to about 4 mM, about 2 mM to about 3 mM, about 3 mM to about 20 mM, about 3 mM to about 18 mM, about 3 mM to about 16 mM, about 3 mM to about 14 mM, about 3 mM to about 12 mM, about 3 mM to about 10 mM, about 3 mM to about 9 mM, about 3 mM to about 8 mM, about 3 mM to about 7 mM, about 3 mM to about 6 mM, about 3 mM to about 5 mM, about 3 mM to about 4 mM, about 4 mM to about 20 mM, about 4 mM to about 18 mM, about 4 mM to about 16 mM, about 4 mM to about 14 mM, about 4 mM to about 12 mM, about 4 mM to about 10 mM, about 4 mM to about 9 mM, about 4 mM to about 8 mM, about 4 mM to about 7 mM, about 4 mM to about 6 mM, about 4 mM to about 5 mM, about 5 mM to about 20 mM, about 5 mM to about 18 mM, about 5 mM to about 16 mM, about 5 mM to about 14 mM, about 5 mM to about 12 mM, about 5 mM to about 10 mM, about 5 mM to about 9 mM, about 5 mM to about 8 mM, about 5 mM to about 7 mM, about 5 mM to about 6 mM, about 6 mM to about 20 mM, about 6 mM to about 18 mM, about 6 mM to about 16 mM, about 6 mM to about 14 mM, about 6 mM to about 12 mM, about 6 mM to about 10 mM, about 6 mM to about 9 mM, about 6 mM to about 8 mM, about 6 mM to about 7 mM, about 7 mM to about 20 mM, about 7 mM to about 18 mM, about 7 mM to about 16 mM, about 7 mM to about 14 mM, about 7 mM to about 12 mM, about 7 mM to about 10 mM, about 7 mM to about 9 mM, about 7 mM to about 8 mM, about 8 mM to about 20 mM, about 8 mM to about 18 mM, about 8 mM to about 16 mM, about 8 mM to about 14 mM, about 8 mM to about 12 mM, about 8 mM to about 10 mM, about 8 mM to about 9 mM, about 9 mM to about 20 mM, about 9 mM to about 18 mM, about 9 mM to about 16 mM, about 9 mM to about 14 mM, about 9 mM to about 12 mM, about 9 mM to about 10 mM, about 10 mM to about 20 mM, about 10 mM to about 18 mM, about 10 mM to about 16 mM, about 10 mM to about 14 mM, about 10 mM to about 12 mM, about 12 mM to about 20 mM, about 12 mM to about 18 mM, about 12 mM to about 16 mM, about 12 mM to about 14 mM, about 14 mM to about 20 mM, about 14 mM to about 18 mM, about 14 mM to about 16 mM, about 16 mM to about 20 mM, about 16 mM to about 18 mM, or about 18 mM to about 20 mM) bicarbonate ion.

In some embodiments, the biocarbonate ion can be present in the pharmaceutically acceptable aqueous buffered solution as sodium bicarbonate. Additional pharmaceutically acceptable sources of bicarbonate ion are known in the art.

### Pyruvate

In some embodiments, the pharmaceutically acceptable aqueous buffered solutions described herein can further include about 0.01 mM to about 10 mM (e.g., about 0.01 mM to about 9 mM, about 0.01 mM to about 8 mM, about 0.01 mM to about 7 mM, about 0.01 mM to about 6 mM, about 0.01 mM to about 5 mM, about 0.01 mM to about 4 mM, about 0.01 mM to about 3 mM, about 0.01 mM to about 2 mM, about 0.01 mM to about 1 mM, about 0.01 mM to about 0.1 mM, about 0.1 mM to about 10 mM, about 0.1 mM to about 9 mM, about 0.1 mM to about 8 mM, about 0.1 mM to about 7 mM, about 0.1 mM to about 6 mM, about 0.1 mM to about 5 mM, about 0.1 mM to about 4 mM, about 0.1 mM to about 3 mM, about 0.1 mM to about 2 mM, about 0.1 mM to about 1 mM, about 1 mM to about 10 mM, about 1 mM to about 9 mM, about 1 mM to about 8 mM, about 1 mM to about 7 mM, about 1 mM to about 6 mM, about 1 mM to about 5 mM, about 1 mM to about 4 mM, about 1 mM to about 3 mM, about 1 mM to about 2 mM, about 2 mM to about 10 mM, about 2 mM to about 9 mM, about 2 mM to about 8 mM, about 2 mM to about 7 mM, about 2 mM to about 6 mM, about 2 mM to about 5 mM, about 2 mM to about 4 mM, about 2 mM to about 3 mM, about 3 mM to about 10 mM, about 3 mM to about 9 mM, about 3 mM to about 8 mM, about 3 mM to about 7 mM, about 3 mM to about 6 mM, about 3 mM to about 5 mM, about 3 mM to about 4 mM, about 4 mM to about 10 mM, about 4 mM to about 9 mM, about 4 mM to about 8 mM, about 4 mM to about 7 mM, about 4 mM to about 6 mM, about 4 mM to about 5 mM, about 5 mM to about 10 mM, about 5 mM to about 9 mM, about 5 mM to about 8 mM, about 5 mM to about 7 mM, about 5 mM to about 6 mM, about 6 mM to about 10 mM, about 6 mM to about 9 mM, about 6 mM to about 8 mM, about 6 mM to about 7 mM, about 7 mM to about 10 mM, about 7 mM to about 9 mM, about 7 mM to about 8 mM, about 8 mM to about 10 mM, about 8 mM to about 9 mM, or about 9 mM to about 10 mM) pyruvate.

### Poloxamer

In some embodiments, the pharmaceutical acceptable aqueous buffered solutions described herein can further include a poloxamer (e.g., poloxamer-188). The final concentration of a poloxamer (e.g., poloxamer-124, poloxamer-182, poloxamer-188, poloxamer-331, and/or poloxamer-407) in any of the pharmaceutically acceptable aqueous buffered solutions described herein can be about 0.01% w/v to about 2.0% w/v (e.g., about 0.01% w/v to about 1.9% w/v, about 0.01% w/v to about 1.8% w/v, about 0.01% w/v to about 1.7% w/v, about 0.01% w/v to about 1.6% w/v, about 0.01% w/v to about 1.5% w/v, about 0.01% w/v to about 1.4% w/v, about 0.01% w/v to about 1.3% w/v, about 0.01% w/v to about 1.2% w/v, about 0.01% w/v to about 1.1% w/v, about 0.01% w/v to about 1.0% w/v, about 0.01% w/v to about 0.90% w/v, about 0.01% w/v to about 0.80% w/v, about 0.01% w/v to about 0.75% w/v, about 0.01% w/v to about 0.70% w/v, about 0.01% w/v to about 0.65% w/v, about 0.01% w/v to about 0.60% w/v, about 0.01% w/v to about 0.55% w/v, about 0.01% w/v to about 0.50% w/v, about 0.01% w/v to about 0.45% w/v, about 0.01% w/v to about 0.40% w/v, about 0.01% w/v to about 0.35% w/v, about 0.01% w/v to about 0.30% w/v, about 0.01% w/v to about 0.25% w/v, about 0.01% w/v to about 0.20% w/v, about 0.01% w/v to about 0.15% w/v, about 0.01% w/v to about 0.10% w/v, about 0.01% to about 0.05% w/v, about 0.05% w/v to about 2.0% w/v, about 0.05% w/v to about 1.9% w/v, about 0.05% w/v to about 1.8% w/v, about 0.05% w/v to about 1.7% w/v, about 0.05% w/v to about 1.6% w/v, about 0.05% w/v to about 1.5% w/v, about 0.05% w/v to about 1.4% w/v, about 0.05% w/v to about 1.3% w/v, about 0.05% w/v to about 1.2% w/v, about 0.05% w/v to about 1.1% w/v, about 0.05% w/v to about 1.0% w/v, about 0.05% w/v to about 0.90% w/v, about 0.05% w/v to about 0.80% w/v, about 0.05% w/v to about 0.75% w/v, about 0.05% w/v to about 0.70% w/v, about 0.05% w/v to about 0.65% w/v, about 0.05% w/v to about 0.60% w/v, about 0.05% w/v to about 0.55% w/v, about 0.05% w/v to about 0.50% w/v, about 0.05% w/v to about 0.45% w/v, about 0.05% w/v to about 0.40% w/v, about 0.05% w/v to about 0.35% w/v, about 0.05% w/v to about 0.30% w/v, about 0.05% w/v to about 0.25% w/v, about 0.05% w/v to about 0.20% w/v, about 0.05% w/v to about 0.15% w/v, about 0.05% w/v to about 0.10% w/v, about 0.10% w/v to about 2.0% w/v, about 0.10% w/v to about 1.9% w/v, about 0.10% w/v to about 1.8% w/v, about 0.10% w/v to about 1.7% w/v, about 0.10% w/v to about 1.6% w/v, about 0.10% w/v to about 1.5% w/v, about 0.10% w/v to about 1.4% w/v, about 0.10% w/v to about 1.3% w/v, about 0.10% w/v to about 1.2% w/v, about 0.10% w/v to about 1.1% w/v, about 0.10% w/v to about 1.0% w/v, about 0.10% w/v to about 0.90% w/v, about 0.10% w/v to about 0.80% w/v, about 0.10% w/v to about 0.75% w/v, about 0.10% w/v to about 0.70% w/v, about 0.10% w/v to about 0.65% w/v, about 0.10% w/v to about 0.60% w/v, about 0.10% w/v to about 0.55% w/v, about 0.10% w/v to about 0.50% w/v, about 0.10% w/v to about 0.45% w/v, about 0.10% w/v to about 0.40% w/v, about 0.10% w/v to about 0.35% w/v, about 0.10% w/v to about 0.30% w/v, about 0.10% w/v to about 0.25% w/v, about 0.10% w/v to about 0.20% w/v, about 0.10% w/v to about 0.15% w/v, about 0.15% w/v to about 2.0% w/v, about 0.15% w/v to about 1.9% w/v, about 0.15% w/v to about 1.8% w/v, about 0.15% w/v to about 1.7% w/v, about 0.15% w/v to about 1.6% w/v, about 0.15% w/v to about 1.5% w/v, about 0.15% w/v to about 1.4% w/v, about 0.15% w/v to about 1.3% w/v, about 0.15% w/v to about 1.2% w/v, about 0.15% w/v to about 1.1% w/v, about 0.15% w/v to about 1.0% w/v, about 0.15% w/v to about 0.90% w/v, about 0.15% w/v to about 0.80% w/v, about 0.15% w/v to about 0.75% w/v, about 0.15% w/v to about 0.70% w/v, about 0.15% w/v to about 0.65% w/v, about 0.15% w/v to about 0.60% w/v, about 0.15% w/v to about 0.55% w/v, about 0.15% w/v to about 0.50% w/v, about 0.15% w/v to about 0.45% w/v, about 0.15% w/v to about 0.40% w/v, about 0.15% w/v to about 0.35% w/v, about 0.15% w/v to about 0.30% w/v, about 0.15% w/v to about 0.25% w/v, about 0.15% w/v to about 0.20% w/v, about 0.20% w/v to about 2.0% w/v, about 0.20% w/v to about 1.9% w/v, about 0.20% w/v to about 1.8% w/v, about 0.20% w/v to about 1.7% w/v, about 0.20% w/v to about 1.6% w/v, about 0.20% w/v to about 1.5% w/v, about 0.20% w/v to about 1.4% w/v, about 0.20% w/v to about 1.3% w/v, about 0.20% w/v to about 1.2% w/v, about 0.20% w/v to about 1.1% w/v, about 0.20% w/v to about 1.0% w/v, about 0.20% w/v to about 0.90% w/v, about 0.20% w/v to about 0.80% w/v, about 0.20% w/v to about 0.75% w/v, about 0.20% w/v to about 0.70% w/v, about 0.20% w/v to about 0.65% w/v, about 0.20% w/v to about 0.60% w/v, about 0.20% w/v to about 0.55% w/v, about 0.20% w/v to about 0.50% w/v, about 0.20% w/v to about 0.45% w/v, about 0.20% w/v to about 0.40% w/v, about 0.20% w/v to about 0.35% w/v, about 0.20% w/v to about 0.30% w/v, about 0.20% w/v to about 0.25% w/v, about 0.25% w/v to about 2.0% w/v, about 0.25% w/v to about 1.9% w/v, about 0.25% w/v to about 1.8% w/v, about 0.25% w/v to about 1.7% w/v, about 0.25% w/v to about 1.6% w/v, about 0.25% w/v to about 1.5% w/v, about 0.25% w/v to about 1.4% w/v, about 0.25% w/v to about 1.3% w/v, about 0.25% w/v to about 1.2% w/v, about 0.25% w/v to about 1.1% w/v, about 0.25% w/v to about 1.0% w/v, about 0.25% w/v to about 0.90% w/v, about 0.25% w/v to about 0.80% w/v, about 0.25% w/v to about 0.75% w/v, about 0.25% w/v to about 0.70% w/v, about 0.25% w/v to about 0.65% w/v, about 0.25% w/v to about 0.60% w/v, about 0.25% w/v to about 0.55% w/v, about 0.25% w/v to about 0.50% w/v, about 0.25% w/v to about 0.45% w/v, about 0.25% w/v to about 0.40% w/v, about 0.25% w/v to about 0.35% w/v, about 0.25% w/v to about 0.30% w/v, about 0.30% w/v to about 2.0% w/v, about 0.30% w/v to about 1.9% w/v, about 0.30% w/v to about 1.8% w/v, about 0.30% w/v to about 1.7% w/v, about 0.30% w/v to about 1.6% w/v, about 0.30% w/v to about 1.5% w/v, about 0.30% w/v to about 1.4% w/v, about 0.30% w/v to about 1.3% w/v, about 0.30% w/v to about 1.2% w/v, about 0.30% w/v to about 1.1% w/v, about 0.30% w/v to about 1.0% w/v, about 0.30% w/v to about 0.90% w/v, about 0.30% w/v to about 0.80% w/v, about 0.30% w/v to about 0.75% w/v, about 0.30% w/v to about 0.70% w/v, about 0.30% w/v to about 0.65% w/v, about 0.30% w/v to about 0.60% w/v, about 0.30% w/v to about 0.55% w/v, about 0.30% w/v to about 0.50% w/v, about 0.30% w/v to about 0.45% w/v, about 0.30% w/v to about 0.40% w/v, about 0.30% w/v to about 0.35% w/v, about 0.35% w/v to about 2.0% w/v, about 0.35% w/v to about 1.9% w/v, about 0.35% w/v to about 1.8% w/v, about 0.35% w/v to about 1.7% w/v, about 0.35% w/v to about 1.6% w/v, about 0.35% w/v to about 1.5% w/v, about 0.35% w/v to about 1.4% w/v, about 0.35% w/v to about 1.3% w/v, about 0.35% w/v to about 1.2% w/v, about 0.35% w/v to about 1.1% w/v, about 0.35% w/v to about 1.0% w/v, about 0.35% w/v to about 0.90% w/v, about 0.35% w/v to about 0.80% w/v, about 0.35% w/v to about 0.75% w/v, about 0.35% w/v to about 0.70% w/v, about 0.35% w/v to about 0.65% w/v, about 0.35% w/v to about 0.60% w/v, about 0.35% w/v to about 0.55% w/v, about 0.35% w/v to about 0.50% w/v, about 0.35% w/v to about 0.45% w/v, about 0.35% w/v to about 0.40% w/v, about 0.40% w/v to about 2.0% w/v, about 0.40% w/v to about 1.9% w/v, about 0.40% w/v to about 1.8% w/v, about 0.40% w/v to about 1.7% w/v, about 0.40% w/v to about 1.6% w/v, about 0.40% w/v to about 1.5% w/v, about 0.40% w/v to about 1.4% w/v, about 0.40% w/v to about 1.3% w/v, about 0.40% w/v to about 1.2% w/v, about 0.40% w/v to about 1.1% w/v, about 0.40% w/v to about 1.0% w/v, about 0.40% w/v to about 0.90% w/v, about 0.40% w/v to about 0.80% w/v, about 0.40% w/v to about 0.75% w/v, about 0.40% w/v to about 0.70% w/v, about 0.40% w/v to about 0.65% w/v, about 0.40% w/v to about 0.60% w/v, about 0.40% w/v to about 0.55% w/v, about 0.40% w/v to about 0.50% w/v, about 0.40% w/v to about 0.45% w/v, about 0.45% w/v to about 2.0% w/v, about 0.45% w/v to about 1.9% w/v, about 0.45% w/v to about 1.8% w/v, about 0.45% w/v to about 1.7% w/v, about 0.45% w/v to about 1.6% w/v, about 0.45% w/v to about 1.5% w/v, about 0.45% w/v to about 1.4% w/v, about 0.45% w/v to about 1.3% w/v, about 0.45% w/v to about 1.2% w/v, about 0.45% w/v to about 1.1% w/v, about 0.45% w/v to about 1.0% w/v, about 0.45% w/v to about 0.90% w/v, about 0.45% w/v to about 0.80% w/v, about 0.45% w/v to about 0.75% w/v, about 0.45% w/v to about 0.70% w/v, about 0.45% w/v to about 0.65% w/v, about 0.45% w/v to about 0.60% w/v, about 0.45% w/v to about 0.55% w/v, about 0.45% w/v to about 0.50% w/v, about 0.50% w/v to about 2.0% w/v, about 0.50% w/v to about 1.9% w/v, about 0.50% w/v to about 1.8% w/v, about 0.50% w/v to about 1.7% w/v, about 0.50% w/v to about 1.6% w/v, about 0.50% w/v to about 1.5% w/v, about 0.50% w/v to about 1.4% w/v, about 0.50% w/v to about 1.3% w/v, about 0.50% w/v to about 1.2% w/v, about 0.50% w/v to about 1.1% w/v, about 0.50% w/v to about 1.0% w/v, about 0.50% w/v to about 0.90% w/v, about 0.50% w/v to about 0.80% w/v, about 0.50% w/v to about 0.75% w/v, about 0.50% w/v to about 0.70% w/v, about 0.50% w/v to about 0.65% w/v, about 0.50% w/v to about 0.60% w/v, about 0.50% w/v to about 0.55% w/v, about 0.55% w/v to about 2.0% w/v, about 0.55% w/v to about 1.9% w/v, about 0.55% w/v to about 1.8% w/v, about 0.55% w/v to about 1.7% w/v, about 0.55% w/v to about 1.6% w/v, about 0.55% w/v to about 1.5% w/v, about 0.55% w/v to about 1.4% w/v, about 0.55% w/v to about 1.3% w/v, about 0.55% w/v to about 1.2% w/v, about 0.55% w/v to about 1.1% w/v, about 0.55% w/v to about 1.0% w/v, about 0.55% w/v to about 0.90% w/v, about 0.55% w/v to about 0.80% w/v, about 0.55% w/v to about 0.75% w/v, about 0.55% w/v to about 0.70% w/v, about 0.55% w/v to about 0.65% w/v, about 0.55% w/v to about 0.60% w/v, about 0.60% w/v to about 2.0% w/v, about 0.60% w/v to about 1.9% w/v, about 0.60% w/v to about 1.8% w/v, about 0.60% w/v to about 1.7% w/v, about 0.60% w/v to about 1.6% w/v, about 0.60% w/v to about 1.5% w/v, about 0.60% w/v to about 1.4% w/v, about 0.60% w/v to about 1.3% w/v, about 0.60% w/v to about 1.2% w/v, about 0.60% w/v to about 1.1% w/v, about 0.60% w/v to about 1.0% w/v, about 0.60% w/v to about 0.90% w/v, about 0.60% w/v to about 0.80% w/v, about 0.60% w/v to about 0.75% w/v, about 0.60% w/v to about 0.70% w/v, about 0.60% w/v to about 0.65% w/v, about 0.65% w/v to about 2.0% w/v, about 0.65% w/v to about 1.9% w/v, about 0.65% w/v to about 1.8% w/v, about 0.65% w/v to about 1.7% w/v, about 0.65% w/v to about 1.6% w/v, about 0.65% w/v to about 1.5% w/v, about 0.65% w/v to about 1.4% w/v, about 0.65% w/v to about 1.3% w/v, about 0.65% w/v to about 1.2% w/v, about 0.65% w/v to about 1.1% w/v, about 0.65% w/v to about 1.0% w/v, about 0.65% w/v to about 0.90% w/v, about 0.65% w/v to about 0.80% w/v, about 0.65% w/v to about 0.75% w/v, about 0.65% w/v to about 0.70% w/v, about 0.70% w/v to about 2.0% w/v, about 0.70% w/v to about 1.9% w/v, about 0.70% w/v to about 1.8% w/v, about 0.70% w/v to about 1.7% w/v, about 0.70% w/v to about 1.6% w/v, about 0.70% w/v to about 1.5% w/v, about 0.70% w/v to about 1.4% w/v, about 0.70% w/v to about 1.3% w/v, about 0.70% w/v to about 1.2% w/v, about 0.70% w/v to about 1.1% w/v, about 0.70% w/v to about 1.0% w/v, about 0.70% w/v to about 0.90% w/v, about 0.70% w/v to about 0.80% w/v, about 0.70% w/v to about 0.75% w/v, about 0.75% w/v to about 2.0% w/v, about 0.75% w/v to about 1.9% w/v, about 0.75% w/v to about 1.8% w/v, about 0.75% w/v to about 1.7% w/v, about 0.75% w/v to about 1.6% w/v, about 0.75% w/v to about 1.5% w/v, about 0.75% w/v to about 1.4% w/v, about 0.75% w/v to about 1.3% w/v, about 0.75% w/v to about 1.2% w/v, about 0.75% w/v to about 1.1% w/v, about 0.75% w/v to about 1.0% w/v, about 0.75% w/v to about 0.90% w/v, about 0.75% w/v to about 0.80% w/v, about 0.80% w/v to about 2.0% w/v, about 0.80% w/v to about 1.9% w/v, about 0.80% w/v to about 1.8% w/v, about 0.80% w/v to about 1.7% w/v, about 0.80% w/v to about 1.6% w/v, about 0.80% w/v to about 1.5% w/v, about 0.80% w/v to about 1.4% w/v, about 0.80% w/v to about 1.3% w/v, about 0.80% w/v to about 1.2% w/v, about 0.80% w/v to about 1.1% w/v, about 0.80% w/v to about 1.0% w/v, about 0.80% w/v to about 0.90% w/v, about 0.90% w/v to about 2.0% w/v, about 0.90% w/v to about 1.9% w/v, about 0.90% w/v to about 1.8% w/v, about 0.90% w/v to about 1.7% w/v, about 0.90% w/v to about 1.6% w/v, about 0.90% w/v to about 1.5% w/v, about 0.90% w/v to about 1.4% w/v, about 0.90% w/v to about 1.3% w/v, about 0.90% w/v to about 1.2% w/v, about 0.90% w/v to about 1.1% w/v, about 0.90% w/v to about 1.0% w/v, about 1.0% w/v to about 2.0% w/v, about 1.0% w/v to about 1.9% w/v, about 1.0% w/v to about 1.8% w/v, about 1.0% w/v to about 1.7% w/v, about 1.0% w/v to about 1.6% w/v, about 1.0% w/v to about 1.5% w/v, about 1.0% w/v to about 1.4% w/v, about 1.0% w/v to about 1.3% w/v, about 1.0% w/v to about 1.2% w/v, about 1.0% w/v to about 1.1% w/v, about 1.1% w/v to about 2.0% w/v, about 1.1% w/v to about 1.9% w/v, about 1.1% w/v to about 1.8% w/v, about 1.1% w/v to about 1.7% w/v, about 1.1% w/v to about 1.6% w/v, about 1.1% w/v to about 1.5% w/v, about 1.1% w/v to about 1.4% w/v, about 1.1% w/v to about 1.3% w/v, about 1.1% w/v to about 1.2% w/v, about 1.2% w/v to about 2.0% w/v, about 1.2% w/v to about 1.9% w/v, about 1.2% w/v to about 1.8% w/v, about 1.2% w/v to about 1.7% w/v, about 1.2% w/v to about 1.6% w/v, about 1.2% w/v to about 1.5% w/v, about 1.2% w/v to about 1.4% w/v, about 1.2% w/v to about 1.3% w/v, about 1.3% w/v to about 2.0% w/v, about 1.3% w/v to about 1.9% w/v, about 1.3% w/v to about 1.8% w/v, about 1.3% w/v to about 1.7% w/v, about 1.3% w/v to about 1.6% w/v, about 1.3% w/v to about 1.5% w/v, about 1.3% w/v to about 1.4% w/v, about 1.4% w/v to about 2.0% w/v, about 1.4% w/v to about 1.9% w/v, about 1.4% w/v to about 1.8% w/v, about 1.4% w/v to about 1.7% w/v, about 1.4% w/v to about 1.6% w/v, about 1.4% w/v to about 1.5% w/v, about 1.5% w/v to about 2.0% w/v, about 1.5% w/v to about 1.9% w/v, about 1.5% w/v to about 1.8% w/v, about 1.5% w/v to about 1.7% w/v, about 1.5% w/v to about 1.6% w/v, about 1.6% w/v to about 2.0% w/v, about 1.6% w/v to about 1.9% w/v, about 1.6% w/v to about 1.8% w/v, about 1.6% w/v to about 1.7% w/v, about 1.7% w/v to about 2.0% w/v, about 1.7% w/v to about 1.9% w/v, about 1.7% w/v to about 1.8% w/v, about 1.8% w/v to about 2.0% w/v, about 1.8% w/v to about 1.9% w/v, or about 1.9% w/v to about 2.0% w/v).

### Serum Albumin

In some embodiments, the pharmaceutical acceptable aqueous buffered solutions described herein can further include a serum albumin (e.g., human serum albumin or HSA). The final concentration of a serum albumin (e.g., human serum albumin or HSA) in any of the pharmaceutically acceptable aqueous buffered solutions described herein can be about 0.01% w/v to about 2.0% w/v (e.g., about 0.01% w/v to about 1.9% w/v, about 0.01% w/v to about 1.8% w/v, about 0.01% w/v to about 1.7% w/v, about 0.01% w/v to about 1.6% w/v, about 0.01% w/v to about 1.5% w/v, about 0.01% w/v to about 1.4% w/v, about 0.01% w/v to about 1.3% w/v, about 0.01% w/v to about 1.2% w/v, about 0.01% w/v to about 1.1% w/v, about 0.01% w/v to about 1.0% w/v, about 0.01% w/v to about 0.90% w/v, about 0.01% w/v to about 0.80% w/v, about 0.01% w/v to about 0.75% w/v, about 0.01% w/v to about 0.70% w/v, about 0.01% w/v to about 0.65% w/v, about 0.01% w/v to about 0.60% w/v, about 0.01% w/v to about 0.55% w/v, about 0.01% w/v to about 0.50% w/v, about 0.01% w/v to about 0.45% w/v, about 0.01% w/v to about 0.40% w/v, about 0.01% w/v to about 0.35% w/v, about 0.01% w/v to about 0.30% w/v, about 0.01% w/v to about 0.25% w/v, about 0.01% w/v to about 0.20% w/v, about 0.01% w/v to about 0.15% w/v, about 0.01% w/v to about 0.10% w/v, about 0.01% to about 0.05% w/v, about 0.05% w/v to about 2.0% w/v, about 0.05% w/v to about 1.9% w/v, about 0.05% w/v to about 1.8% w/v, about 0.05% w/v to about 1.7% w/v, about 0.05% w/v to about 1.6% w/v, about 0.05% w/v to about 1.5% w/v, about 0.05% w/v to about 1.4% w/v, about 0.05% w/v to about 1.3% w/v, about 0.05% w/v to about 1.2% w/v, about 0.05% w/v to about 1.1% w/v, about 0.05% w/v to about 1.0% w/v, about 0.05% w/v to about 0.90% w/v, about 0.05% w/v to about 0.80% w/v, about 0.05% w/v to about 0.75% w/v, about 0.05% w/v to about 0.70% w/v, about 0.05% w/v to about 0.65% w/v, about 0.05% w/v to about 0.60% w/v, about 0.05% w/v to about 0.55% w/v, about 0.05% w/v to about 0.50% w/v, about 0.05% w/v to about 0.45% w/v, about 0.05% w/v to about 0.40% w/v, about 0.05% w/v to about 0.35% w/v, about 0.05% w/v to about 0.30% w/v, about 0.05% w/v to about 0.25% w/v, about 0.05% w/v to about 0.20% w/v, about 0.05% w/v to about 0.15% w/v, about 0.05% w/v to about 0.10% w/v, about 0.10% w/v to about 2.0% w/v, about 0.10% w/v to about 1.9% w/v, about 0.10% w/v to about 1.8% w/v, about 0.10% w/v to about 1.7% w/v, about 0.10% w/v to about 1.6% w/v, about 0.10% w/v to about 1.5% w/v, about 0.10% w/v to about 1.4% w/v, about 0.10% w/v to about 1.3% w/v, about 0.10% w/v to about 1.2% w/v, about 0.10% w/v to about 1.1% w/v, about 0.10% w/v to about 1.0% w/v, about 0.10% w/v to about 0.90% w/v, about 0.10% w/v to about 0.80% w/v, about 0.10% w/v to about 0.75% w/v, about 0.10% w/v to about 0.70% w/v, about 0.10% w/v to about 0.65% w/v, about 0.10% w/v to about 0.60% w/v, about 0.10% w/v to about 0.55% w/v, about 0.10% w/v to about 0.50% w/v, about 0.10% w/v to about 0.45% w/v, about 0.10% w/v to about 0.40% w/v, about 0.10% w/v to about 0.35% w/v, about 0.10% w/v to about 0.30% w/v, about 0.10% w/v to about 0.25% w/v, about 0.10% w/v to about 0.20% w/v, about 0.10% w/v to about 0.15% w/v, about 0.15% w/v to about 2.0% w/v, about 0.15% w/v to about 1.9% w/v, about 0.15% w/v to about 1.8% w/v, about 0.15% w/v to about 1.7% w/v, about 0.15% w/v to about 1.6% w/v, about 0.15% w/v to about 1.5% w/v, about 0.15% w/v to about 1.4% w/v, about 0.15% w/v to about 1.3% w/v, about 0.15% w/v to about 1.2% w/v, about 0.15% w/v to about 1.1% w/v, about 0.15% w/v to about 1.0% w/v, about 0.15% w/v to about 0.90% w/v, about 0.15% w/v to about 0.80% w/v, about 0.15% w/v to about 0.75% w/v, about 0.15% w/v to about 0.70% w/v, about 0.15% w/v to about 0.65% w/v, about 0.15% w/v to about 0.60% w/v, about 0.15% w/v to about 0.55% w/v, about 0.15% w/v to about 0.50% w/v, about 0.15% w/v to about 0.45% w/v, about 0.15% w/v to about 0.40% w/v, about 0.15% w/v to about 0.35% w/v, about 0.15% w/v to about 0.30% w/v, about 0.15% w/v to about 0.25% w/v, about 0.15% w/v to about 0.20% w/v, about 0.20% w/v to about 2.0% w/v, about 0.20% w/v to about 1.9% w/v, about 0.20% w/v to about 1.8% w/v, about 0.20% w/v to about 1.7% w/v, about 0.20% w/v to about 1.6% w/v, about 0.20% w/v to about 1.5% w/v, about 0.20% w/v to about 1.4% w/v, about 0.20% w/v to about 1.3% w/v, about 0.20% w/v to about 1.2% w/v, about 0.20% w/v to about 1.1% w/v, about 0.20% w/v to about 1.0% w/v, about 0.20% w/v to about 0.90% w/v, about 0.20% w/v to about 0.80% w/v, about 0.20% w/v to about 0.75% w/v, about 0.20% w/v to about 0.70% w/v, about 0.20% w/v to about 0.65% w/v, about 0.20% w/v to about 0.60% w/v, about 0.20% w/v to about 0.55% w/v, about 0.20% w/v to about 0.50% w/v, about 0.20% w/v to about 0.45% w/v, about 0.20% w/v to about 0.40% w/v, about 0.20% w/v to about 0.35% w/v, about 0.20% w/v to about 0.30% w/v, about 0.20% w/v to about 0.25% w/v, about 0.25% w/v to about 2.0% w/v, about 0.25% w/v to about 1.9% w/v, about 0.25% w/v to about 1.8% w/v, about 0.25% w/v to about 1.7% w/v, about 0.25% w/v to about 1.6% w/v, about 0.25% w/v to about 1.5% w/v, about 0.25% w/v to about 1.4% w/v, about 0.25% w/v to about 1.3% w/v, about 0.25% w/v to about 1.2% w/v, about 0.25% w/v to about 1.1% w/v, about 0.25% w/v to about 1.0% w/v, about 0.25% w/v to about 0.90% w/v, about 0.25% w/v to about 0.80% w/v, about 0.25% w/v to about 0.75% w/v, about 0.25% w/v to about 0.70% w/v, about 0.25% w/v to about 0.65% w/v, about 0.25% w/v to about 0.60% w/v, about 0.25% w/v to about 0.55% w/v, about 0.25% w/v to about 0.50% w/v, about 0.25% w/v to about 0.45% w/v, about 0.25% w/v to about 0.40% w/v, about 0.25% w/v to about 0.35% w/v, about 0.25% w/v to about 0.30% w/v, about 0.30% w/v to about 2.0% w/v, about 0.30% w/v to about 1.9% w/v, about 0.30% w/v to about 1.8% w/v, about 0.30% w/v to about 1.7% w/v, about 0.30% w/v to about 1.6% w/v, about 0.30% w/v to about 1.5% w/v, about 0.30% w/v to about 1.4% w/v, about 0.30% w/v to about 1.3% w/v, about 0.30% w/v to about 1.2% w/v, about 0.30% w/v to about 1.1% w/v, about 0.30% w/v to about 1.0% w/v, about 0.30% w/v to about 0.90% w/v, about 0.30% w/v to about 0.80% w/v, about 0.30% w/v to about 0.75% w/v, about 0.30% w/v to about 0.70% w/v, about 0.30% w/v to about 0.65% w/v, about 0.30% w/v to about 0.60% w/v, about 0.30% w/v to about 0.55% w/v, about 0.30% w/v to about 0.50% w/v, about 0.30% w/v to about 0.45% w/v, about 0.30% w/v to about 0.40% w/v, about 0.30% w/v to about 0.35% w/v, about 0.35% w/v to about 2.0% w/v, about 0.35% w/v to about 1.9% w/v, about 0.35% w/v to about 1.8% w/v, about 0.35% w/v to about 1.7% w/v, about 0.35% w/v to about 1.6% w/v, about 0.35% w/v to about 1.5% w/v, about 0.35% w/v to about 1.4% w/v, about 0.35% w/v to about 1.3% w/v, about 0.35% w/v to about 1.2% w/v, about 0.35% w/v to about 1.1% w/v, about 0.35% w/v to about 1.0% w/v, about 0.35% w/v to about 0.90% w/v, about 0.35% w/v to about 0.80% w/v, about 0.35% w/v to about 0.75% w/v, about 0.35% w/v to about 0.70% w/v, about 0.35% w/v to about 0.65% w/v, about 0.35% w/v to about 0.60% w/v, about 0.35% w/v to about 0.55% w/v, about 0.35% w/v to about 0.50% w/v, about 0.35% w/v to about 0.45% w/v, about 0.35% w/v to about 0.40% w/v, about 0.40% w/v to about 2.0% w/v, about 0.40% w/v to about 1.9% w/v, about 0.40% w/v to about 1.8% w/v, about 0.40% w/v to about 1.7% w/v, about 0.40% w/v to about 1.6% w/v, about 0.40% w/v to about 1.5% w/v, about 0.40% w/v to about 1.4% w/v, about 0.40% w/v to about 1.3% w/v, about 0.40% w/v to about 1.2% w/v, about 0.40% w/v to about 1.1% w/v, about 0.40% w/v to about 1.0% w/v, about 0.40% w/v to about 0.90% w/v, about 0.40% w/v to about 0.80% w/v, about 0.40% w/v to about 0.75% w/v, about 0.40% w/v to about 0.70% w/v, about 0.40% w/v to about 0.65% w/v, about 0.40% w/v to about 0.60% w/v, about 0.40% w/v to about 0.55% w/v, about 0.40% w/v to about 0.50% w/v, about 0.40% w/v to about 0.45% w/v, about 0.45% w/v to about 2.0% w/v, about 0.45% w/v to about 1.9% w/v, about 0.45% w/v to about 1.8% w/v, about 0.45% w/v to about 1.7% w/v, about 0.45% w/v to about 1.6% w/v, about 0.45% w/v to about 1.5% w/v, about 0.45% w/v to about 1.4% w/v, about 0.45% w/v to about 1.3% w/v, about 0.45% w/v to about 1.2% w/v, about 0.45% w/v to about 1.1% w/v, about 0.45% w/v to about 1.0% w/v, about 0.45% w/v to about 0.90% w/v, about 0.45% w/v to about 0.80% w/v, about 0.45% w/v to about 0.75% w/v, about 0.45% w/v to about 0.70% w/v, about 0.45% w/v to about 0.65% w/v, about 0.45% w/v to about 0.60% w/v, about 0.45% w/v to about 0.55% w/v, about 0.45% w/v to about 0.50% w/v, about 0.50% w/v to about 2.0% w/v, about 0.50% w/v to about 1.9% w/v, about 0.50% w/v to about 1.8% w/v, about 0.50% w/v to about 1.7% w/v, about 0.50% w/v to about 1.6% w/v, about 0.50% w/v to about 1.5% w/v, about 0.50% w/v to about 1.4% w/v, about 0.50% w/v to about 1.3% w/v, about 0.50% w/v to about 1.2% w/v, about 0.50% w/v to about 1.1% w/v, about 0.50% w/v to about 1.0% w/v, about 0.50% w/v to about 0.90% w/v, about 0.50% w/v to about 0.80% w/v, about 0.50% w/v to about 0.75% w/v, about 0.50% w/v to about 0.70% w/v, about 0.50% w/v to about 0.65% w/v, about 0.50% w/v to about 0.60% w/v, about 0.50% w/v to about 0.55% w/v, about 0.55% w/v to about 2.0% w/v, about 0.55% w/v to about 1.9% w/v, about 0.55% w/v to about 1.8% w/v, about 0.55% w/v to about 1.7% w/v, about 0.55% w/v to about 1.6% w/v, about 0.55% w/v to about 1.5% w/v, about 0.55% w/v to about 1.4% w/v, about 0.55% w/v to about 1.3% w/v, about 0.55% w/v to about 1.2% w/v, about 0.55% w/v to about 1.1% w/v, about 0.55% w/v to about 1.0% w/v, about 0.55% w/v to about 0.90% w/v, about 0.55% w/v to about 0.80% w/v, about 0.55% w/v to about 0.75% w/v, about 0.55% w/v to about 0.70% w/v, about 0.55% w/v to about 0.65% w/v, about 0.55% w/v to about 0.60% w/v, about 0.60% w/v to about 2.0% w/v, about 0.60% w/v to about 1.9% w/v, about 0.60% w/v to about 1.8% w/v, about 0.60% w/v to about 1.7% w/v, about 0.60% w/v to about 1.6% w/v, about 0.60% w/v to about 1.5% w/v, about 0.60% w/v to about 1.4% w/v, about 0.60% w/v to about 1.3% w/v, about 0.60% w/v to about 1.2% w/v, about 0.60% w/v to about 1.1% w/v, about 0.60% w/v to about 1.0% w/v, about 0.60% w/v to about 0.90% w/v, about 0.60% w/v to about 0.80% w/v, about 0.60% w/v to about 0.75% w/v, about 0.60% w/v to about 0.70% w/v, about 0.60% w/v to about 0.65% w/v, about 0.65% w/v to about 2.0% w/v, about 0.65% w/v to about 1.9% w/v, about 0.65% w/v to about 1.8% w/v, about 0.65% w/v to about 1.7% w/v, about 0.65% w/v to about 1.6% w/v, about 0.65% w/v to about 1.5% w/v, about 0.65% w/v to about 1.4% w/v, about 0.65% w/v to about 1.3% w/v, about 0.65% w/v to about 1.2% w/v, about 0.65% w/v to about 1.1% w/v, about 0.65% w/v to about 1.0% w/v, about 0.65% w/v to about 0.90% w/v, about 0.65% w/v to about 0.80% w/v, about 0.65% w/v to about 0.75% w/v, about 0.65% w/v to about 0.70% w/v, about 0.70% w/v to about 2.0% w/v, about 0.70% w/v to about 1.9% w/v, about 0.70% w/v to about 1.8% w/v, about 0.70% w/v to about 1.7% w/v, about 0.70% w/v to about 1.6% w/v, about 0.70% w/v to about 1.5% w/v, about 0.70% w/v to about 1.4% w/v, about 0.70% w/v to about 1.3% w/v, about 0.70% w/v to about 1.2% w/v, about 0.70% w/v to about 1.1% w/v, about 0.70% w/v to about 1.0% w/v, about 0.70% w/v to about 0.90% w/v, about 0.70% w/v to about 0.80% w/v, about 0.70% w/v to about 0.75% w/v, about 0.75% w/v to about 2.0% w/v, about 0.75% w/v to about 1.9% w/v, about 0.75% w/v to about 1.8% w/v, about 0.75% w/v to about 1.7% w/v, about 0.75% w/v to about 1.6% w/v, about 0.75% w/v to about 1.5% w/v, about 0.75% w/v to about 1.4% w/v, about 0.75% w/v to about 1.3% w/v, about 0.75% w/v to about 1.2% w/v, about 0.75% w/v to about 1.1% w/v, about 0.75% w/v to about 1.0% w/v, about 0.75% w/v to about 0.90% w/v, about 0.75% w/v to about 0.80% w/v, about 0.80% w/v to about 2.0% w/v, about 0.80% w/v to about 1.9% w/v, about 0.80% w/v to about 1.8% w/v, about 0.80% w/v to about 1.7% w/v, about 0.80% w/v to about 1.6% w/v, about 0.80% w/v to about 1.5% w/v, about 0.80% w/v to about 1.4% w/v, about 0.80% w/v to about 1.3% w/v, about 0.80% w/v to about 1.2% w/v, about 0.80% w/v to about 1.1% w/v, about 0.80% w/v to about 1.0% w/v, about 0.80% w/v to about 0.90% w/v, about 0.90% w/v to about 2.0% w/v, about 0.90% w/v to about 1.9% w/v, about 0.90% w/v to about 1.8% w/v, about 0.90% w/v to about 1.7% w/v, about 0.90% w/v to about 1.6% w/v, about 0.90% w/v to about 1.5% w/v, about 0.90% w/v to about 1.4% w/v, about 0.90% w/v to about 1.3% w/v, about 0.90% w/v to about 1.2% w/v, about 0.90% w/v to about 1.1% w/v, about 0.90% w/v to about 1.0% w/v, about 1.0% w/v to about 2.0% w/v, about 1.0% w/v to about 1.9% w/v, about 1.0% w/v to about 1.8% w/v, about 1.0% w/v to about 1.7% w/v, about 1.0% w/v to about 1.6% w/v, about 1.0% w/v to about 1.5% w/v, about 1.0% w/v to about 1.4% w/v, about 1.0% w/v to about 1.3% w/v, about 1.0% w/v to about 1.2% w/v, about 1.0% w/v to about 1.1% w/v, about 1.1% w/v to about 2.0% w/v, about 1.1% w/v to about 1.9% w/v, about 1.1% w/v to about 1.8% w/v, about 1.1% w/v to about 1.7% w/v, about 1.1% w/v to about 1.6% w/v, about 1.1% w/v to about 1.5% w/v, about 1.1% w/v to about 1.4% w/v, about 1.1% w/v to about 1.3% w/v, about 1.1% w/v to about 1.2% w/v, about 1.2% w/v to about 2.0% w/v, about 1.2% w/v to about 1.9% w/v, about 1.2% w/v to about 1.8% w/v, about 1.2% w/v to about 1.7% w/v, about 1.2% w/v to about 1.6% w/v, about 1.2% w/v to about 1.5% w/v, about 1.2% w/v to about 1.4% w/v, about 1.2% w/v to about 1.3% w/v, about 1.3% w/v to about 2.0% w/v, about 1.3% w/v to about 1.9% w/v, about 1.3% w/v to about 1.8% w/v, about 1.3% w/v to about 1.7% w/v, about 1.3% w/v to about 1.6% w/v, about 1.3% w/v to about 1.5% w/v, about 1.3% w/v to about 1.4% w/v, about 1.4% w/v to about 2.0% w/v, about 1.4% w/v to about 1.9% w/v, about 1.4% w/v to about 1.8% w/v, about 1.4% w/v to about 1.7% w/v, about 1.4% w/v to about 1.6% w/v, about 1.4% w/v to about 1.5% w/v, about 1.5% w/v to about 2.0% w/v, about 1.5% w/v to about 1.9% w/v, about 1.5% w/v to about 1.8% w/v, about 1.5% w/v to about 1.7% w/v, about 1.5% w/v to about 1.6% w/v, about 1.6% w/v to about 2.0% w/v, about 1.6% w/v to about 1.9% w/v, about 1.6% w/v to about 1.8% w/v, about 1.6% w/v to about 1.7% w/v, about 1.7% w/v to about 2.0% w/v, about 1.7% w/v to about 1.9% w/v, about 1.7% w/v to about 1.8% w/v, about 1.8% w/v to about 2.0% w/v, about 1.8% w/v to about 1.9% w/v, or about 1.9% w/v to about 2.0% w/v).

pH

In some embodiments, the pharmaceutically acceptable aqueous buffered solutions described herein have a pH of e.g. about 6.0 to about 9.0, about 6.0 to about 8.8, about 6.0 to about 8.6, about 6.0 to about 8.4, about 6.0 to about 8.2, about 6.0 to about 8.0, about 6.0 to about 7.8, about 6.0 to about 7.6, about 6.0 to about 7.4, about 6.0 to about 7.4, about 6.0 to about 7.2, about 6.0 to about 7.0, about 6.0 to about 6.8, about 6.0 to about 6.6, about 6.0 to about 6.4, about 6.0 to about 6.2, about 6.2 to about 9.0, about 6.2 to about 8.8, about 6.2 to about 8.6, about 6.2 to about 8.4, about 6.2 to about 8.2, about 6.2 to about 8.0, about 6.2 to about 7.8, about 6.2 to about 7.6, about 6.2 to about 7.4, about 6.2 to about 7.4, about 6.2 to about 7.2, about 6.2 to about 7.0, about 6.2 to about 6.8, about 6.2 to about 6.6, about 6.2 to about 6.4, about 6.4 to about 9.0, about 6.4 to about 8.8, about 6.4 to about 8.6, about 6.4 to about 8.4, about 6.4 to about 8.2, about 6.4 to about 8.0, about 6.4 to about 7.8, about 6.4 to about 7.6, about 6.4 to about 7.4, about 6.4 to about 7.4, about 6.4 to about 7.2, about 6.4 to about 7.0, about 6.4 to about 6.8, about 6.4 to about 6.6, about 6.6 to about 9.0, about 6.6 to about 8.8, about 6.6 to about 8.6, about 6.6 to about 8.4, about 6.6 to about 8.2, about 6.6 to about 8.0, about 6.6 to about 7.8, about 6.6 to about 7.6, about 6.6 to about 7.4, about 6.6 to about 7.4, about 6.6 to about 7.2, about 6.6 to about 7.0, about 6.6 to about 6.8, about 6.8 to about 9.0, about 6.8 to about 8.8, about 6.8 to about 8.6, about 6.8 to about 8.4, about 6.8 to about 8.2, about 6.8 to about 8.0, about 6.8 to about 7.8, about 6.8 to about 7.6, about 6.8 to about 7.4, about 6.8 to about 7.4, about 6.8 to about 7.2, about 6.8 to about 7.0, about 7.0 to about 9.0, about 7.0 to about 8.8, about 7.0 to about 8.6, about 7.0 to about 8.4, about 7.0 to about 8.2, about 7.0 to about 8.0, about 7.0 to about 7.8, about 7.0 to about 7.6, about 7.0 to about 7.4, about 7.0 to about 7.4, about 7.0 to about 7.2, about 7.2 to about 9.0, about 7.2 to about 8.8, about 7.2 to about 8.6, about 7.2 to about 8.4, about 7.2 to about 8.2, about 7.2 to about 8.0, about 7.2 to about 7.8, about 7.2 to about 7.6, about 7.2 to about 7.4, about 7.4 to about 9.0, about 7.4 to about 8.8, about 7.4 to about 8.6, about 7.4 to about 8.4, about 7.4 to about 8.2, about 7.4 to about 8.0, about 7.4 to about 7.8, about 7.4 to about 7.6, about 7.6 to about 9.0, about 7.6 to about 8.8, about 7.6 to about 8.6, about 7.6 to about 8.4, about 7.6 to about 8.2, about 7.6 to about 8.0, about 7.6 to about 7.8, about 7.8 to about 9.0, about 7.8 to about 8.8, about 7.8 to about 8.6, about 7.8 to about 8.4, about 7.8 to about 8.2, about 7.8 to about 8.0, about 8.0 to about 9.0, about 8.0 to about 8.8, about 8.0 to about 8.6, about 8.0 to about 8.4, about 8.0 to about 8.2, about 8.2 to about 9.0, about 8.2 to about 8.8, about 8.2 to about 8.6, about 8.2 to about 8.4, about 8.4 to about 9.0, about 8.4 to about 8.8, about 8.4 to about 8.6, about 8.6 to about 9.0, about 8.6 to about 8.8, or about 8.8 to about 9.0.

### Osmolarity

In some embodiments, the pharmaceutically acceptable aqueous buffered solutions described herein have an osmolarity of about 100 mOsm/L to about 400 mOsm/L (e.g., about 100 mOsm/L to about 380 mOsm/L, about 100 mOsm/L to about 360 mOsm/L, about 100 mOsm/L to about 340 mOsm/L, about 100 mOsm/L to about 320 mOsm/L, about 100 mOsm/L to about 300 mOsm/L, about 100 mOsm/L to about 280 mOsm/L, about 100 mOsm/L to about 260 mOsm/L, about 100 mOsm/L to about 250 mOsm/L, about 100 mOsm/L to about 200 mOsm/L, about 100 mOsm/L to about 150 mOsm/L, about 150 mOsm/L to about 400 mOsm/L, about 150 mOsm/L to about 380 mOsm/L, about 150 mOsm/L to about 360 mOsm/L, about 150 mOsm/L to about 340 mOsm/L, about 150 mOsm/L to about 320 mOsm/L, about 150 mOsm/L to about 300 mOsm/L, about 150 mOsm/L to about 280 mOsm/L, about 150 mOsm/L to about 260 mOsm/L, about 150 mOsm/L to about 250 mOsm/L, about 150 mOsm/L to about 200 mOsm/L, about 200 mOsm/L to about 400 mOsm/L, about 200 mOsm/L to about 380 mOsm/L, about 200 mOsm/L to about 360 mOsm/L, about 200 mOsm/L to about 340 mOsm/L, about 200 mOsm/L to about 320 mOsm/L, about 200 mOsm/L to about 300 mOsm/L, about 200 mOsm/L to about 280 mOsm/L, about 200 mOsm/L to about 260 mOsm/L, about 200 mOsm/L to about 250 mOsm/L, about 250 mOsm/L to about 400 mOsm/L, about 250 mOsm/L to about 380 mOsm/L, about 250 mOsm/L to about 360 mOsm/L, about 250 mOsm/L to about 340 mOsm/L, about 250 mOsm/L to about 320 mOsm/L, about 250 mOsm/L to about 300 mOsm/L, about 250 mOsm/L to about 280 mOsm/L, about 250 mOsm/L to about 260 mOsm/L, about 260 mOsm/L to about 400 mOsm/L, about 260 mOsm/L to about 380 mOsm/L, about 260 mOsm/L to about 360 mOsm/L, about 260 mOsm/L to about 340 mOsm/L, about 260 mOsm/L to about 320 mOsm/L, about 260 mOsm/L to about 300 mOsm/L, about 260 mOsm/L to about 280 mOsm/L, about 280 mOsm/L to about 400 mOsm/L, about 280 mOsm/L to about 380 mOsm/L, about 280 mOsm/L to about 360 mOsm/L, about 280 mOsm/L to about 340 mOsm/L, about 280 mOsm/L to about 320 mOsm/L, about 280 mOsm/L to about 300 mOsm/L, about 300 mOsm/L to about 400 mOsm/L, about 300 mOsm/L to about 380 mOsm/L, about 300 mOsm/L to about 360 mOsm/L, about 300 mOsm/L to about 340 mOsm/L, about 300 mOsm/L to about 320 mOsm/L, about 320 mOsm/L to about 400 mOsm/L, about 320 mOsm/L to about 380 mOsm/L, about 320 mOsm/L to about 360 mOsm/L, about 320 mOsm/L to about 340 mOsm/L, about 340 mOsm/L to about 400 mOsm/L, about 340 mOsm/L to about 380 mOsm/L, about 340 mOsm/L to about 360 mOsm/L, about 360 mOsm/L to about 400 mOsm/L, about 360 mOsm/L to about 380 mOsm/L, or about 380 mOsm/L to about 400 mOsm/L).

### Exemplary Embodiments

In some embodiments, the compositions provided herein include a pharmaceutically acceptable aqueous buffered solution comprising: about 70 mM to about 90 mM (e.g., about 75 mM to about 85 mM, or about 80 mM) of sodium ion, about 30 mM to about 50 mM (e.g., about 37 mM to about 47 mM, or about 42 mM) of potassium ion; about 0.01 mM to about 0.15 mM (e.g., about 0.01 mM to about 0.10 mM, or about 0.05 mM) of calcium ion; about 1 mM to about 10 mM (e.g., about 4 mM to about 6 mM, or about 5 mM) of magnesium ion; about 5 mM to about 10 mM (e.g., about 8 mM to about 10 mM, or about 10.1 mM) of chloride ion; about 5 mM to about 15 mM (e.g., about 8 mM to about 12 mM, or about 10 mM) of phosphate ion; about 1 mM to about 10 mM (e.g., about 3 mM to about 7 mM, or about 5 mM) of bicarbonate ion; about 15 mM to about 35 mM (e.g., about 20 mM to about 30 mM, or about 25 mM) of a buffer (e.g., HEPES); about 80 mM to about 120 mM (e.g., about 90 mM to about 110 mM, or about 100 mM) of lactobionate; about 30 mM to about 50 mM (e.g., about 35 mM to about 45 mM, or about 40 mM) of mannitol; about 0.1 mM to about 3 mM (e.g., about 1.5 mM to about 2.5 mM, or about 2.0 mM) of adenosine; and about 0.1 mM to about 2 mM (e.g., about 0.5 mM to about 1.5 mM, or about 1.0 mM) of adenine; and having a pH of about 7.4 to about 7.8 (e.g., about 7.5 to about 7.6, or about 7.57).

In some embodiments, the compositions provided herein include an engineered enucleated erythroid cell (or a population thereof) comprising one or more exogenous proteins present on their membrane (e.g., a combination of: a first exogenous protein comprising 4-1BBL, or a fragment thereof, linked to a transmembrane protein (e.g., GPA, or a transmembrane fragment thereof); and a second exogenous protein comprising IL-15, or a fragment thereof, linked to an extracellular portion of IL-15 receptor alpha (IL-15Rα), or a fragment thereof (e.g., an IL-15Rα sushi-binding domain), linked to a transmembrane protein (e.g., GPA, or a transmembrane fragment thereof), e.g., as described in U.S. Patent Application Publication No. 2019/0298769, incorporated herein by reference) and a pharmaceutically acceptable aqueous buffered solution comprising: about 70 mM to about 90 mM (e.g., about 75 mM to about 85 mM, or about 80 mM) of sodium ion, about 30 mM to about 50 mM (e.g., about 37 mM to about 47 mM, or about 42 mM) of potassium ion; about 0.01 mM to about 0.15 mM (e.g., about 0.01 mM to about 0.10 mM, or about 0.05 mM) of calcium ion; about 1 mM to about 10 mM (e.g., about 4 mM to about 6 mM, or about 5 mM) of magnesium ion; about 5 mM to about 10 mM (e.g., about 8 mM to about 10 mM, or about 10.1 mM) of chloride ion; about 5 mM to about 15 mM (e.g., about 8 mM to about 12 mM, or about 10 mM) of phosphate ion; about 1 mM to about 10 mM (e.g., about 3 mM to about 7 mM, or about 5 mM) of bicarbonate ion; about 15 mM to about 35 mM (e.g., about 20 mM to about 30 mM, or about 25 mM) of a buffer (e.g., HEPES); about 80 mM to about 120 mM (e.g., about 90 mM to about 110 mM, or about 100 mM) of lactobionate; about 30 mM to about 50 mM (e.g., about 35 mM to about 45 mM, or about 40 mM) of mannitol; about 0.1 mM to about 3 mM (e.g., about 1.5 mM to about 2.5 mM, or about 2.0 mM) of adenosine; and about 0.1 mM to about 2 mM (e.g., about 0.5 mM to about 1.5 mM, or about 1.0 mM) of adenine; and having a pH of about 7.4 to about 7.8 (e.g., about 7.5 to about 7.6, or about 7.57).

In some embodiments, the compositions provided herein include an engineered enucleated erythroid cell (or a population thereof) comprising one or more exogenous proteins present on their membrane (e.g., a combination of: a first exogenous protein comprising 4-1BBL, or a fragment thereof, linked to a transmembrane protein (e.g., GPA, or a transmembrane fragment thereof); and a second exogenous protein comprising IL-12 p40, or a fragment thereof, linked to IL-12 p35, or a fragment thereof, linked to a transmembrane protein (e.g., GPA, or a transmembrane fragment thereof, or SMIM1, or a transmembrane fragment thereof), e.g., as described in U.S. Patent Application Publication No. 2019/0298769, incorporated herein by reference) and a pharmaceutically acceptable aqueous buffered solution comprising: about 70 mM to about 90 mM (e.g., about 75 mM to about 85 mM, or about 80 mM) of sodium ion, about 30 mM to about 50 mM (e.g., about 37 mM to about 47 mM, or about 42 mM) of potassium ion; about 0.01 mM to about 0.15 mM (e.g., about 0.01 mM to about 0.10 mM, or about 0.05 mM) of calcium ion; about 1 mM to about 10 mM (e.g., about 4 mM to about 6 mM, or about 5 mM) of magnesium ion; about 5 mM to about 10 mM (e.g., about 8 mM to about 10 mM, or about 10.1 mM) of chloride ion; about 5 mM to about 15 mM (e.g., about 8 mM to about 12 mM, or about 10 mM) of phosphate ion; about 1 mM to about 10 mM (e.g., about 3 mM to about 7 mM, or about 5 mM) of bicarbonate ion; about 15 mM to about 35 mM (e.g., about 20 mM to about 30 mM, or about 25 mM) of a buffer (e.g., HEPES); about 80 mM to about 120 mM (e.g., about 90 mM to about 110 mM, or about 100 mM) of lactobionate; about 30 mM to about 50 mM (e.g., about 35 mM to about 45 mM, or about 40 mM) of mannitol; about 0.1 mM to about 3 mM (e.g., about 1.5 mM to about 2.5 mM, or about 2.0 mM) of adenosine; and about 0.1 mM to about 2 mM (e.g., about 0.5 mM to about 1.5 mM, or about 1.0 mM) of adenine; and having a pH of about 7.4 to about 7.8 (e.g., about 7.5 to about 7.6, or about 7.57).

In some embodiments, the compositions provided herein include an engineered enucleated erythroid cell (or a population thereof) comprising one or more exogenous proteins present on their membrane (e.g., a combination of: a first exogenous protein comprising an antigenic peptide (e.g., an HPV antigen such as HPV16 E7₁₁₋₁₉) linked to beta 2 microglobulin (B2M), or a fragment thereof, linked to one or more of the alpha1, alpha2, and alpha 3 domains of an MHC class I protein (e.g., HLA*02:01), or fragment or variant thereof, linked to a transmembrane protein (e.g., GPA, or a transmembrane fragment thereof); a second exogenous protein comprising 4-1BBL, or a fragment thereof, linked to a transmembrane protein (e.g., GPA, or a transmembrane fragment thereof); and a second exogenous protein comprising IL-12 p40, or a fragment thereof, linked to IL-12 p35, or a fragment thereof, linked to a transmembrane protein (e.g., GPA, or a transmembrane fragment thereof, or SMIM1, or a transmembrane fragment thereof), e.g., as described in U.S. Patent Application Publication No. 2019/0290686, incorporated herein by reference) and a pharmaceutically acceptable aqueous buffered solution comprising: about 70 mM to about 90 mM (e.g., about 75 mM to about 85 mM, or about 80 mM) of sodium ion, about 30 mM to about 50 mM (e.g., about 37 mM to about 47 mM, or about 42 mM) of potassium ion; about 0.01 mM to about 0.15 mM (e.g., about 0.01 mM to about 0.10 mM, or about 0.05 mM) of calcium ion; about 1 mM to about 10 mM (e.g., about 4 mM to about 6 mM, or about 5 mM) of magnesium ion; about 5 mM to about 10 mM (e.g., about 8 mM to about 10 mM, or about 10.1 mM) of chloride ion; about 5 mM to about 15 mM (e.g., about 8 mM to about 12 mM, or about 10 mM) of phosphate ion; about 1 mM to about 10 mM (e.g., about 3 mM to about 7 mM, or about 5 mM) of bicarbonate ion; about 15 mM to about 35 mM (e.g., about 20 mM to about 30 mM, or about 25 mM) of a buffer (e.g., HEPES); about 80 mM to about 120 mM (e.g., about 90 mM to about 110 mM, or about 100 mM) of lactobionate; about 30 mM to about 50 mM (e.g., about 35 mM to about 45 mM, or about 40 mM) of mannitol; about 0.1 mM to about 3 mM (e.g., about 1.5 mM to about 2.5 mM, or about 2.0 mM) of adenosine; and about 0.1 mM to about 2 mM (e.g., about 0.5 mM to about 1.5 mM, or about 1.0 mM) of adenine; and having a pH of about 7.4 to about 7.8 (e.g., about 7.5 to about 7.6, or about 7.57).

In some embodiments, the compositions provided herein include a pharmaceutically acceptable aqueous buffered solution comprising: about 70 mM to about 90 mM (e.g., about 75 mM to about 85 mM, or about 80 mM) of sodium ion, about 30 mM to about 50 mM (e.g., about 37 mM to about 47 mM, or about 42 mM) of potassium ion; about 0.01 mM to about 0.15 mM (e.g., about 0.01 mM to about 0.10 mM, or about 0.05 mM) of calcium ion; about 1 mM to about 10 mM (e.g., about 4 mM to about 6 mM, or about 5 mM) of magnesium ion; about 5 mM to about 10 mM (e.g., about 8 mM to about 10 mM, or about 10.1 mM) of chloride ion; about 5 mM to about 15 mM (e.g., about 8 mM to about 12 mM, or about 10 mM) of phosphate ion; about 1 mM to about 10 mM (e.g., about 3 mM to about 7 mM, or about 5 mM) of bicarbonate ion; about 15 mM to about 35 mM (e.g., about 20 mM to about 30 mM, or about 25 mM) of a buffer (e.g., HEPES); about 80 mM to about 120 mM (e.g., about 90 mM to about 110 mM, or about 100 mM) of lactobionate; about 30 mM to about 50 mM (e.g., about 35 mM to about 45 mM, or about 40 mM) of mannitol; about 0.1 mM to about 3 mM (e.g., about 1.5 mM to about 2.5 mM, or about 2.0 mM) of adenosine; about 0.1 mM to about 2 mM (e.g., about 0.5 mM to about 1.5 mM, or about 1.0 mM) of adenine; and about 0.1% w/v to about 0.9% w/v (e.g., about 0.3% w/v to about 0.7% w/v, or about 0.5% w/v) of a poloxamer (e.g., poloxamer-188), and having a pH of about 7.4 to about 7.8 (e.g., about 7.5 to about 7.6, or about 7.57).

In some embodiments, the compositions provided herein include an engineered enucleated erythroid cell (or a population thereof) comprising one or more exogenous proteins present on their membrane (e.g., a combination of: a first exogenous protein comprising 4-1BBL, or a fragment thereof, linked to a transmembrane protein (e.g., GPA, or a transmembrane fragment thereof); and a second exogenous protein comprising IL-15, or a fragment thereof, linked to an extracellular portion of IL-15 receptor alpha (IL-15Rα), or a fragment thereof (e.g., an IL-15Rα sushi-binding domain), linked to a transmembrane protein (e.g., GPA, or a transmembrane fragment thereof), e.g., as described in U.S. Patent Application Publication No. 2019/0298769, incorporated herein by reference) and a pharmaceutically acceptable aqueous buffered solution comprising: about 70 mM to about 90 mM (e.g., about 75 mM to about 85 mM, or about 80 mM) of sodium ion, about 30 mM to about 50 mM (e.g., about 37 mM to about 47 mM, or about 42 mM) of potassium ion; about 0.01 mM to about 0.15 mM (e.g., about 0.01 mM to about 0.10 mM, or about 0.05 mM) of calcium ion; about 1 mM to about 10 mM (e.g., about 4 mM to about 6 mM, or about 5 mM) of magnesium ion; about 5 mM to about 10 mM (e.g., about 8 mM to about 10 mM, or about 10.1 mM) of chloride ion; about 5 mM to about 15 mM (e.g., about 8 mM to about 12 mM, or about 10 mM) of phosphate ion; about 1 mM to about 10 mM (e.g., about 3 mM to about 7 mM, or about 5 mM) of bicarbonate ion; about 15 mM to about 35 mM (e.g., about 20 mM to about 30 mM, or about 25 mM) of a buffer (e.g., HEPES); about 80 mM to about 120 mM (e.g., about 90 mM to about 110 mM, or about 100 mM) of lactobionate; about 30 mM to about 50 mM (e.g., about 35 mM to about 45 mM, or about 40 mM) of mannitol; about 0.1 mM to about 3 mM (e.g., about 1.5 mM to about 2.5 mM, or about 2.0 mM) of adenosine; about 0.1 mM to about 2 mM (e.g., about 0.5 mM to about 1.5 mM, or about 1.0 mM) of adenine; and about 0.1% w/v to about 0.9% w/v (e.g., about 0.3% w/v to about 0.7% w/v, or about 0.5% w/v) of a poloxamer (e.g., poloxamer-188), and having a pH of about 7.4 to about 7.8 (e.g., about 7.5 to about 7.6, or about 7.57).

In some embodiments, the compositions provided herein include an engineered enucleated erythroid cell (or a population thereof) comprising one or more exogenous proteins present on their membrane (e.g., a combination of: a first exogenous protein comprising 4-1BBL, or a fragment thereof, linked to a transmembrane protein (e.g., GPA, or a transmembrane fragment thereof); and a second exogenous protein comprising IL-12 p40, or a fragment thereof, linked to IL-12 p35, or a fragment thereof, linked to a transmembrane protein (e.g., GPA, or a transmembrane fragment thereof, or SMIM1, or a transmembrane fragment thereof), e.g., as described in U.S. Patent Application Publication No. 2019/0298769, incorporated herein by reference) and a pharmaceutically acceptable aqueous buffered solution comprising: about 70 mM to about 90 mM (e.g., about 75 mM to about 85 mM, or about 80 mM) of sodium ion, about 30 mM to about 50 mM (e.g., about 37 mM to about 47 mM, or about 42 mM) of potassium ion; about 0.01 mM to about 0.15 mM (e.g., about 0.01 mM to about 0.10 mM, or about 0.05 mM) of calcium ion; about 1 mM to about 10 mM (e.g., about 4 mM to about 6 mM, or about 5 mM) of magnesium ion; about 5 mM to about 10 mM (e.g., about 8 mM to about 10 mM, or about 10.1 mM) of chloride ion; about 5 mM to about 15 mM (e.g., about 8 mM to about 12 mM, or about 10 mM) of phosphate ion; about 1 mM to about 10 mM (e.g., about 3 mM to about 7 mM, or about 5 mM) of bicarbonate ion; about 15 mM to about 35 mM (e.g., about 20 mM to about 30 mM, or about 25 mM) of a buffer (e.g., HEPES); about 80 mM to about 120 mM (e.g., about 90 mM to about 110 mM, or about 100 mM) of lactobionate; about 30 mM to about 50 mM (e.g., about 35 mM to about 45 mM, or about 40 mM) of mannitol; about 0.1 mM to about 3 mM (e.g., about 1.5 mM to about 2.5 mM, or about 2.0 mM) of adenosine; about 0.1 mM to about 2 mM (e.g., about 0.5 mM to about 1.5 mM, or about 1.0 mM) of adenine; and about 0.1% w/v to about 0.9% w/v (e.g., about 0.3% w/v to about 0.7% w/v, or about 0.5% w/v) of a poloxamer (e.g., poloxamer-188), and having a pH of about 7.4 to about 7.8 (e.g., about 7.5 to about 7.6, or about 7.57).

In some embodiments, the compositions provided herein include an engineered enucleated erythroid cell (or a population thereof) comprising one or more exogenous proteins present on their membrane (e.g., a combination of: a first exogenous protein comprising an antigenic peptide (e.g., an HPV antigen such as HPV16 E7₁₁₋₁₉) linked to B2M, or a fragment thereof, linked to one or more of the alpha1, alpha2, and alpha 3 domains of an MHC class I protein (e.g., HLA*02:01), or fragment or variant thereof, linked to a transmembrane protein (e.g., GPA, or a transmembrane fragment thereof); a second exogenous protein comprising 4-1BBL, or a fragment thereof, linked to a transmembrane protein (e.g., GPA, or a transmembrane fragment thereof); and a second exogenous protein comprising IL-12 p40, or a fragment thereof, linked to IL-12 p35, or a fragment thereof, linked to a transmembrane protein (e.g., GPA, or a transmembrane fragment thereof, or SMIM1, or a transmembrane fragment thereof), e.g., as described in U.S. Patent Application Publication No. 2019/0290686, incorporated herein by reference) and a pharmaceutically acceptable aqueous buffered solution comprising: about 70 mM to about 90 mM (e.g., about 75 mM to about 85 mM, or about 80 mM) of sodium ion, about 30 mM to about 50 mM (e.g., about 37 mM to about 47 mM, or about 42 mM) of potassium ion; about 0.01 mM to about 0.15 mM (e.g., about 0.01 mM to about 0.10 mM, or about 0.05 mM) of calcium ion; about 1 mM to about 10 mM (e.g., about 4 mM to about 6 mM, or about 5 mM) of magnesium ion; about 5 mM to about 10 mM (e.g., about 8 mM to about 10 mM, or about 10.1 mM) of chloride ion; about 5 mM to about 15 mM (e.g., about 8 mM to about 12 mM, or about 10 mM) of phosphate ion; about 1 mM to about 10 mM (e.g., about 3 mM to about 7 mM, or about 5 mM) of bicarbonate ion; about 15 mM to about 35 mM (e.g., about 20 mM to about 30 mM, or about 25 mM) of a buffer (e.g., HEPES); about 80 mM to about 120 mM (e.g., about 90 mM to about 110 mM, or about 100 mM) of lactobionate; about 30 mM to about 50 mM (e.g., about 35 mM to about 45 mM, or about 40 mM) of mannitol; about 0.1 mM to about 3 mM (e.g., about 1.5 mM to about 2.5 mM, or about 2.0 mM) of adenosine; about 0.1 mM to about 2 mM (e.g., about 0.5 mM to about 1.5 mM, or about 1.0 mM) of adenine; and about 0.1% w/v to about 0.9% w/v (e.g., about 0.3% w/v to about 0.7% w/v, or about 0.5% w/v) of a poloxamer (e.g., poloxamer-188), and having a pH of about 7.4 to about 7.8 (e.g., about 7.5 to about 7.6, or about 7.57).

In some embodiments, the compositions provided herein include a pharmaceutically acceptable aqueous buffered solution comprising: about 70 mM to about 90 mM (e.g., about 75 mM to about 85 mM, or about 79.4 mM) of sodium ion, about 30 mM to about 50 mM (e.g., about 37 mM to about 47 mM, or about 41.7 mM) of potassium ion; about 0.01 mM to about 0.15 mM (e.g., about 0.01 mM to about 0.10 mM, or about 0.05 mM) of calcium ion; about 1 mM to about 10 mM (e.g., about 4 mM to about 6 mM, or about 5 mM) of magnesium ion; about 5 mM to about 10 mM (e.g., about 8 mM to about 10 mM, or about 10.0 mM) of chloride ion; about 5 mM to about 15 mM (e.g., about 8 mM to about 12 mM, or about 9.9 mM) of phosphate ion; about 1 mM to about 10 mM (e.g., about 3 mM to about 7 mM, or about 5 mM) of bicarbonate ion; about 15 mM to about 35 mM (e.g., about 20 mM to about 30 mM, or about 24.8 mM) of a buffer (e.g., HEPES); about 80 mM to about 120 mM (e.g., about 90 mM to about 110 mM, or about 99.2 mM) of lactobionate; about 30 mM to about 50 mM (e.g., about 35 mM to about 45 mM, or about 39.7 mM) of mannitol; about 0.1 mM to about 3 mM (e.g., about 1.5 mM to about 2.5 mM, or about 2.0 mM) of adenosine; about 0.1 mM to about 2 mM (e.g., about 0.5 mM to about 1.5 mM, or about 1.0 mM) of adenine; and about 0.1% w/v to about 0.3% w/v (e.g., about 0.15% w/v to about 0.25% w/v, or about 0.20% w/v) of a serum albumin (e.g., human serum albumin), and having a pH of about 7.4 to about 7.8 (e.g., about 7.5 to about 7.6, or about 7.57). In some embodiments, the pharmaceutically acceptable aqueous buffered solution further comprises about 0.1% w/v to about 0.9% w/v (e.g., about 0.3% w/v to about 0.7% w/v, or about 0.5% w/v) of a poloxamer (e.g., poloxamer-188).

In some embodiments, the compositions provided herein include an engineered enucleated erythroid cell (or a population thereof) comprising one or more exogenous proteins present on their membrane (e.g., a combination of: a first exogenous protein comprising 4-1BBL, or a fragment thereof, linked to a transmembrane protein (e.g., GPA, or a transmembrane fragment thereof); and a second exogenous protein comprising IL-15, or a fragment thereof, linked to an extracellular portion of IL-15Rα, or a fragment thereof (e.g., an IL-15Rα sushi-binding domain), linked to a transmembrane protein (e.g., GPA, or a transmembrane fragment thereof), e.g., as described in U.S. Patent Application Publication No. 2019/0298769, incorporated herein by reference) and a pharmaceutically acceptable aqueous buffered solution comprising: about 70 mM to about 90 mM (e.g., about 75 mM to about 85 mM, or about 79.4 mM) of sodium ion, about 30 mM to about 50 mM (e.g., about 37 mM to about 47 mM, or about 41.7 mM) of potassium ion; about 0.01 mM to about 0.15 mM (e.g., about 0.01 mM to about 0.10 mM, or about 0.05 mM) of calcium ion; about 1 mM to about 10 mM (e.g., about 4 mM to about 6 mM, or about 5 mM) of magnesium ion; about 5 mM to about 10 mM (e.g., about 8 mM to about 10 mM, or about 10.0 mM) of chloride ion; about 5 mM to about 15 mM (e.g., about 8 mM to about 12 mM, or about 9.9 mM) of phosphate ion; about 1 mM to about 10 mM (e.g., about 3 mM to about 7 mM, or about 5 mM) of bicarbonate ion; about 15 mM to about 35 mM (e.g., about 20 mM to about 30 mM, or about 24.8 mM) of a buffer (e.g., HEPES); about 80 mM to about 120 mM (e.g., about 90 mM to about 110 mM, or about 99.2 mM) of lactobionate; about 30 mM to about 50 mM (e.g., about 35 mM to about 45 mM, or about 39.7 mM) of mannitol; about 0.1 mM to about 3 mM (e.g., about 1.5 mM to about 2.5 mM, or about 2.0 mM) of adenosine; about 0.1 mM to about 2 mM (e.g., about 0.5 mM to about 1.5 mM, or about 1.0 mM) of adenine; and about 0.1% w/v to about 0.3% w/v (e.g., about 0.15% w/v to about 0.25% w/v, or about 0.20% w/v) of a serum albumin (e.g., human serum albumin), and having a pH of about 7.4 to about 7.8 (e.g., about 7.5 to about 7.6, or about 7.57). In some embodiments, the pharmaceutically acceptable aqueous buffered solution further comprises about 0.1% w/v to about 0.9% w/v (e.g., about 0.3% w/v to about 0.7% w/v, or about 0.5% w/v) of a poloxamer (e.g., poloxamer-188).

In some embodiments, the compositions provided herein include an engineered enucleated erythroid cell (or a population thereof) comprising one or more exogenous proteins present on their membrane (e.g., a combination of: a first exogenous protein comprising 4-1BBL, or a fragment thereof, linked to a transmembrane protein (e.g., GPA, or a transmembrane fragment thereof); and a second exogenous protein comprising IL-12 p40, or a fragment thereof, linked to IL-12 p35, or a fragment thereof, linked to a transmembrane protein (e.g., GPA, or a transmembrane fragment thereof, or SMIM1, or a transmembrane fragment thereof), e.g., as described in U.S. Patent Application Publication No. 2019/0298769, incorporated herein by reference) and a pharmaceutically acceptable aqueous buffered solution comprising: about 70 mM to about 90 mM (e.g., about 75 mM to about 85 mM, or about 79.4 mM) of sodium ion, about 30 mM to about 50 mM (e.g., about 37 mM to about 47 mM, or about 41.7 mM) of potassium ion; about 0.01 mM to about 0.15 mM (e.g., about 0.01 mM to about 0.10 mM, or about 0.05 mM) of calcium ion; about 1 mM to about 10 mM (e.g., about 4 mM to about 6 mM, or about 5 mM) of magnesium ion; about 5 mM to about 10 mM (e.g., about 8 mM to about 10 mM, or about 10.0 mM) of chloride ion; about 5 mM to about 15 mM (e.g., about 8 mM to about 12 mM, or about 9.9 mM) of phosphate ion; about 1 mM to about 10 mM (e.g., about 3 mM to about 7 mM, or about 5 mM) of bicarbonate ion; about 15 mM to about 35 mM (e.g., about 20 mM to about 30 mM, or about 24.8 mM) of a buffer (e.g., HEPES); about 80 mM to about 120 mM (e.g., about 90 mM to about 110 mM, or about 99.2 mM) of lactobionate; about 30 mM to about 50 mM (e.g., about 35 mM to about 45 mM, or about 39.7 mM) of mannitol; about 0.1 mM to about 3 mM (e.g., about 1.5 mM to about 2.5 mM, or about 2.0 mM) of adenosine; about 0.1 mM to about 2 mM (e.g., about 0.5 mM to about 1.5 mM, or about 1.0 mM) of adenine; and about 0.1% w/v to about 0.3% w/v (e.g., about 0.15% w/v to about 0.25% w/v, or about 0.20% w/v) of a serum albumin (e.g., human serum albumin), and having a pH of about 7.4 to about 7.8 (e.g., about 7.5 to about 7.6, or about 7.57). In some embodiments, the pharmaceutically acceptable aqueous buffered solution further comprises about 0.1% w/v to about 0.9% w/v (e.g., about 0.3% w/v to about 0.7% w/v, or about 0.5% w/v) of a poloxamer (e.g., poloxamer-188).

In some embodiments, the compositions provided herein include an engineered enucleated erythroid cell comprising exogenous proteins present on their membrane (e.g., a combination of: a first exogenous protein comprising an antigenic peptide (e.g., an HPV antigen such as HPV16 E7₁₁₋₁₉) linked to B2M, or a fragment thereof, linked to one or more of the alpha1, alpha2, and alpha 3 domains of an MHC class I protein (e.g., HLA*02:01), or fragment or variant thereof, linked to a transmembrane protein (e.g., GPA, or a transmembrane fragment thereof); a second exogenous protein comprising 4-1BBL, or a fragment thereof, linked to a transmembrane protein (e.g., GPA, or a transmembrane fragment thereof); and a second exogenous protein comprising IL-12 p40, or a fragment thereof, linked to IL-12 p35, or a fragment thereof, linked to a transmembrane protein (e.g., GPA, or a transmembrane fragment thereof, or SMIM1, or a transmembrane fragment thereof), e.g., as described in U.S. Patent Application Publication No. 2019/0290686, incorporated herein by reference) and a pharmaceutically acceptable aqueous buffered solution comprising: about 70 mM to about 90 mM (e.g., about 75 mM to about 85 mM, or about 79.4 mM) of sodium ion, about 30 mM to about 50 mM (e.g., about 37 mM to about 47 mM, or about 41.7 mM) of potassium ion; about 0.01 mM to about 0.15 mM (e.g., about 0.01 mM to about 0.10 mM, or about 0.05 mM) of calcium ion; about 1 mM to about 10 mM (e.g., about 4 mM to about 6 mM, or about 5 mM) of magnesium ion; about 5 mM to about 10 mM (e.g., about 8 mM to about 10 mM, or about 10.0 mM) of chloride ion; about 5 mM to about 15 mM (e.g., about 8 mM to about 12 mM, or about 9.9 mM) of phosphate ion; about 1 mM to about 10 mM (e.g., about 3 mM to about 7 mM, or about 5 mM) of bicarbonate ion; about 15 mM to about 35 mM (e.g., about 20 mM to about 30 mM, or about 24.8 mM) of a buffer (e.g., HEPES); about 80 mM to about 120 mM (e.g., about 90 mM to about 110 mM, or about 99.2 mM) of lactobionate; about 30 mM to about 50 mM (e.g., about 35 mM to about 45 mM, or about 39.7 mM) of mannitol; about 0.1 mM to about 3 mM (e.g., about 1.5 mM to about 2.5 mM, or about 2.0 mM) of adenosine; about 0.1 mM to about 2 mM (e.g., about 0.5 mM to about 1.5 mM, or about 1.0 mM) of adenine; and about 0.1% w/v to about 0.3% w/v (e.g., about 0.15% w/v to about 0.25% w/v, or about 0.20% w/v) of a serum albumin (e.g., human serum albumin), and having a pH of about 7.4 to about 7.8 (e.g., about 7.5 to about 7.6, or about 7.57). In some embodiments, the pharmaceutically acceptable aqueous buffered solution further comprises about 0.1% w/v to about 0.9% w/v (e.g., about 0.3% w/v to about 0.7% w/v, or about 0.5% w/v) of a poloxamer (e.g., poloxamer-188).

### Kits

Also provided herein are kits that include any of the compositions provided herein. Also provided herein are kits that include one or more sterile vessels containing any of the compositions described herein (e.g., a sterile conical tube, a sterile petri dish, a sterile vial (e.g., a borosilicate glass vial), and sterile plastic bags (a di-2-ethylhexyl phthalate (DEHP)-plasticized polyvinyl chloride (PVC) bag, or n-butyryl-tri(n-hexyl)-citrate (BTHC)-plasticized PVC bag). In some embodiments, any of the kits provided herein can further include instructions for administration of any of the compositions to a subject in need thereof.

Some embodiments of the kits described herein include a suitable single dosage form of any of the compositions described herein. For example, a single dosage form of any of the compositions described herein can have a volume of, e.g., about 0.5 mL to about 2 L, about 0.5 mL to about 1800 mL, about 0.5 mL to about 1500 mL, about 0.5 mL to about 1200 mL, about 0.5 mL to about 1000 mL, about 0.5 mL to about 800 mL, about 0.5 mL to about 600 mL, about 0.5 mL to about 500 mL, about 0.5 mL to about 450 mL, about 0.5 mL to about 400 mL, about 0.5 mL to about 350 mL, about 0.5 mL to about 300 mL, about 0.5 mL to about 250 mL, about 0.5 mL to about 200 mL, about 0.5 mL to about 180 mL, about 0.5 mL to about 160 mL, about 0.5 mL to about 140 mL, about 0.5 mL to about 120 mL, about 0.5 mL to about 100 mL, about 0.5 mL to about 80 mL, about 0.5 mL to about 60 mL, about 0.5 mL to about 40 mL, about 0.5 mL to about 20 mL, about 0.5 mL to about 10 mL, about 0.5 mL to about 5 mL, about 0.5 mL to about 1.0 mL, about 1.0 mL to about 2 L, about 1.0 mL to about 1800 mL, about 1.0 mL to about 1500 mL, about 1.0 mL to about 1200 mL, about 1.0 mL to about 1000 mL, about 1.0 mL to about 800 mL, about 1.0 mL to about 600 mL, about 1.0 mL to about 500 mL, about 1.0 mL to about 450 mL, about 1.0 mL to about 400 mL, about 1.0 mL to about 350 mL, about 1.0 mL to about 300 mL, about 1.0 mL to about 250 mL, about 1.0 mL to about 200 mL, about 1.0 mL to about 180 mL, about 1.0 mL to about 160 mL, about 1.0 mL to about 140 mL, about 1.0 mL to about 120 mL, about 1.0 mL to about 100 mL, about 1.0 mL to about 80 mL, about 1.0 mL to about 60 mL, about 1.0 mL to about 40 mL, about 1.0 mL to about 20 mL, about 1.0 mL to about 10 mL, about 1.0 mL to about 5 mL, about 5 mL to about 2 L, about 5 mL to about 1800 mL, about 5 mL to about 1500 mL, about 5 mL to about 1200 mL, about 5 mL to about 1000 mL, about 5 mL to about 800 mL, about 5 mL to about 600 mL, about 5 mL to about 1800 mL, about 5 mL to about 500 mL, about 5 mL to about 450 mL, about 5 mL to about 400 mL, about 5 mL to about 350 mL, about 5 mL to about 300 mL, about 5 mL to about 250 mL, about 5 mL to about 200 mL, about 5 mL to about 180 mL, about 5 mL to about 160 mL, about 5 mL to about 140 mL, about 5 mL to about 120 mL, about 5 mL to about 100 mL, about 5 mL to about 80 mL, about 5 mL to about 60 mL, about 5 mL to about 40 mL, about 5 mL to about 20 mL, about 5 mL to about 10 mL, about 10 mL to about 2 L, about 10 mL to about 1800 mL, about 10 mL to about 1500 mL, about 10 mL to about 1200 mL, about 10 mL to about 1000 mL, about 10 mL to about 800 mL, about 10 mL to about 600 mL, about 10 mL to about 500 mL, about 10 mL to about 450 mL, about 10 mL to about 400 mL, about 10 mL to about 350 mL, about 10 mL to about 300 mL, about 10 mL to about 250 mL, about 10 mL to about 200 mL, about 10 mL to about 180 mL, about 10 mL to about 160 mL, about 10 mL to about 140 mL, about 10 mL to about 120 mL, about 10 mL to about 100 mL, about 10 mL to about 80 mL, about 10 mL to about 60 mL, about 10 mL to about 40 mL, about 10 mL to about 20 mL, about 20 mL to about 2 L, about 20 mL to about 1800 mL, about 20 mL to about 1500 mL, about 20 mL to about 1200 mL, about 20 mL to about 1000 mL, about 20 mL to about 800 mL, about 20 mL to about 600 mL, about 20 mL to about 500 mL, about 20 mL to about 450 mL, about 20 mL to about 400 mL, about 20 mL to about 350 mL, about 20 mL to about 300 mL, about 20 mL to about 250 mL, about 20 mL to about 200 mL, about 20 mL to about 180 mL, about 20 mL to about 160 mL, about 20 mL to about 140 mL, about 20 mL to about 120 mL, about 20 mL to about 100 mL, about 20 mL to about 80 mL, about 20 mL to about 60 mL, about 20 mL to about 40 mL, about 40 mL to about 2 L, about 40 mL to about 1800 mL, about 40 mL to about 1500 mL, about 40 mL to about 1200 mL, about 40 mL to about 1000 mL, about 40 mL to about 800 mL, about 40 mL to about 600 mL, about 40 mL to about 500 mL, about 40 mL to about 450 mL, about 40 mL to about 400 mL, about 40 mL to about 350 mL, about 40 mL to about 300 mL, about 40 mL to about 250 mL, about 40 mL to about 200 mL, about 40 mL to about 180 mL, about 40 mL to about 160 mL, about 40 mL to about 140 mL, about 40 mL to about 120 mL, about 40 mL to about 100 mL, about 40 mL to about 80 mL, about 40 mL to about 60 mL, about 60 mL to about 2 L, about 60 mL to about 1800 mL, about 60 mL to about 1500 mL, about 60 mL to about 1200 mL, about 60 mL to about 1000 mL, about 60 mL to about 800 mL, about 60 mL to about 600 mL, about 60 mL to about 500 mL, about 60 mL to about 450 mL, about 60 mL to about 400 mL, about 60 mL to about 350 mL, about 60 mL to about 300 mL, about 60 mL to about 250 mL, about 60 mL to about 200 mL, about 60 mL to about 180 mL, about 60 mL to about 160 mL, about 60 mL to about 140 mL, about 60 mL to about 120 mL, about 60 mL to about 100 mL, about 60 mL to about 80 mL, about 80 mL to about 2 L, about 80 mL to about 1800 mL, about 80 mL to about 1500 mL, about 80 mL to about 1200 mL, about 80 mL to about 1000 mL, about 80 mL to about 800 mL, about 80 mL to about 600 mL, about 80 mL to about 500 mL, about 80 mL to about 450 mL, about 80 mL to about 400 mL, about 80 mL to about 350 mL, about 80 mL to about 300 mL, about 80 mL to about 250 mL, about 80 mL to about 200 mL, about 80 mL to about 180 mL, about 80 mL to about 160 mL, about 80 mL to about 140 mL, about 80 mL to about 120 mL, about 80 mL to about 100 mL, about 100 mL to about 2 L, about 100 mL to about 1800 mL, about 100 mL to about 1500 mL, about 100 mL to about 1200 mL, about 100 mL to about 1000 mL, about 100 mL to about 800 mL, about 100 mL to about 600 mL, about 100 mL to about 500 mL, about 100 mL to about 450 mL, about 100 mL to about 400 mL, about 100 mL to about 350 mL, about 100 mL to about 300 mL, about 100 mL to about 250 mL, about 100 mL to about 200 mL, about 100 mL to about 180 mL, about 100 mL to about 160 mL, about 100 mL to about 140 mL, about 100 mL to about 120 mL, about 120 mL to about 2 L, about 120 mL to about 1800 mL, about 120 mL to about 1500 mL, about 120 mL to about 1200 mL, about 120 mL to about 1000 mL, about 120 mL to about 800 mL, about 120 mL to about 600 mL, about 120 mL to about 500 mL, about 120 mL to about 450 mL, about 120 mL to about 400 mL, about 120 mL to about 350 mL, about 120 mL to about 300 mL, about 120 mL to about 250 mL, about 120 mL to about 200 mL, about 120 mL to about 180 mL, about 120 mL to about 160 mL, about 120 mL to about 140 mL, about 140 mL to about 2 L, about 140 mL to about 1800 mL, about 140 mL to about 1500 mL, about 140 mL to about 1200 mL, about 140 mL to about 1000 mL, about 140 mL to about 800 mL, about 140 mL to about 600 mL, about 140 mL to about 500 mL, about 140 mL to about 450 mL, about 140 mL to about 400 mL, about 140 mL to about 350 mL, about 140 mL to about 300 mL, about 140 mL to about 250 mL, about 140 mL to about 200 mL, about 140 mL to about 180 mL, about 140 mL to about 160 mL, about 160 mL to about 500 mL, about 160 mL to about 450 mL, about 160 mL to about 400 mL, about 160 mL to about 350 mL, about 160 mL to about 300 mL, about 160 mL to about 250 mL, about 160 mL to about 200 mL, about 160 mL to about 180 mL, about 180 mL to about 2 L, about 180 mL to about 1800 mL, about 180 mL to about 1500 mL, about 180 mL to about 1200 mL, about 180 mL to about 1000 mL, about 180 mL to about 800 mL, about 180 mL to about 600 mL, about 180 mL to about 500 mL, about 180 mL to about 450 mL, about 180 mL to about 400 mL, about 180 mL to about 350 mL, about 180 mL to about 300 mL, about 180 mL to about 250 mL, about 180 mL to about 200 mL, about 200 mL to about 2 L, about 200 mL to about 1800 mL, about 200 mL to about 1500 mL, about 200 mL to about 1200 mL, about 200 mL to about 1000 mL, about 200 mL to about 800 mL, about 200 mL to about 600 mL, about 200 mL to about 500 mL, about 200 mL to about 450 mL, about 200 mL to about 400 mL, about 200 mL to about 350 mL, about 200 mL to about 300 mL, about 200 mL to about 250 mL, about 250 mL to about 2 L, about 250 mL to about 1800 mL, about 250 mL to about 1500 mL, about 250 mL to about 1200 mL, about 250 mL to about 1000 mL, about 250 mL to about 800 mL, about 250 mL to about 600 mL, about 250 mL to about 500 mL, about 250 mL to about 450 mL, about 250 mL to about 400 mL, about 250 mL to about 350 mL, about 250 mL to about 300 mL, about 300 mL to about 2 L, about 300 mL to about 1800 mL, about 300 mL to about 1500 mL, about 300 mL to about 1200 mL, about 300 mL to about 1000 mL, about 300 mL to about 800 mL, about 300 mL to about 600 mL, about 300 mL to about 500 mL, about 300 mL to about 450 mL, about 300 mL to about 400 mL, about 300 mL to about 350 mL, about 350 mL to about 2 L, about 350 mL to about 1800 mL, about 350 mL to about 1500 mL, about 350 mL to about 1200 mL, about 350 mL to about 1000 mL, about 350 mL to about 800 mL, about 350 mL to about 600 mL, about 350 mL to about 500 mL, about 350 mL to about 450 mL, about 350 mL to about 400 mL, about 400 mL to about 2 L, about 400 mL to about 1800 mL, about 400 mL to about 1500 mL, about 400 mL to about 1200 mL, about 400 mL to about 1000 mL, about 400 mL to about 800 mL, about 400 mL to about 600 mL, about 400 mL to about 500 mL, about 400 mL to about 450 mL, about 450 mL to about 2 L, about 450 mL to about 1800 mL, about 450 mL to about 1500 mL, about 450 mL to about 1200 mL, about 450 mL to about 1000 mL, about 450 mL to about 800 mL, about 450 mL to about 600 mL, about 450 mL to about 500 mL, about 500 mL to about 2 L, about 500 mL to about 1800 mL, about 500 mL to about 1500 mL, about 500 mL to about 1200 mL, about 500 mL to about 1000 mL, about 500 mL to about 800 mL, about 500 mL to about 600 mL, about 600 mL to about 2 L, about 600 mL to about 1800 mL, about 600 mL to about 1500 mL, about 600 mL to about 1200 mL, about 600 mL to about 1000 mL, about 600 mL to about 800 mL, about 800 mL to about 2 L, about 800 mL to about 1800 mL, about 800 mL to about 1500 mL, about 800 mL to about 1200 mL, about 800 mL to about 1000 mL, about 1000 mL to about 2 L, about 1000 mL to about 1800 mL, about 1000 mL to about 1500 mL, about 1000 mL to about 1200 mL, about 1200 mL to about 2 L, about 1200 mL to about 1800 mL, about 1200 mL to about 1500 mL, about 1500 mL to about 2 L, about 1500 mL to about 1800 mL, or about 1800 mL to about 2 L.

### Methods of Making a Formulation

Also provided herein are methods of making a composition that include: (i) providing a population of enucleated erythroid cells; and (ii) resuspending the population of enucleated erythroid cells in a pharmaceutically acceptable aqueous buffered solution (e.g., any of the exemplary pharmaceutically acceptable aqueous buffered solutions described herein). In some embodiments, the enucleated erythroid cells can be any of the enucleated erythroid cells described herein. In some embodiments, the composition can contain about 0.5×10⁸ to about 7.0×10⁹ enucleated erythroid cells/mL, e.g., about 0.5×10⁸ to about 6.0×10⁹, about 0.5×10⁸ to about 5.0×10⁹, about 0.5×10⁸ to about 4.0×10⁹, about 0.5×10⁸ to about 3.0×10⁹, about 0.5×10⁸ to about 2.0×10⁹, about 0.5×10⁸ to about 1.0×10⁹, about 0.5×10⁸ to about 0.5×10⁹, about 0.5×10⁸ to about 1.0×10⁸, about 1.0×10⁸ to about 7.0×10⁹, about 1.0×10⁸ to about 6.0×10⁹, about 1.0×10⁸ to about 5.0×10⁹, about 1.0×10⁸ to about 4.0×10⁹, about 1.0×10⁸ to about 3.0×10⁹, about 1.0×10⁸ to about 2.0×10⁹, about 1.0×10⁸ to about 1.0×10⁹, about 1.0×10⁸ to about 0.5×10⁹, about 0.5×10⁹ to about 7.0×10⁹, about 0.5×10⁹ to about 6.0×10⁹, about 0.5×10⁹ to about 5.0×10⁹, about 0.5×10⁹ to about 4.0×10⁹, about 0.5×10⁹ to about 3.0×10⁹, about 0.5×10⁹ to about 2.0×10⁹, about 0.5×10⁹ to about 1.0×10⁹, about 1.0×10⁹ to about 7.0×10⁹, about 1.0×10⁹ to about 6.0×10⁹, about 1.0×10⁹ to about 5.0×10⁹, about 1.0×10⁹ to about 4.0×10⁹, about 1.0×10⁹ to about 3.0×10⁹, about 1.0×10⁹ to about 2.0×10⁹, about 2.0×10⁹ to about 7.0×10⁹, about 2.0×10⁹ to about 6.0×10⁹, about 2.0×10⁹ to about 5.0×10⁹, about 2.0×10⁹ to about 4.0×10⁹, about 2.0×10⁹ to about 3.0×10⁹, about 3.0×10⁹ to about 7.0×10⁹, about 3.0×10⁹ to about 6.0×10⁹, about 3.0×10⁹ to about 5.0×10⁹, about 3.0×10⁹ to about 4.0×10⁹, about 4.0×10⁹ to about 7.0×10⁹, about 4.0×10⁹ to about 6.0×10⁹, about 4.0×10⁹ to about 5.0×10⁹, about 5.0×10⁹ to about 7.0×10⁹, about 5.0×10⁹ to about 6.0×10⁹, or about 6.0×10⁹ to about 7.0×10⁹ enucleated erythroid cells/mL (e.g., any of the exemplary enucleated erythroid cells described herein).

In some embodiments, the methods can include centrifuging the population of enucleated erythroid cells in step (i) before they are resuspended. Resuspending the enucleated erythroid cells in the pharmaceutically acceptable aqueous buffered solution in step (ii) may be performed by diafiltration or a combination of ultrafiltration/diafiltration. In some embodiments, the population of enucleated erythroid cells may be obtained from a donor (also donor enucleated erythroid cells, e.g., packed red blood cells).

In some embodiments, the population of enucleated erythroid cells may be washed red blood cells (RBCs). The washed red blood cells may be obtained from a donor and subsequently washed. Washing of RBCs obtained from a donor is typically performed using normal saline (0.9%) in either an open- or a closed-system. For example, the washing procedure removes ˜95%-99% of the RBC supernatant, which contains in addition to the additive solution, plasma proteins, electrolytes, some white blood cells (WBCs), platelets, microparticles, and cellular debris. RBCs washed in an open system are often used within 24 hours post-washing due to the theoretical increased risk for bacterial contamination, as well as RBC viability in normal saline. RBCs washed in a closed system have an expiration time of 14 days. RBC washing is frequently used in neonates and infants undergoing cardiac surgery. Washing of RBCs reduces extracellular potassium, but increases the RBC membrane osmotic fragility, which leads to increased hemolysis of the washed RBCs within the first three days of neonatal extracorporeal membrane oxygenation (ECMO). RBC washing may also increase the RBC osmotic fragility, leading to increased hemolysis following transfusion. Non-limiting examples of RBC washing devices include the Cobe 2991 (Terumo BCT, Lakewood, Colo., USA) and the Haemonetics Cell Saver Elite (Haemonetics, Braintree, Mass., USA). Additional non-limiting aspects of the washing of RBCs are described in Schmidt et al. (Int. J. Clin. Transfusion Med. 4:79-88, 2016).

In some embodiments, the enucleated erythroid cells are engineered enucleated erythroid cells (e.g., any of the exemplary engineered enucleated erythroid cells described herein).

Some embodiments of any of these methods further include culturing erythroid progenitor cells to provide the population of enucleated erythroid cells.

In some embodiments, where the engineered erythroid cells are engineered enucleated erythroid cells, the methods can further include introducing into an erythroid progenitor cell one or more nucleic acids (e.g., any of the exemplary nucleic acids described herein) encoding one or more exogenous proteins (e.g., any of the exemplary exogenous proteins described herein) (e.g., using any of the methods of introducing a nucleic acid into an erythroid progenitor cell described herein). In these examples, the method can further include culturing the erythroid progenitor cell before and/or after the introduction of the nucleic acid into the erythroid progenitor cell.

Erythroid progenitor cells can be patient-derived erythroid progenitor cells, immortalized erythroid cell lines, or can be derived from induced pluripotent stem cells. For example, in some embodiments, the erythroid progenitor cells are immortalized erythroid cell lines, e.g., cell lines comprising at least one exogenous nucleic acid encoding human papilloma virus (HPV) E6 and/or HPV E7. In some embodiments, the erythroid progenitor cell comprises at least one exogenous nucleic acid encoding one or more of Oct4, Sox2, Klf4, and cMyc, and optionally comprises a genetic modification to suppress, reduce or ablate the expression of TP53 (see e.g. Huang et al., (2014) Mol. Ther. 22(2): 451-63). In some embodiments, the erythroid progenitor cell is a BEL-A cell line cell (see Trakarnasanga et al. (2017) Nat. Commun. 8: 14750). Additional immortalized erythroid progenitor cells are described in U.S. Pat. Nos. 9,951,350, and 8,975,072. In some embodiments, the erythroid progenitor cell comprises at least one exogenous nucleic acid encoding Bmi-1. Exemplary methods for generating enucleated erythroid cells using cell culture techniques are well known in the art, e.g., Giarratana et al., Blood 118:5071, 2011; Kurita et al., PLOS One 8:e59890, 2013; Fibach et al., Blood 73:100, 1989; Giarratana et al., Blood 118:5071, 2011). Enucleated erythroid cells can be produced by culturing hematopoietic progenitor cells, including, for example, CD34⁺ hematopoietic progenitor cells (Giarratana et al., Blood 118:5071, 2011), induced pluripotent stem cells (Kurita et al., PLOS 8:e59890, 2013), and embryonic stem cells (Hirose et al., Stem Cell Reports 1:499, 2013). Cocktails of growth and differentiation factors that are suitable to expand and differentiate erythroid progenitor cells into enucleated erythroid cells are known in the art. Examples of suitable expansion and differentiation factors include, but are not limited to, stem cell factor (SCF), an interleukin (IL), such as IL-1, IL-2, IL-3, IL-4, IL-5, IL-6, IL-7, IL-8, IL-9, IL-11, IL-12, CSF, G-CSF, thrombopoietin (TPO), GM-CSF, erythropoietin (EPO), Flt3, Flt2, PIXY 321, and leukemia inhibitory factor (LIF).

Erythroid cells can be cultured from erythroid progenitor cells (e.g., any of the exemplary erythroid progenitor cells described herein), such as CD34⁺ cells, by contacting the erythroid progenitor cells with defined factors in a multi-step culture process. For example, enucleated erythroid cells can be generated from erythroid progenitor cells in a three-step culture process.

The first step can include culturing erythroid progenitor cells in a liquid culture medium including stem cell factor (SCF) at 1-1000 ng/mL, erythropoietin (EPO) at 1-100 U/mL, and interleukin-3 (IL-3) at 0.1-100 ng/mL. In the first step, the liquid culture medium can further include a ligand that binds and activates a nuclear hormone receptor, such as e.g., the glucocorticoid receptor, the estrogen receptor, the progesterone receptor, the androgen receptor, or the pregnane X receptor. The ligands for these receptors include, for example, a corticosteroid, such as, e.g., dexamethasone at 10 nM-100 μM or hydrocortisone at 10 nM-100 μM; an estrogen, such as, e.g., beta-estradiol at 10 nM-100 μM; a progestogen, such as, e.g., progesterone at 10 nM-100 μM, hydroxyprogesterone at 10 nM-100 μM, 5a-dihydroprogesterone at 10 nM-100 μM, 11-deoxycorticosterone at 10 nM-100 μM, or a synthetic progestin, such as, e.g., chlormadinone acetate at 10 nM-100 μM; an androgen, such as, e.g., testosterone at 10 nM-100 μM, dihydrotestosterone at 10 nM-100 μM or androstenedione at 10 nM-100 μM; or a pregnane X receptor ligand, such as, e.g., rifampicin at 10 nM-100 μM, hyperforin at 10 nM-100 μM, St. John's Wort (hypericin) at 10 nM-100 μM, or vitamin E-like molecules, such as, e.g., tocopherol at 10 nM-100 μM. The liquid culture medium in the first step can also include an insulin-like molecule, such as, e.g., insulin at 1-50 μg/mL, insulin-like growth factor 1 (IGF-1) at 1-50 μg/mL, insulin-like growth factor 2 (IGF-2) at 1-50 μg/mL, or mechano-growth factor at 1-50 μg/mL. The liquid culture medium in the first step can also include transferrin at 0.1-5 mg/mL.

The liquid culture medium used in the first step can optionally include one or more interleukins (IL) or growth factors such as, e.g., IL-1, IL-2, IL-4, IL-5, IL-6, IL-7, IL-8, IL-9, IL-11, IL-12, granulocyte colony-stimulating factor (G-CSF), macrophage colonystimulating factor (M-CSF), granulocyte-macrophage colony-stimulating factor (GM-CSF), thrombopoietin, fibroblast growth factor (FGF), platelet-derived growth factor (PDGF), transforming growth factor beta (TGF-B), tumor necrosis factor alpha (TNF-A), megakaryocyte growth and development factor (MGDF), leukemia inhibitory factor (LIF), and Flt3 ligand. Each interleukin or growth factor may be included in the liquid culture medium used in the first step at a concentration of 0.1-100 ng/mL. The liquid culture medium used in the first step may also optionally include serum proteins or non-protein molecules such as, e.g., human serum (1-20%), human plasma (1-20%), plasmanate (1-20%), human serum (1-20%), human albumin (0.1-100 mg/mL), or heparin (0.1-10 U/mL).

The second step of the three-step culturing process includes culturing the progenitor cells in a liquid culture medium including stem cell factor (SCF) at 1-1000 ng/mL and erythropoietin (EPO) at 1-100 U/mL. The liquid culture medium used in the second step can also optionally include an insulin-like molecule, such as e.g., insulin at 1-50 μg/mL, insulin-like growth factor 1 (IGF-1) at 1-50 μg/mL, insulin-like growth factor 2 (IGF-2) at 1-50 μg/mL, or mechano-growth factor at 1-50 μg/mL. The liquid culture medium used in the second step can optionally further include transferrin at 0.1-5 mg/mL. The liquid culture medium used in the second step can optionally further include serum proteins or non-protein molecules such as, e.g., human plasma (1-20%), plasmanate (1-20%), human serum (1-20%), human albumin (0.1-100 mg/mL), or heparin (0.1-10 U/mL).

The third step of the three-step culture process includes culturing the erythroid progenitor cells in a liquid culture medium including erythropoietin (EPO) at 1-100 U/mL. The liquid culture medium used in the third step can optionally further stem cell factor (SCF) at 1-1000 ng/mL. The liquid culture medium used in the third step can optionally further include an insulin-like molecule, such as e.g., insulin at 1-50 μg/mL, insulin-like growth factor 1 (IGF-1) at 1-50 μg/mL, insulin-like growth factor 2 (IGF-2) at 1-50 μg/mL, or mechano-growth factor at 1-50 μg/mL. The liquid culture medium used in the third step can also optionally include transferrin at 0.1-5 mg/mL and/or serum proteins or non-protein molecules such as, e.g., human serum (1-20%), human plasma (1-20%), plasmanate (1-20%), human serum (1-20%), human albumin (0.1-100 mg/mL), or heparin (0.1-10 U/mL).

In some embodiments, the methods further include disposing the composition into a sterile vessel. Non-limiting examples of sterile vessels include a sterile conical tube, a sterile petri dish, a sterile vial (e.g., a borosilicate glass vial), and sterile plastic bags (a di-2-ethylhexyl phthalate (DEHP)-plasticized polyvinyl chloride (PVC) bag, or n-butyryl-tri(n-hexyl)-citrate (BTHC)-plasticized PVC bag).

### Methods of Treating a Subject

Also provided herein are methods of treating a subject in need thereof (e.g., any of the subjects described herein or known in the art) that include: (i) providing a composition (e.g. any of the compositions described herein) that has been stored at a temperature of about 2° C. to about 10° C. (e.g., about 2° C. to about 9° C., about 2° C. to about 8° C., about 2° C. to about 7° C., about 2° C. to about 6° C., about 2° C. to about 5° C., about 2° C. to about 4° C., about 3° C. to about 10° C., about 3° C. to about 9° C., about 3° C. to about 8° C., about 3° C. to about 7° C., about 3° C. to about 6° C., about 3° C. to about 5° C., about 4° C. to about 10° C., about 4° C. to about 9° C., about 4° C. to about 8° C., about 4° C. to about 7° C., about 4° to about 6° C., about 5° C. to about 10° C., about 5° C. to about 9° C., about 5° C. to about 8° C., about 5° C. to about 7° C., about 6° C. to about 10° C., about 6° C. to about 9° C., about 6° C. to about 8° C., about 7° C. to about 10° C., about 7° C. to about 9° C., or about 8° C. to about 10° C.) for a period of time; (ii) administering the composition of step (ii) to a subject in need thereof. In some embodiments of these methods, the subject has been previously identified or diagnosed as being in need of one or more of the exogenous proteins present in the administered engineered enucleated erythroid cells. In some embodiments, the subject has been previously identified or diagnosed as being in need of a blood transfusion and/or an increase in erythrocytes.

In some embodiments, provided herein are methods of treating a subject having phenylketonuria that include: (i) providing a composition where the engineered human enucleated erythroid cells comprise exogenous protein phenylalanine ammonia lyase (PAL), that has been stored at a temperature of about 2° C. to about 10° C. (e.g., about 2° C. to about 9° C., about 2° C. to about 8° C., about 2° C. to about 7° C., about 2° C. to about 6° C., about 2° C. to about 5° C., about 2° C. to about 4° C., about 3° C. to about 10° C., about 3° C. to about 9° C., about 3° C. to about 8° C., about 3° C. to about 7° C., about 3° C. to about 6° C., about 3° C. to about 5° C., about 4° C. to about 10° C., about 4° C. to about 9° C., about 4° C. to about 8° C., about 4° C. to about 7° C., about 4° to about 6° C., about 5° C. to about 10° C., about 5° C. to about 9° C., about 5° C. to about 8° C., about 5° C. to about 7° C., about 6° C. to about 10° C., about 6° C. to about 9° C., about 6° C. to about 8° C., about 7° C. to about 10° C., about 7° C. to about 9° C., or about 8° C. to about 10° C.) for a period of time; and (ii) administering the composition of step (i) to the subject. In some embodiments of these methods, the subject has been previously identified or diagnosed as having disease phenylketonuria.

In some embodiments, provided herein are methods of treating a subject having a cancer that include: (i) providing a composition where the engineered human enucleated erythroid cells comprise a combination of a first exogenous protein comprising an antigenic peptide (e.g., an HPV antigen such as HPV16 E7₁₁₋₁₉) linked to beta 2 microglobulin (B2M), or a fragment thereof, linked to one or more of the alpha1, alpha2, and alpha 3 domains of an MHC class I protein (e.g., HLA*02:01), or fragment or variant thereof, linked to a transmembrane protein (e.g., GPA, or a transmembrane fragment thereof); a second exogenous protein comprising 4-1BBL, or a fragment thereof, linked to a transmembrane protein (e.g., GPA, or a transmembrane fragment thereof); and a second exogenous protein comprising IL-12 p40, or a fragment thereof, linked to IL-12 p35, or a fragment thereof, linked to a transmembrane protein (e.g., GPA, or a transmembrane fragment thereof, or SMIM1, or a transmembrane fragment thereof) (e.g., as described in U.S. Patent Application Publication No. 2019/0290686, incorporated herein by reference), that has been stored at a temperature of about 2° C. to about 10° C. (e.g., about 2° C. to about 9° C., about 2° C. to about 8° C., about 2° C. to about 7° C., about 2° C. to about 6° C., about 2° C. to about 5° C., about 2° C. to about 4° C., about 3° C. to about 10° C., about 3° C. to about 9° C., about 3° C. to about 8° C., about 3° C. to about 7° C., about 3° C. to about 6° C., about 3° C. to about 5° C., about 4° C. to about 10° C., about 4° C. to about 9° C., about 4° C. to about 8° C., about 4° C. to about 7° C., about 4° to about 6° C., about 5° C. to about 10° C., about 5° C. to about 9° C., about 5° C. to about 8° C., about 5° C. to about 7° C., about 6° C. to about 10° C., about 6° C. to about 9° C., about 6° C. to about 8° C., about 7° C. to about 10° C., about 7° C. to about 9° C., or about 8° C. to about 10° C.) for a period of time; and (ii) administering the composition of step (i) to the subject. In some embodiments of these methods, the subject has been previously identified or diagnosed as having cancer.

In some embodiments, provided herein are methods of treating a subject having a cancer that include: (i) providing a composition where the engineered human enucleated erythroid cells comprise a combination of: a first exogenous protein comprising 4-1BBL, or a fragment thereof, linked to a transmembrane protein (e.g., GPA, or a transmembrane fragment thereof); and a second exogenous protein comprising IL-15, or a fragment thereof, linked to an extracellular portion of IL-15 receptor alpha (IL-15Rα), or a fragment thereof (e.g., an IL-15Rα sushi-binding domain), linked to a transmembrane protein (e.g., GPA, or a transmembrane fragment thereof) (e.g., as described in U.S. Patent Application Publication No. 2019/0298769, incorporated herein by reference), that has been stored at a temperature of about 2° C. to about 10° C. (e.g., about 2° C. to about 9° C., about 2° C. to about 8° C., about 2° C. to about 7° C., about 2° C. to about 6° C., about 2° C. to about 5° C., about 2° C. to about 4° C., about 3° C. to about 10° C., about 3° C. to about 9° C., about 3° C. to about 8° C., about 3° C. to about 7° C., about 3° C. to about 6° C., about 3° C. to about 5° C., about 4° C. to about 10° C., about 4° C. to about 9° C., about 4° C. to about 8° C., about 4° C. to about 7° C., about 4° to about 6° C., about 5° C. to about 10° C., about 5° C. to about 9° C., about 5° C. to about 8° C., about 5° C. to about 7° C., about 6° C. to about 10° C., about 6° C. to about 9° C., about 6° C. to about 8° C., about 7° C. to about 10° C., about 7° C. to about 9° C., or about 8° C. to about 10° C.) for a period of time; and (ii) administering the composition of step (i) to the subject. In some embodiments of these methods, the subject has been previously identified or diagnosed as having cancer.

In some embodiments, provided herein are methods of treating a subject having any of the diseases or conditions listed in Tables A through D that include: (i) providing a composition where the engineered human enucleated erythroid cells comprises an exogenous protein (e.g., one or more of the exemplary exogenous proteins listed in Tables A through D for treatment of the corresponding disease or condition listed in Tables A through D), that has been stored at a temperature of about 2° C. to about 10° C. (e.g., about 2° C. to about 9° C., about 2° C. to about 8° C., about 2° C. to about 7° C., about 2° C. to about 6° C., about 2° C. to about 5° C., about 2° C. to about 4° C., about 3° C. to about 10° C., about 3° C. to about 9° C., about 3° C. to about 8° C., about 3° C. to about 7° C., about 3° C. to about 6° C., about 3° C. to about 5° C., about 4° C. to about 10° C., about 4° C. to about 9° C., about 4° C. to about 8° C., about 4° C. to about 7° C., about 4° to about 6° C., about 5° C. to about 10° C., about 5° C. to about 9° C., about 5° C. to about 8° C., about 5° C. to about 7° C., about 6° C. to about 10° C., about 6° C. to about 9° C., about 6° C. to about 8° C., about 7° C. to about 10° C., about 7° C. to about 9° C., or about 8° C. to about 10° C.) for a period of time; and (ii) administering the composition of step (i) to the subject in need thereof. In some embodiments of these methods, the subject has been previously identified or diagnosed as having disease or condition listed in Tables A through D.

Some embodiments of these methods further include between step (i) and step (ii) a step of warming the composition of step (i) to a temperature of about 15° C. to about 30° C. (e.g. about 15° C. to about 29° C., about 15° C. to about 28° C., about 15° C. to about 27° C., about 15° C. to about 26° C., about 15° C. to about 25° C., about 15° C. to about 24° C., about 15° C. to about 23° C., about 15° C. to about 22° C., about 15° C. to about 21° C., about 15° C. to about 20° C., about 15° C. to about 19° C., about 15° C. to about 18° C., about 15° C. to about 17° C., about 15° C. to about 16° C., about 16° C. to about 30° C., about 16° C. to about 29° C., about 16° C. to about 28° C., about 16° C. to about 27° C., about 16° C. to about 26° C., about 16° C. to about 25° C., about 16° C. to about 24° C., about 16° C. to about 23° C., about 16° C. to about 22° C., about 16° C. to about 21° C., about 16° C. to about 20° C., about 16° C. to about 19° C., about 16° C. to about 18° C., about 16° C. to about 17° C., about 17° C. to about 30° C., about 17° C. to about 29° C., about 17° C. to about 28° C., about 17° C. to about 27° C., about 17° C. to about 26° C., about 17° C. to about 25° C., about 17° C. to about 24° C., about 17° C. to about 23° C., about 17° C. to about 22° C., about 17° C. to about 21° C., about 17° C. to about 20° C., about 17° C. to about 19° C., about 17° C. to about 18° C., about 18° C. to about 30° C., about 18° C. to about 29° C., about 18° C. to about 28° C., about 18° C. to about 27° C., about 18° C. to about 26° C., about 18° C. to about 25° C., about 18° C. to about 24° C., about 18° C. to about 23° C., about 18° C. to about 22° C., about 18° C. to about 21° C., about 18° C. to about 20° C., about 18° C. to about 19° C., about 19° C. to about 30° C., about 19° C. to about 29° C., about 19° C. to about 28° C., about 19° C. to about 27° C., about 19° C. to about 26° C., about 19° C. to about 25° C., about 19° C. to about 24° C., about 19° C. to about 23° C., about 19° C. to about 22° C., about 19° C. to about 21° C., about 19° C. to about 20° C., about 20° C. to about 30° C., about 20° C. to about 29° C., about 20° C. to about 28° C., about 20° C. to about 27° C., about 20° C. to about 26° C., about 20° C. to about 25° C., about 20° C. to about 24° C., about 20° C. to about 23° C., about 20° C. to about 22° C., about 20° C. to about 21° C., about 21° C. to about 30° C., about 21° C. to about 29° C., about 21° C. to about 28° C., about 21° C. to about 27° C., about 21° C. to about 26° C., about 21° C. to about 25° C., about 21° C. to about 24° C., about 21° C. to about 23° C., about 21° C. to about 22° C., about 22° C. to about 30° C., about 22° C. to about 29° C., about 22° C. to about 28° C., about 22° C. to about 27° C., about 22° C. to about 26° C., about 22° C. to about 25° C., about 22° C. to about 24° C., about 22° C. to about 23° C., about 23° C. to about 30° C., about 23° C. to about 29° C., about 23° C. to about 28° C., about 23° C. to about 27° C., about 23° C. to about 26° C., about 23° C. to about 25° C., about 23° C. to about 24° C., about 24° C. to about 30° C., about 24° C. to about 29° C., about 24° C. to about 28° C., about 24° C. to about 27° C., about 24° C. to about 26° C., about 24° C. to about 25° C., about 25° C. to about 30° C., about 25° C. to about 29° C., about 25° C. to about 28° C., about 25° C. to about 27° C., about 25° C. to about 26° C., about 26° C. to about 30° C., about 26° C. to about 29° C., about 26° C. to about 28° C., about 26° C. to about 27° C., about 27° C. to about 30° C., about 27° C. to about 29° C., about 27° C. to about 28° C., about 28° C. to about 30° C., about 28° C. to about 29° C., or about 29° C. to about 30° C.).

In some embodiments of these methods, the period of time is about 30 days to about 100 days (e.g., about 30 days to about 95 days, about 30 days to about 90 days, about 30 days to about 85 days, about 30 days to about 80 days, about 30 days to about 75 days, about 30 days to about 70 days, about 30 days to about 65 days, about 30 days to about 60 days, about 30 days to about 55 days, about 30 days to about 50 days, about 30 days to about 45 days, about 30 days to about 40 days, about 30 days to about 35 days, about 35 days to about 100 days, about 35 days to about 95 days, about 35 days to about 90 days, about 35 days to about 85 days, about 35 days to about 80 days, about 35 days to about 75 days, about 35 days to about 70 days, about 35 days about to 65 days, about 35 days to about 60 days, about 35 days to about 55 days, about 35 days to about 50 days, about 35 days to about 45 days, about 35 days to about 40 days, about 40 days to about 100 days, about 40 days to about 95 days, about 40 days to about 90 days, about 40 days to about 85 days, about 40 days to about 80 days, about 40 days to about 75 days, about 40 days to about 70 days, about 40 days about to 65 days, about 40 days to about 60 days, about 40 days to about 55 days, about 40 days to about 50 days, about 40 days to about 45 days, about 45 days to about 100 days, about 45 days to about 95 days, about 45 days to about 90 days, about 45 days to about 85 days, about 45 days to about 80 days, about 45 days to about 75 days, about 45 days to about 70 days, about 45 days about to 65 days, about 45 days to about 60 days, about 45 days to about 55 days, about 45 days to about 50 days, about 50 days to about 100 days, about 50 days to about 95 days, about 50 days to about 90 days, about 50 days to about 85 days, about 50 days to about 80 days, about 50 days to about 75 days, about 50 days to about 70 days, about 50 days about to 65 days, about 50 days to about 60 days, about 50 days to about 55 days, about 55 days to about 100 days, about 55 days to about 95 days, about 55 days to about 90 days, about 55 days to about 85 days, about 55 days to about 80 days, about 55 days to about 75 days, about 55 days to about 70 days, about 55 days about to 65 days, about 55 days to about 60 days, about 60 days to about 100 days, about 60 days to about 95 days, about 60 days to about 90 days, about 60 days to about 85 days, about 60 days to about 80 days, about 60 days to about 75 days, about 60 days to about 70 days, about 60 days about to 65 days, about 65 days to about 100 days, about 65 days to about 95 days, about 65 days to about 90 days, about 65 days to about 85 days, about 65 days to about 80 days, about 65 days to about 75 days, about 65 days to about 70 days, about 70 days to about 100 days, about 70 days to about 95 days, about 70 days to about 90 days, about 70 days to about 85 days, about 70 days to about 80 days, about 70 days to about 75 days, about 75 days to about 100 days, about 75 days to about 95 days, about 75 days to about 90 days, about 75 days to about 85 days, about 75 days to about 80 days, about 80 days to about 100 days, about 80 days to about 95 days, about 80 days to about 90 days, about 80 days to about 85 days, about 85 days to about 100 days, about 85 days to about 95 days, about 85 days to about 90 days, about 90 days to about 100 days, about 90 days to about 95 days, about 95 days to about 100 days).

In some embodiments of these methods, step (ii) includes intravenous administration to the subject.

Also provided herein are methods of treating a subject that include administering any of the compositions described herein to a subject in need thereof (e.g., a subject previously identified or diagnosed as being in need of the one or more exogenous proteins present in an engineered enucleated erythroid cell, or a subject identified as being in need of a blood transfusion and/or an increase in erythrocytes). In some embodiments of these methods, the composition was previously stored at a temperature of about 2° C. to about 10° C. (e.g., about 2° C. to about 9° C., about 2° C. to about 8° C., about 2° C. to about 7° C., about 2° C. to about 6° C., about 2° C. to about 5° C., about 2° C. to about 4° C., about 3° C. to about 10° C., about 3° C. to about 9° C., about 3° C. to about 8° C., about 3° C. to about 7° C., about 3° C. to about 6° C., about 3° C. to about 5° C., about 4° C. to about 10° C., about 4° C. to about 9° C., about 4° C. to about 8° C., about 4° C. to about 7° C., about 4° to about 6° C., about 5° C. to about 10° C., about 5° C. to about 9° C., about 5° C. to about 8° C., about 5° C. to about 7° C., about 6° C. to about 10° C., about 6° C. to about 9° C., about 6° C. to about 8° C., about 7° C. to about 10° C., about 7° C. to about 9° C., or about 8° C. to about 10° C.) for a period of time. In some embodiments, the period of time is about 30 days to about 100 days (e.g., any of the subranges of this range described herein).

Some embodiments of these methods further include prior to the administering step, a step of warming the composition to a temperature of about 15° C. to about 30° C. (e.g., any of the subranges of this range described herein). In some embodiments of these methods, the administering step comprises intravenous administration to the subject.

Some embodiments of any of the methods described herein further include administering one or more additional therapeutic agents to the subject. In some embodiments, the one or more additional therapeutic agents can be administered to the subject at the substantially the same time as any of the compositions provided herein. In some embodiments, the one or more additional therapeutic agents can be administered to the subject before or after the administration of any of the compositions described herein to the subject.

### EXAMPLES

The invention is further described in the following examples, which do not limit the scope of the invention described in the claims.

### Example 1: Formulation Development

Formulation development for enucleated erythroid cells was carried out using design of experiment (DOE) methods. The T1 and T2 formulation series (see Tables 1 and 2) were developed. The T1 formulation series was found to be superior to HypoThermosol® (HTS; Sigma-Aldrich Cat. No. H4416), which is a known formulation developed for hypothermic storage of cells, which includes 5 mM glucose and includes a colloid. A formulation designated as T1-1 was identified as being the most superior as determined by cell count, hemolysis, and cell deformability after storage. Surprisingly, T1-1 was a glucose-free formulation, which was unexpected since glucose is thought to be critical for stabilization of enucleated erythroid cells during storage.

TABLE 1
T1 Formulations

| Components | | | | | | | | | | T1- | T1- | T1- | |
|---|---|---|---|---|---|---|---|---|---|---|---|---|---|
| (mM) | T1-1 | T1-2 | T1-3 | T1-4 | T1-5 | T1-6 | T1-7 | T1-8 | T1-9 | 6a5 | 6a0 | 6m | T1-6c |
| Na⁺ | 80 | 100 | 106 | 100 | 106 | 80 | 106 | 80 | 100 | 80 | 80 | 80 | 80 |
| K⁺ | 42 | 42 | 42 | 42 | 42 | 42 | 42 | 42 | 42 | 42 | 42 | 42 | 42 |
| Ca²⁺ | 0.05 | 0.05 | 0.05 | 0.05 | 0.05 | 0.05 | 0.05 | 0.05 | 0.05 | 0.05 | 0.05 | 0.05 | 0.2 |
| Mg²⁺ | 5 | 5 | 5 | 5 | 5 | 5 | 5 | 5 | 5 | 5 | 5 | 10 | 5 |
| Cl⁻ | 10.1 | 10.1 | 10.1 | 10.1 | 10.1 | 10.1 | 10.1 | 10.1 | 10.1 | 10.1 | 10.1 | 10.1 | 10.4 |
| HPO₄²⁻ | 10 | 20 | 40 | 20 | 40 | 10 | 40 | 10 | 20 | 10 | 10 | 10 | 10 |
| HCO³⁻ | 5 | 5 | 5 | 5 | 5 | 5 | 5 | 5 | 5 | 5 | 5 | 5 | 5 |
| HEPES | 25 | 25 | 25 | 25 | 25 | 25 | 25 | 25 | 25 | 25 | 25 | 25 | 25 |
| Lactobionate | 100 | 100 | 70 | 100 | 70 | 100 | 70 | 100 | 100 | 100 | 100 | 100 | 100 |
| Mannitol | 40 | 20 | 0 | 0 | 40 | 20 | 20 | 0 | 40 | 20 | 20 | 20 | 20 |
| Adenosine | 2 | 2 | 2 | 2 | 2 | 2 | 2 | 2 | 2 | 5 | 0 | 2 | 2 |
| Adenine | 1 | 2 | 3 | 1 | 2 | 3 | 1 | 2 | 3 | 3 | 3 | 3 | 3 |
| Glucose | 0 | 0 | 0 | 5 | 5 | 5 | 10 | 10 | 10 | 5 | 5 | 5 | 5 |
| Osmolarity | 320 | 331 | 305 | 315 | 352 | 307 | 336 | 291 | 362 | 310 | 305 | 322 | 308 |
| (mOsm/L) | | | | | | | | | | | | | |
| pH | 7.57 | 7.63 | 7.51 | 7.62 | 7.50 | 7.47 | 7.53 | 7.53 | 7.55 | 7.51 | 7.53 | 7.49 | 7.51 |

TABLE 2
T2 Formulations

| Components | | | | | | | | | |
|---|---|---|---|---|---|---|---|---|---|
| (mM) | T2-1 | T2-2 | T2-3 | T2-4 | T2-5 | T2-6 | T2-7 | T2-8 | T2-9 |
| Na⁺ | 120 | 125 | 121 | 142 | 139 | 138 | 122 | 121 | 121 |
| K⁺ | 10 | 10 | 10 | 10 | 10 | 10 | 10 | 10 | 10 |
| Ca²⁺ | 0.05 | 0.05 | 0.05 | 0.05 | 0.05 | 0.05 | 0.05 | 0.05 | 0.05 |
| Mg²⁺ | 5 | 5 | 5 | 5 | 5 | 5 | 5 | 5 | 5 |
| Cl⁻ | 10.1 | 10.1 | 10.1 | 10.1 | 10.1 | 10.1 | 10.1 | 10.1 | 10.1 |
| H2PO⁴⁻ | 25 | 25 | 25 | 20 | 20 | 20 | 10 | 10 | 10 |
| HCO³⁻ | 5 | 5 | 5 | 5 | 5 | 5 | 5 | 5 | 5 |
| HEPES | 25 | 40 | 25 | 40 | 25 | 40 | 40 | 25 | 25 |
| Lactobionate | 100 | 100 | 70 | 100 | 70 | 100 | 70 | 100 | 100 |
| Mannitol | 40 | 20 | 40 | 20 | 40 | 40 | 40 | 40 | 20 |
| Adenosine | 2 | 2 | 2 | 2 | 2 | 2 | 2 | 2 | 2 |
| Adenine | 1 | 2 | 2.5 | 1 | 2 | 2.5 | 1 | 2 | 2.5 |
| Glucose | 0 | 0 | 0 | 0 | 0 | 0 | 0 | 0 | 0 |
| Osmolarity | 318 | 320 | 320 | 355 | 358 | 373 | 345 | 330 | 310 |
| (mOsm/L) | | | | | | | | | |
| pH | 7.39 | 7.36 | 7.43 | 7.36 | 7.55 | 7.37 | 7.39 | 7.47 | 7.45 |

### Example 2: Stability Analysis of Cell Storage Formulations

### Materials and Methods

### Hemolysis Assay

To calculate percent hemolysis (%), the following function was used:

Percentage hemolysis (%)=Supernatant hemoglobin/Total hemoglobin where hemoglobin was measured according to the Harboe direct spectrophotometric method (Han et al., Vox Sang 98(2):116-23, 2010).

Hemoglobin=1.539×[1.672×(A₄₁₅)−0.836×(A₃₈₀)−0.836×(A₄₅₀)], where A₄₁₅, A₃₈₀, and A₄₅₀ were light absorbance at 415 nm, 380 nm, and 450 nm respectively, measured using a BioTek Gen5 plate reader.

### Results

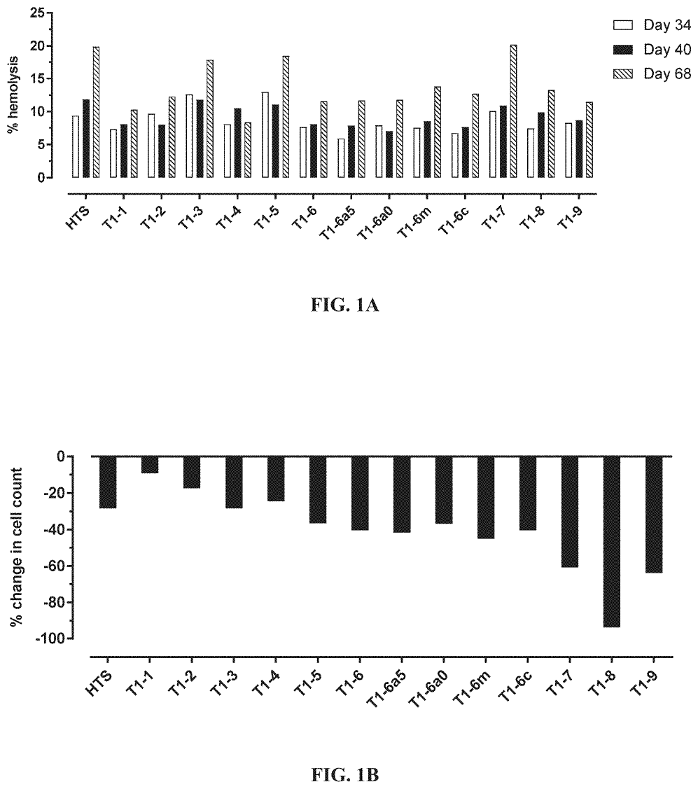

The percent hemolysis and change in cell count of an exemplary engineered enucleated erythroid cell population comprising phenylalanine ammonia lyase (PAL) stored in various formulations including HTS and formulations in the T1 series were analyzed over the course of 68 days. The formulations in the T1 series that were tested include T1-1, T1-2, T1-3, T1-4, T1-5, T1-6, T1-6a5, T1-6a0, T1-6m, T1-6c, T1-7, T1-8, and T1-9. The T1-7, T1-8, and T1-9 formulations contained higher levels of glucose as compared to the remaining formulations, including HTS. As shown in FIG. 1A, various T1 series formulations exhibited reduced enucleated erythroid cell hemolysis as compared to HTS after storage for 34, 40, and 68 days. In particular, storage in T1-1 for 68 days resulted in lower hemolylsis as compared to HTS. Next, cell count was analyzed for enucleated erythroid cells that have been stored for 68 days. As shown in FIG. 1B, storage in T1-1 also resulted in a lower decrease in cell count as compared to storage in the HTS solutions. Surprisingly, storage in T1-7, T1-8, and T1-9 which contain higher levels of glucose as compared to the other formulations resulted in the highest decrease in cell count.

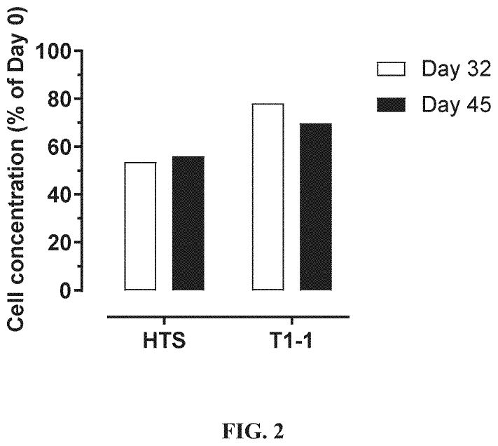

The enucleated erythroid cell concentration after 32 and 45 days of storage in HTS or T1-1 were also analyzed. The cell concentration was measured as the ratio of cell count following storage of 32 or 45 days to the cell count prior to storage. As shown in FIG. 2, T1-1 resulted in less of a decrease in cell concentration as compared to HTS at both 32 days and 45 days.

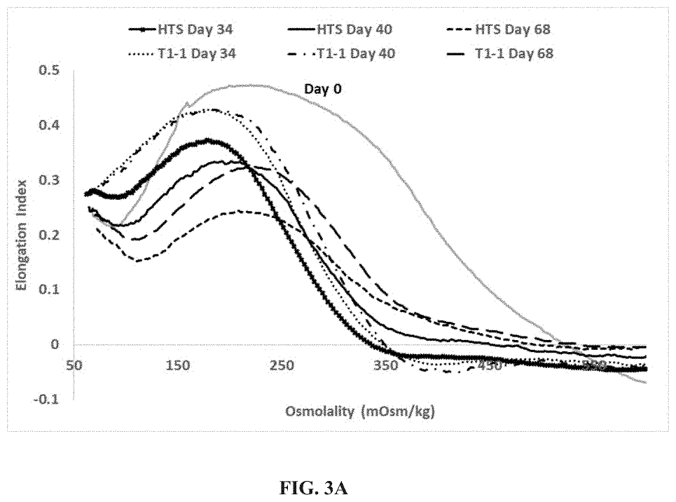

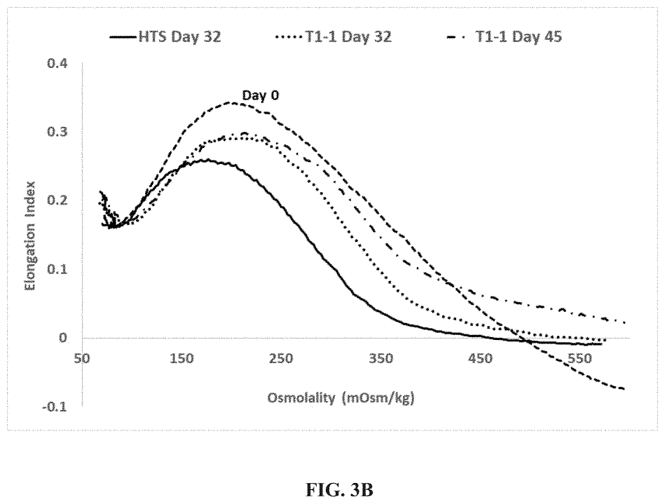

Next, the osmocan properties of enucleated erythroid cells stored in HTS or T1-1 for 34, 40, and 68 days were analyzed by ektacytometry using a laser-assisted rotational red cell analyser (Lorrca® Maxsis). As shown in FIG. 3A, enucleated erythroid cells stored in T1-1 maintained significantly higher EImax (peak elongation index) and area-under-curve (AUC) as compared to enucleated erythroid cells stored in HTS at all three time points studied. Specifically, cells stored for 68 days in T1-1 showed osmoscan properties similar to cells stored in HTS for 40 days.

The osmocan properties of enucleated erythroid cells stored in HTS or T1-1 for 32 or 45 days were also analyzed. As shown in FIG. 3B, enucleated erythroid cells stored in T1-1 maintained significantly better osmoscan properties than enucleated erythroid cells stored in HTS. Specifically, enucleated erythroid cells stored in T1-1 for 45 days showed better osmoscan properties as compared to enucleated erythroid cells stored in HTS for 32 days.

These results unexpectedly demonstrate that the compositions provided herein are advantageous in maintaining cell integrity, preventing hemolysis, and providing for improved deformability when enucleated erythroid cells are stored for an extended period of time.

### Example 3: Stability Analysis of Cell Storage Formulations

### Materials and Methods

### Hemolysis Assay

The same hemolysis assay as described in Example 2 was used in these experiments.

### Cell Concentration Assay

Cell concentration was determined using flow cytometry (BD FACSLyric™ system; BD BIOSCIENCES) using BD Trucount™ tubes (BD BIOSCIENCES) and calculated based on the number of cells detected, pre-defined beads in the BD Trucount™ tube, and dilution factor, using the equation (cell concentration per mL=(number of cellular events/number of beads)×(lot specific bead count/sample volume (mL))× dilution factor.

### Tested Compositions

Six different batches of exemplary engineered enucleated erythroid cells comprising a first exogenous protein comprising 4-1BBL and a second exogenous protein comprising IL-15 linked to an extracellular portion of IL-15Rα on their surface in T1-1 and T1-1 supplemented with 0.2% w/v human serum albumin (HSA) were used in these experiments. T1-1 supplemented with 0.2% w/v HSA include a pharmaceutically acceptable aqueous buffered solution comprising: 79.4 mM sodium ion, 41.7 mM potassium ion, 0.05 mM calcium ion, 5.0 mM magnesium ion, 10.0 mM chloride ion, 9.9 mM phosphate ion, 5.0 mM bicarbonate ion, 24.8 mM HEPES, 99.2 mM lactobionate, 39.7 mM mannitol, 2.0 mM adenosine, 1.0 mM adenine, and 0.20% w/v HSA, pH˜7.57.

### Results

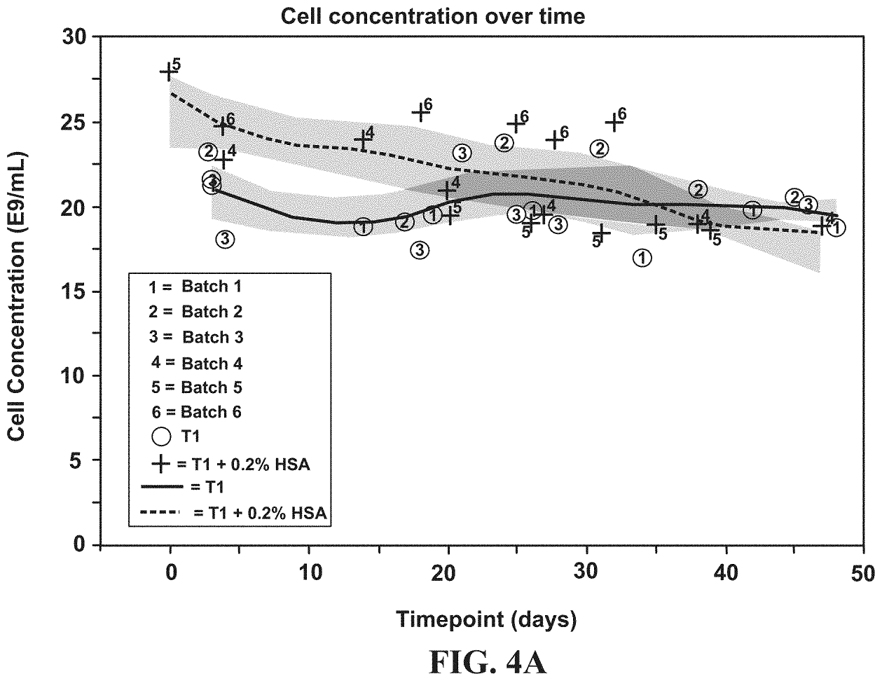

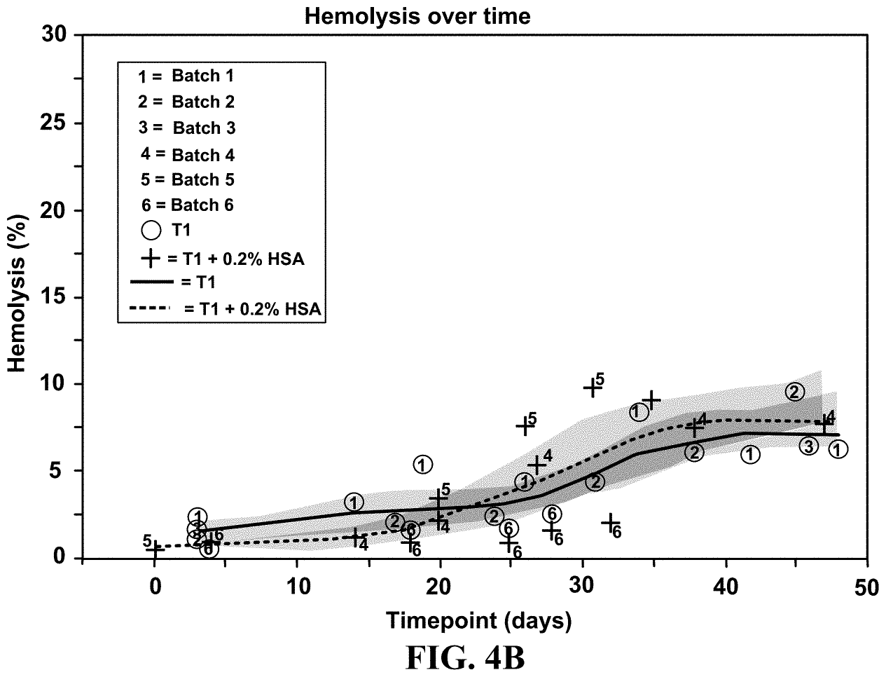

Engineered enucleated erythroid cells comprising a first exogenous protein comprising 4-1BBL and a second exogenous protein comprising IL-15 linked to an extracellular portion of IL-15Rα on their surface were used in these stability studies. The cells were produced in bioreactors of 50-liter scale, filtered, and processed into T1-1 or T1-1 supplemented with 0.2% HSA at a concentration of 1.5-3×10⁹ cells/mL and vialed into a glass vial closure system. The vials were stored at 2-8° C. and protected from light. At the indicated storage timepoints (days), a fresh vial was transferred to room temperature and submitted to stability testing of cell concentration by flow cytometry and hemolysis by spectrophotometry. The results (FIGS. 4A and 4B) show that the engineered enucleated erythroid cells were stable in T1-1, maintaining cell quantity and hemolysis within a safe level for 45+ days. The addition of HSA did not impact the stability results. These data suggest that T1-1 is a suitable formulation for stabilizing engineered enucleated erythroid cells comprising the exogenous proteins on their surface.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A composition comprising:
(a) a population of enucleated erythroid cells; and
(b) a pharmaceutically acceptable aqueous buffered solution having a pH of 6.5 to 8.5 and an osmolarity of 150 mOsm/L to 400 mOsm/L comprising:
about 5 mM to about 80 mM of a buffer;
about 5 mM to about 35 mM phosphate ion;
about 50 mM to about 160 mM sodium ion;
about 5 mM to about 60 mM potassium ion;
about 0.01 mM to about 10 mM calcium ion;
about 1 mM to about 20 mM magnesium ion; and
about 5 mM to about 60 mM of a non-ionic cell impermeant agent, wherein:
the pharmaceutically acceptable aqueous buffered solution comprises less than 5 mM glucose; and
optionally, the pharmaceutically acceptable aqueous buffered solution does not comprise one or more of: sucrose, a colloid, and an antioxidant.

2. (canceled)

3. The composition of claim 1, wherein the pharmaceutically acceptable aqueous buffered solution comprises about 20 mM to about 30 mM of the buffer.

4.-7. (canceled)

8. The composition of claim 1, wherein the pharmaceutically acceptable aqueous buffered solution comprises about 5 mM to about 15 mM phosphate ion.

9.-10. (canceled)

11. The composition of claim 1, wherein the pharmaceutically acceptable aqueous buffered solution comprises about 70 mM to about 120 mM sodium ion.

12.-13. (canceled)

14. The composition of claim 1, wherein the pharmaceutically acceptable aqueous buffered solution comprises about 30 mM to about 50 mM potassium ion.

15.-16. (canceled)

17. The composition of claim 1, wherein the pharmaceutically acceptable aqueous buffered solution comprises about 0.01 mM to about 0.5 mM calcium ion.

18.-19. (canceled)

20. The composition of claim 1, wherein the pharmaceutically acceptable aqueous buffered solution comprises about 3 mM to about 7 mM magnesium ion.

21. (canceled)

22. The composition of claim 1, wherein the pharmaceutically acceptable aqueous buffered solution further comprises about 20 mM to about 120 mM of an anionic cell impermeant agent.

23.-27. (canceled)

28. The composition of claim 1, wherein the pharmaceutically acceptable aqueous buffered solution comprises about 30 mM to about 50 mM of the non-ionic cell impermeant agent.

29.-31. (canceled)

32. The composition of claim 1, wherein the pharmaceutically acceptable aqueous buffered solution comprises about 5 mM to about 15 mM chloride ion.

33. The composition of claim 1, wherein the pharmaceutically acceptable aqueous buffered solution further comprises one or more of: about 0.01 mM to about 5 mM of a nucleobase, about 0.01 mM to about 5 mM of a nucleoside, and about 0.01 mM to about 5 mM of a nucleotide.

34.-35. (canceled)

36. The composition of claim 1, wherein the pharmaceutically acceptable aqueous buffered solution further comprises about 3 mM to about 7 mM bicarbonate ion.

37. (canceled)

38. The composition of claim 1, wherein the pharmaceutically acceptable aqueous buffered solution further comprises about 0.01 mM to about 5 mM pyruvate.

39. The composition of claim 1, wherein the pharmaceutically acceptable aqueous buffered solution further comprises a poloxamer.

40.-43. (canceled)

44. The composition of claim 1, wherein the pharmaceutically acceptable aqueous buffered solution further comprises human serum albumin.

45.-46. (canceled)

47. The composition of claim 1, wherein the pharmaceutically acceptable aqueous buffered solution has a pH of about 7.0 to about 8.0.

48. (canceled)

49. The composition of claim 1, wherein the pharmaceutically acceptable aqueous buffered solution has an osmolarity of about 250 mOsm/L to about 400 mOsm/L.

50. (canceled)

51. The composition of claim 1, wherein the pharmaceutically acceptable aqueous buffered solution comprises less than 0.01 mM glucose.

52.-53. (canceled)

54. The composition of claim 1, wherein the composition comprises about 1.0×10⁹ to about 7.0×10⁹ enucleated erythroid cells/mL.

55.-58. (canceled)

59. The composition of claim 1, wherein the enucleated erythroid cells are engineered human enucleated erythroid cells.

60. The composition of claim 59, wherein the engineered human enucleated erythroid cells comprise one or more exogenous protein(s).

61. The composition of claim 60, wherein the engineered human enucleated erythroid cells are click-conjugated human enucleated erythroid cells.

62. The composition of claim 60, wherein the engineered human enucleated erythroid cells have been hypotonically loaded.

63. The composition of claim 60, wherein the engineered human enucleated erythroid cells have been loaded by physical manipulation.

64. The composition of claim 60, wherein one of the one or more exogenous protein(s) is present in the cytosol of the engineered human enucleated erythroid cells.

65. The composition of claim 60, wherein one of the one or more exogenous protein is a protein present on the membrane of the engineered human enucleated erythroid cells.

66. (canceled)

67. The composition of claim 1, wherein storage of the composition at about 2° C. to about 10° C. for 30 days to about 100 days results in less than 10% hemolysis.

68. (canceled)

69. The composition of claim 1, wherein storage of the composition at about 2° C. to about 10° C. for 30 days to about 100 days results in less than a 10% decrease in cell density.

70.-74. (canceled)

75. A method of treating a subject, the method comprising:
(i) providing a composition of claim 1 that has been stored at a temperature of about 2° C. to about 10° C. for a period of time; and
(ii) administering the composition of step (i) to a subject in need thereof.

76.-88. (canceled)

89. A method of treating a subject, the method comprising administering the composition of claim 1 to a subject in need thereof.

90.-102. (canceled)

103. A method of making a composition, the method comprising:
(i) providing a population of enucleated erythroid cells; and
(ii) resuspending the population of enucleated erythroid cells in a pharmaceutically acceptable aqueous buffered solution having a pH of 6.5 to 8.5 and an osmolarity of 150 mOsm/L to 400 mOsm/L comprising:
about 5 mM to about 80 mM of a buffer;
about 5 mM to about 35 mM phosphate ion;
about 50 mM to about 160 mM sodium ion;
about 5 mM to about 60 mM potassium ion;
about 0.01 mM to about 10 mM calcium ion;
about 1 mM to about 20 mM magnesium ion; and
about 5 mM to about 60 mM of a non-ionic cell impermeant agent, wherein:
the pharmaceutically acceptable aqueous buffered solution comprises less than 5 mM glucose; and
optionally, the pharmaceutically acceptable aqueous buffered solution does not comprise one or more of: sucrose, a colloid, and an antioxidant.

104.-178. (canceled)
